(19) United States
(12) Patent Application Publication
(10) Pub. No.: US 2013/0121962 A1
(43) Pub. Date: May 16, 2013

(54) METHODS OF TREATING UROGENITAL-NEUROLOGICAL DISORDERS USING INTERLEUKIN RETARGETED ENDOPEPIDASES

(71) Applicant: ALLERGAN, INC., IRVINE, CA (US)

(72) Inventors: Joseph Francis, Aliso Viejo, CA (US); Dean G. Stathakis, Irvine, CA (US)

(73) Assignee: ALLERGAN, INC., Irvine, CA (US)

(21) Appl. No.: 13/721,621

(22) Filed: Dec. 20, 2012

Related U.S. Application Data
(63) Continuation of application No. 12/781,106, filed on May 17, 2010, now abandoned.
(60) Provisional application No. 61/182,216, filed on May 29, 2009.

(51) Int. Cl. C07K 14/54; C12N 9/52; C07K 14/33; C07K 14/475; C07K 14/485

(52) U.S. Cl. CPC C07K 14/54; C07K 14/475; C07K 14/485; C07K 14/33; C12N 9/52
USPC 424/85.2; 424/197.11

(57) ABSTRACT

The present specification discloses TVEMPs, compositions comprising such toxins and methods of treating urogenital-neurological disorders in a mammal using such TVEMPs and compositions.

### CROSS REFERENCES TO RELATED APPLICATIONS

This application is a Continuation of U.S. patent application Ser. No. 12/781,106, filed on May 17, 2010, which claims the benefit of U.S. Provisional Patent Application Ser. No. 61/182,216, filed May 29, 2009, both of which are hereby incorporated by reference in their entirety.

The ability of Clostridial toxins, such as, e.g., Botulinum neurotoxins (BoNTs), Botulinum neurotoxin serotype A (BoNT/A), Botulinum neurotoxin serotype B (BoNT/B), Botulinum neurotoxin serotype C1 (BoNT/C1), Botulinum neurotoxin serotype D (BoNT/D), Botulinum neurotoxin serotype E (BoNT/E), Botulinum neurotoxin serotype F (BoNT/F), and Botulinum neurotoxin serotype G (BoNT/G), and Tetanus neurotoxin (TeNT), to inhibit neuronal transmission are being exploited in a wide variety of therapeutic and cosmetic applications, see e.g., William J. Lipham, COSMETIC AND CLINICAL APPLICATIONS OF BOTULINUM TOXIN (Slack, Inc., 2004). Clostridial toxins commercially available as pharmaceutical compositions include, BoNT/A preparations, such as, e.g., BOTOX® (Allergan, Inc., Irvine, Calif.), DYSPORT®/RELOXIN®, (Beaufour Ipsen, Porton Down, England), NEURONOX® (Medy-Tox, Inc., Ochang-myeon, South Korea) BTX-A (Lanzhou Institute Biological Products, China) and XEOMIN® (Merz Pharmaceuticals, GmbH., Frankfurt, Germany); and BoNT/B preparations, such as, e.g., MYOBLOC™/NEUROBLOC™ (Elan Pharmaceuticals, San Francisco, Calif.). As an example, BOTOX® is currently approved in one or more countries for the following indications: achalasia, adult spasticity, anal fissure, back pain, blepharospasm, bruxism, cervical dystonia, essential tremor, glabellar lines or hyperkinetic facial lines, headache, hemifacial spasm, hyperactivity of bladder, hyperhidrosis, juvenile cerebral palsy, multiple sclerosis, myoclonic disorders, nasal labial lines, spasmodic dysphonia, strabismus and VII nerve disorder.

Clostridial toxin therapies are successfully used for many indications. Generally, administration of a Clostridial toxin treatment is well tolerated. However, toxin administration in some applications can be challenging because of the larger doses required to achieve a beneficial effect. Larger doses can increase the likelihood that the toxin may move through the interstitial fluids and the circulatory systems, such as, e.g., the cardiovascular system and the lymphatic system, of the body, resulting in the undesirable dispersal of the toxin to areas not targeted for toxin treatment. Such dispersal can lead to undesirable side effects, such as, e.g., inhibition of neurotransmitter release in neurons not targeted for treatment or paralysis of a muscle not targeted for treatment. For example, a patient administered a therapeutically effective amount of a BoNT/A treatment into the neck muscles for torticollis may develop dysphagia because of dispersal of the toxin into the oropharynx. As another example, a patient administered a therapeutically effective amount of a BoNT/A treatment into the bladder for overactive bladder may develop dry mouth and/or dry eyes. Thus, there remains a need for improved Clostridial toxins that are effective at the site of treatment, but have negligible to minimal effects in areas not targeted for a toxin treatment.

A Clostridial toxin treatment inhibits neurotransmitter release by disrupting the exocytotic process used to secret the neurotransmitter into the synaptic cleft. There is a great desire by the pharmaceutical industry to expand the use of Clostridial toxin therapies beyond its current myo-relaxant applications to treat other nerve-based ailments, such as, e.g., various kinds of chronic pain, neurogenic inflammation and urogentital disorders, as well as other disorders, such as, e.g., pancreatitis. One approach that is currently being exploited to expand Clostridial toxin-based therapies involves modifying a Clostridial toxin so that the modified toxin has an altered cell targeting capability for a non-Clostridial toxin target cell. This re-targeted capability is achieved by replacing a naturally-occurring targeting domain of a Clostridial toxin with a targeting domain showing a preferential binding activity for a non-Clostridial toxin receptor present in a non-Clostridial toxin target cell. Such modifications to a targeting domain result in a Clostridial toxin chimeric called a Targeted Vesicular Exocytosis Modulating Protein (TVEMP) that is able to selectively bind to a non-Clostridial toxin receptor (target receptor) present on a non-Clostridial toxin target cell (re-targeted). A Clostridial toxin chimeric with a targeting activity for a non-Clostridial toxin target cell can bind to a receptor present on the non-Clostridial toxin target cell, translocate into the cytoplasm, and exert its proteolytic effect on the SNARE complex of the non-Clostridial toxin target cell.

The present specification discloses TVEMP compositions and methods for treating an individual suffering from a neuron-mediated urogenital disorder. This is accomplished by administering a therapeutically effective amount of a composition comprising a TVEMP to an individual in need thereof. The disclosed methods provide a safe, inexpensive, out patient-based treatment for the treatment of urogenital-neurological disorders.

Thus, aspects of the present invention provide a composition comprising a TVEMP comprising a retargeted peptide binding domain, a Clostridial toxin translocation domain and a Clostridial toxin enzymatic domain. A composition comprising a TVEMP can be a pharmaceutical composition. Such a pharmaceutical composition can comprise, in addition to a TVEMP, a pharmaceutical carrier, a pharmaceutical component, or both.

Other aspects of the present invention provide a method of treating urogenital-neurological disorder in a mammal, the method comprising the step of administering to the mammal a therapeutically effective amount of a composition including a TVEMP comprising a retargeted peptide binding domain, a Clostridial toxin translocation domain and a Clostridial toxin enzymatic domain.

Other aspects of the present invention provide a manufacturing of a medicament for treating urogenital-neurological disorder in a mammal in need thereof, the medicament comprising a TVEMP including a retargeted peptide binding domain, a Clostridial toxin translocation domain and a Clostridial toxin enzymatic domain.

Other aspects of the present invention provide a use of a composition for treating urogenital-neurological disorder in a mammal in need thereof, the use comprising the step of administering to the mammal in need thereof a therapeutically effective amount of a composition, wherein the composition comprises a TVEMP including a retargeted peptide binding domain, a Clostridial toxin translocation domain and a Clostridial toxin enzymatic domain and wherein administration of the composition reduces a symptom of the urogenital-neurological disorder, thereby treating the mammal.

### BRIEF DESCRIPTION OF THE DRAWINGS

FIG. 1 shows a schematic of the current paradigm of neurotransmitter release and Clostridial toxin intoxication in a central and peripheral neuron. FIG. 1A shows a schematic for the neurotransmitter release mechanism of a central and peripheral neuron. The release process can be described as comprising two steps: 1) vesicle docking, where the vesicle-bound SNARE protein of a vesicle containing neurotransmitter molecules associates with the membrane-bound SNARE proteins located at the plasma membrane; and 2) neurotransmitter release, where the vesicle fuses with the plasma membrane and the neurotransmitter molecules are exocytosed. FIG. 1B shows a schematic of the intoxication mechanism for tetanus and botulinum toxin activity in a central and peripheral neuron. This intoxication process can be described as comprising four steps: 1) receptor binding, where a Clostridial toxin binds to a Clostridial receptor system and initiates the intoxication process; 2) complex internalization, where after toxin binding, a vesicle containing the toxin/receptor system complex is endocytosed into the cell; 3) light chain translocation, where multiple events are thought to occur, including, e.g., changes in the internal pH of the vesicle, formation of a channel pore comprising the H_{N} domain of the Clostridial toxin heavy chain, separation of the Clostridial toxin light chain from the heavy chain, and release of the active light chain and 4) enzymatic target modification, where the activate light chain of Clostridial toxin proteolytically cleaves its target SNARE substrate, such as, e.g., SNAP-25, VAMP or Syntaxin, thereby preventing vesicle docking and neurotransmitter release.

FIG. 2 shows the domain organization of naturally-occurring Clostridial toxins. The single-chain form depicts the amino to carboxyl linear organization comprising an enzymatic domain, a translocation domain, and a retargeted peptide binding domain. The di-chain loop region located between the translocation and enzymatic domains is depicted by the double SS bracket. This region comprises an endogenous di-chain loop protease cleavage site that upon proteolytic cleavage with a naturally-occurring protease, such as, e.g., an endogenous Clostridial toxin protease or a naturally-occurring protease produced in the environment, converts the single-chain form of the toxin into the di-chain form. Above the single-chain form, the HCC region of the Clostridial toxin binding domain is depicted. This region comprises the β-trefoil domain which comprises in an amino to carboxyl linear organization an α-fold, a β4/β5 hairpin turn, a β-fold, a β8/β9 hairpin turn and a γ-fold.

FIG. 3 shows TVEMPs with an enhanced targeting domain located at the amino terminus of the modified toxin. FIG. 3A depicts the single-chain polypeptide form of a TVEMP with an amino to carboxyl linear organization comprising a binding element, a translocation element, a di-chain loop region comprising an exogenous protease cleavage site (P), and a therapeutic element. Upon proteolytic cleavage with a P protease, the single-chain form of the toxin is converted to the di-chain form. FIG. 3B depicts the single polypeptide form of a TVEMP with an amino to carboxyl linear organization comprising a binding element, a therapeutic element, a di-chain loop region comprising an exogenous protease cleavage site (P), and a translocation element. Upon proteolytic cleavage with a P protease, the single-chain form of the toxin is converted to the di-chain form.

FIG. 4 shows TVEMPs with an enhanced targeting domain located between the other two domains. FIG. 4A depicts the single polypeptide form of a TVEMP with an amino to carboxyl linear organization comprising a therapeutic element, a di-chain loop region comprising an exogenous protease cleavage site (P), a binding element, and a translocation element. Upon proteolytic cleavage with a P protease, the single-chain form of the toxin is converted to the di-chain form. FIG. 4B depicts the single polypeptide form of a TVEMP with an amino to carboxyl linear organization comprising a translocation element, a di-chain loop region comprising an exogenous protease cleavage site (P), a binding element, and a therapeutic element. Upon proteolytic cleavage with a P protease, the single-chain form of the toxin is converted to the di-chain form. FIG. 4C depicts the single polypeptide form of a TVEMP with an amino to carboxyl linear organization comprising a therapeutic element, a binding element, a di-chain loop region comprising an exogenous protease cleavage site (P), and a translocation element. Upon proteolytic cleavage with a P protease, the single-chain form of the toxin is converted to the di-chain form. FIG. 4D depicts the single polypeptide form of a TVEMP with an amino to carboxyl linear organization comprising a translocation element, a binding element, a di-chain loop region comprising an exogenous protease cleavage site (P), and a therapeutic element. Upon proteolytic cleavage with a P protease, the single-chain form of the toxin is converted to the di-chain form.

FIG. 5 shows TVEMPs with an enhanced targeting domain located at the carboxyl terminus of the modified toxin. FIG. 5A depicts the single polypeptide form of a TVEMP with an amino to carboxyl linear organization comprising a therapeutic element, a di-chain loop region comprising an exogenous protease cleavage site (P), a translocation element, and a binding element. Upon proteolytic cleavage with a P protease, the single-chain form of the toxin is converted to the di-chain form. FIG. 5B depicts the single polypeptide form of a TVEMP with an amino to carboxyl linear organization comprising a translocation element, a di-chain loop region comprising an exogenous protease cleavage site (P), a therapeutic element, and a binding element. Upon proteolytic cleavage with a P protease, the single-chain form of the toxin is converted to the di-chain form.

### DETAILED DESCRIPTION

Aspects of the present invention provide, in part, a TVEMP. As used herein, a “Targeted Vesicular Exocytosis Modulating Protein” is synonomous with “TVEMP” and refers to any molecule comprising a retargeted peptide binding domain, a Clostridial toxin translocation domain and a Clostridial toxin enzymatic domain. Exemplary TVEMPs useful to practice aspects of the present invention are disclosed in, e.g., Steward, L. E. et al., Modified Clostridial Toxins with Enhanced Translocation Capabilities and Altered Targeting Activity For Non-Clostridial Toxin Target Cells, U.S. patent application Ser. No. 11/776,075 (Jul. 11, 2007); Dolly, J. O. et al., Activatable Clostridial Toxins, U.S. patent application Ser. No. 11/829,475 (Jul. 27, 2007); Foster, K. A. et al., Fusion Proteins, International Patent Publication WO 2006/059093 (Jun. 8, 2006); and Foster, K. A. et al., Non-Cytotoxic Protein Conjugates, International Patent Publication WO 2006/059105 (Jun. 8, 2006), each of which is incorporated by reference in its entirety.

Clostridia toxins produced by Clostridium botulinum, Clostridium tetani, Clostridium baratii and Clostridium butyricum are the most widely used in therapeutic and cosmetic treatments of humans and other mammals. Strains of C. botulinum produce seven antigenically-distinct types of Botulinum toxins (BoNTs), which have been identified by investigating botulism outbreaks in man (BoNT/A, /B, /E and /F), animals (BoNT/C1 and /D), or isolated from soil (BoNT/G). BoNTs possess approximately 35% amino acid identity with each other and share the same functional domain organization and overall structural architecture. It is recognized by those of skill in the art that within each type of Clostridial toxin there can be subtypes that differ somewhat in their amino acid sequence, and also in the nucleic acids encoding these proteins. For example, there are presently four BoNT/A subtypes, BoNT/A1, BoNT/A2, BoNT/A3 and BoNT/A4, with specific subtypes showing approximately 89% amino acid identity when compared to another BoNT/A subtype. While all seven BoNT serotypes have similar structure and pharmacological properties, each also displays heterogeneous bacteriological characteristics. In contrast, tetanus toxin (TeNT) is produced by a uniform group of C. tetani. Two other species of Clostridia, C. baratii and C. butyricum, also produce toxins, BaNT and BuNT respectively, which are similar to BoNT/F and BoNT/E, respectively.

Each mature di-chain molecule comprises three functionally distinct domains: 1) an enzymatic domain located in the light chain (LC) that includes a metalloprotease region containing a zinc-dependent endopeptidase activity which specifically targets core components of the neurotransmitter release apparatus; 2) a translocation domain (H_{N}) contained within the amino-terminal half of the heavy chain (HC) that facilitates release of the LC from intracellular vesicles into the cytoplasm of the target cell; and 3) a binding domain (H_{C}) found within the carboxyl-terminal half of the HC that determines the binding activity and binding specificity of the toxin to the receptor complex located at the surface of the target cell. The H_{C} domain comprises two distinct structural features of roughly equal size that indicate function and are designated the H_{CN} and H_{CC} subdomains. Table 1 gives approximate boundary regions for each domain found in exemplary Clostridial toxins.

TABLE 1
Clostridial Toxin Reference Sequences and Regions

| Toxin | SEQ ID NO: | LC | H_{N} | H_{C} |
|---|---|---|---|---|
| BoNT/A | 1 | M1-K448 | A449-K871 | N872-L1296 |
| BoNT/B | 2 | M1-K441 | A442-S858 | E859-E1291 |
| BoNT/C1 | 3 | M1-K449 | T450-N866 | N867-E1291 |
| BoNT/D | 4 | M1-R445 | D446-N862 | S863-E1276 |
| BoNT/E | 5 | M1-R422 | K423-K845 | R846-K1252 |
| BoNT/F | 6 | M1-K439 | A440-K864 | K865-E1274 |
| BoNT/G | 7 | M1-K446 | S447-S863 | N864-E1297 |
| TeNT | 8 | M1-A457 | S458-V879 | I880-D1315 |
| BaNT | 9 | M1-K431 | N432-I857 | I858-E1268 |
| BuNT | 10 | M1-R422 | K423-I847 | Y1086-K1251 |

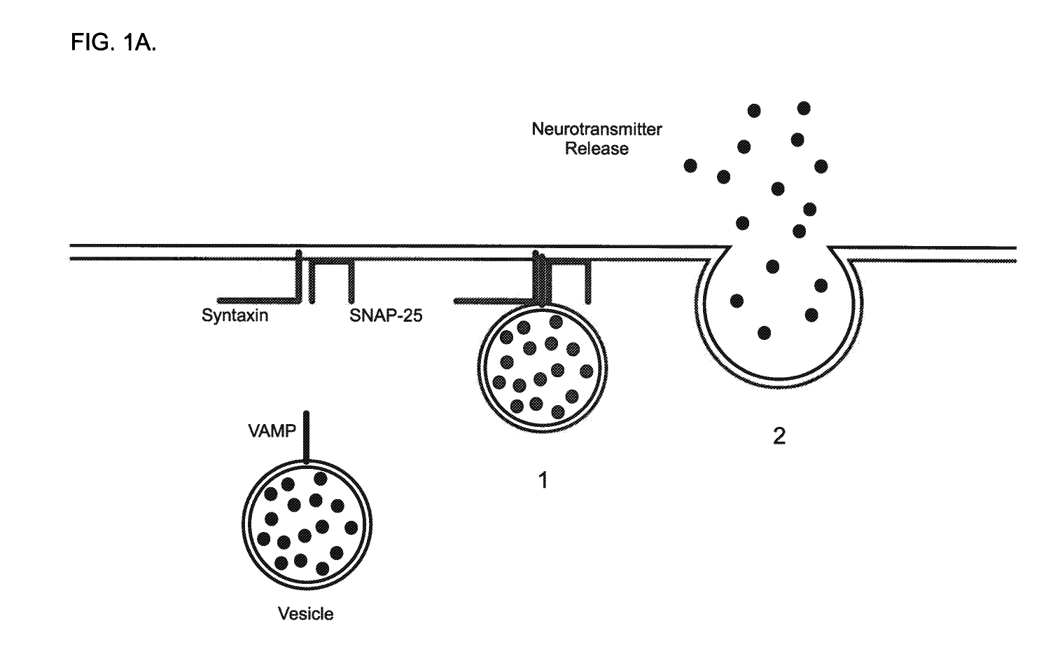

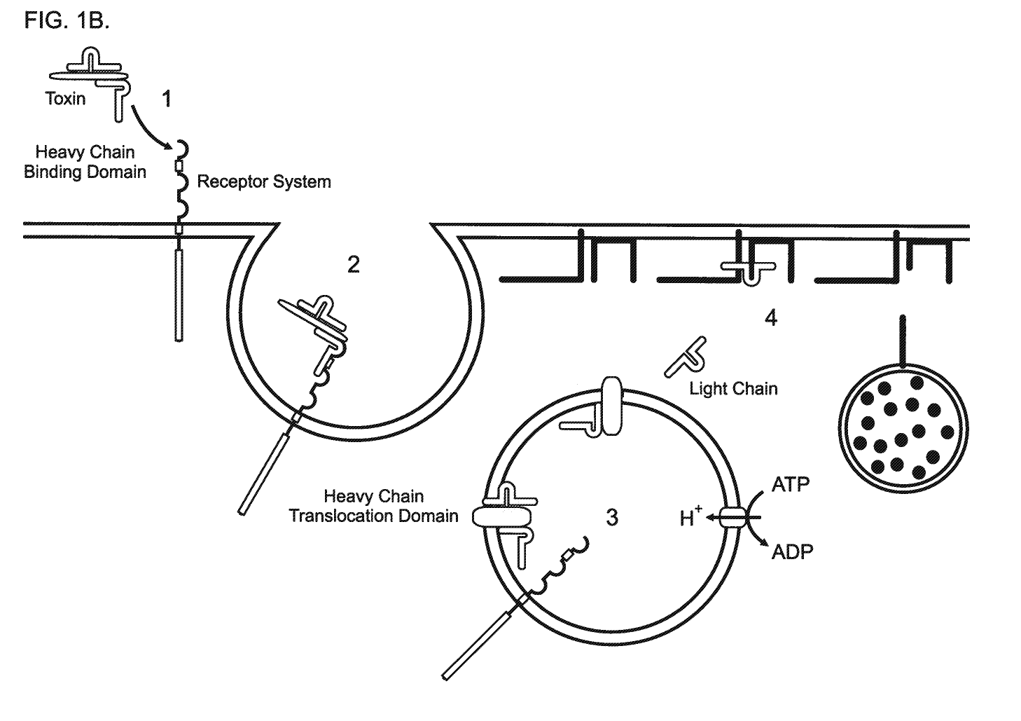

The binding, translocation and enzymatic activity of these three functional domains are all necessary for toxicity. While all details of this process are not yet precisely known, the overall cellular intoxication mechanism whereby Clostridial toxins enter a neuron and inhibit neurotransmitter release is similar, regardless of serotype or subtype. Although the applicants have no wish to be limited by the following description, the intoxication mechanism can be described as comprising at least four steps: 1) receptor binding, 2) complex internalization, 3) light chain translocation, and 4) enzymatic target modification (see FIG. 1). The process is initiated when the H_{C} domain of a Clostridial toxin binds to a toxin-specific receptor system located on the plasma membrane surface of a target cell. The binding specificity of a receptor complex is thought to be achieved, in part, by specific combinations of gangliosides and protein receptors that appear to distinctly comprise each Clostridial toxin receptor complex. Once bound, the toxin/receptor complexes are internalized by endocytosis and the internalized vesicles are sorted to specific intracellular routes. The translocation step appears to be triggered by the acidification of the vesicle compartment. This process seems to initiate two important pH-dependent structural rearrangements that increase hydrophobicity and promote formation di-chain form of the toxin. Once activated, light chain endopeptidase of the toxin is released from the intracellular vesicle into the cytosol where it appears to specifically target one of three known core components of the neurotransmitter release apparatus. These core proteins, vesicle-associated membrane protein (VAMP)/synaptobrevin, synaptosomal-associated protein of 25 kDa (SNAP-25) and Syntaxin, are necessary for synaptic vesicle docking and fusion at the nerve terminal and constitute members of the soluble N-ethylmaleimide-sensitive factor-attachment protein-receptor (SNARE) family. BoNT/A and BoNT/E cleave SNAP-25 in the carboxyl-terminal region, releasing a nine or twenty-six amino acid segment, respectively, and BoNT/C1 also cleaves SNAP-25 near the carboxyl-terminus. The botulinum serotypes BoNT/B, BoNT/D, BoNT/F and BoNT/G, and tetanus toxin, act on the conserved central portion of VAMP, and release the amino-terminal portion of VAMP into the cytosol. BoNT/C1 cleaves syntaxin at a single site near the cytosolic membrane surface. The selective proteolysis of synaptic SNAREs accounts for the block of neurotransmitter release caused by Clostridial toxins in vivo. The SNARE protein targets of Clostridial toxins are common to exocytosis in a variety of non-neuronal types; in these cells, as in neurons, light chain peptidase activity inhibits exocytosis, see, e.g., Yann Humeau et al., How Botulinum and Tetanus Neurotoxins Block Neurotransmitter Release, 82(5) Biochimie. 427-446 (2000); Kathryn Turton et al., Botulinum and Tetanus Neurotoxins: Structure, Function and Therapeutic Utility, 27(11) Trends Biochem. Sci. 552-558. (2002); Giovanna Lalli et al., The Journey of Tetanus and Botulinum Neurotoxins in Neurons, 11(9) Trends Microbiol. 431-437, (2003).

In an aspect of the invention, a TVEMP comprises, in part, a Clostridial toxin enzymatic domain. As used herein, the term “Clostridial toxin enzymatic domain” refers to any Clostridial toxin polypeptide that can execute the enzymatic target modification step of the intoxication process. Thus, a Clostridial toxin enzymatic domain specifically targets a Clostridial toxin substrate and encompasses the proteolytic cleavage of a Clostridial toxin substrate, such as, e.g., SNARE proteins like a SNAP-25 substrate, a VAMP substrate and a Syntaxin substrate. Non-limiting examples of a Clostridial toxin enzymatic domain include, e.g., a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, and a BuNT enzymatic domain. Other non-limiting examples of a Clostridial toxin enzymatic domain include, e.g., amino acids 1-448 of SEQ ID NO: 1, amino acids 1-441 of SEQ ID NO: 2, amino acids 1-449 of SEQ ID NO: 3, amino acids 1-445 of SEQ ID NO: 4, amino acids 1-422 of SEQ ID NO: 5, amino acids 1-439 of SEQ ID NO: 6, amino acids 1-446 of SEQ ID NO: 7, amino acids 1-457 of SEQ ID NO: 8, amino acids 1-431 of SEQ ID NO: 9, and amino acids 1-422 of SEQ ID NO: 10.

A Clostridial toxin enzymatic domain includes, without limitation, naturally occurring Clostridial toxin enzymatic domain variants, such as, e.g., Clostridial toxin enzymatic domain isoforms and Clostridial toxin enzymatic domain subtypes; and non-naturally occurring Clostridial toxin enzymatic domain variants, such as, e.g., conservative Clostridial toxin enzymatic domain variants, non-conservative Clostridial toxin enzymatic domain variants, Clostridial toxin enzymatic domain chimerics, active Clostridial toxin enzymatic domain fragments thereof, or any combination thereof.

As used herein, the term “Clostridial toxin enzymatic domain variant,” whether naturally-occurring or non-naturally-occurring, refers to a Clostridial toxin enzymatic domain that has at least one amino acid change from the corresponding region of the disclosed reference sequences (Table 1) and can be described in percent identity to the corresponding region of that reference sequence. Unless expressly indicated, Clostridial toxin enzymatic domain variants useful to practice disclosed embodiments are variants that execute the enzymatic target modification step of the intoxication process. As non-limiting examples, a BoNT/A enzymatic domain variant comprising amino acids 1-448 of SEQ ID NO: 1 will have at least one amino acid difference, such as, e.g., an amino acid substitution, deletion or addition, as compared to the amino acid region 1-448 of SEQ ID NO: 1; a BoNT/B enzymatic domain variant comprising amino acids 1-441 of SEQ ID NO: 2 will have at least one amino acid difference, such as, e.g., an amino acid substitution, deletion or addition, as compared to the amino acid region 1-441 of SEQ ID NO: 2; a BoNT/C1 enzymatic domain variant comprising amino acids 1-449 of SEQ ID NO: 3 will have at least one amino acid difference, such as, e.g., an amino acid substitution, deletion or addition, as compared to the amino acid region 1-449 of SEQ ID NO: 3; a BoNT/D enzymatic domain variant comprising amino acids 1-445 of SEQ ID NO: 4 will have at least one amino acid difference, such as, e.g., an amino acid substitution, deletion or addition, as compared to the amino acid region 1-445 of SEQ ID NO: 4; a BoNT/E enzymatic domain variant comprising amino acids 1-422 of SEQ ID NO: 5 will have at least one amino acid difference, such as, e.g., an amino acid substitution, deletion or addition, as compared to the amino acid region 1-422 of SEQ ID NO: 5; a BoNT/F enzymatic domain variant comprising amino acids 1-439 of SEQ ID NO: 6 will have at least one amino acid difference, such as, e.g., an amino acid substitution, deletion or addition, as compared to the amino acid region 1-439 of SEQ ID NO: 6; a BoNT/G enzymatic domain variant comprising amino acids 1-446 of SEQ ID NO: 7 will have at least one amino acid difference, such as, e.g., an amino acid substitution, deletion or addition, as compared to the amino acid region 1-446 of SEQ ID NO: 7; and a TeNT enzymatic domain variant comprising amino acids 1-457 of SEQ ID NO: 8 will have at least one amino acid difference, such as, e.g., an amino acid substitution, deletion or addition, as compared to the amino acid region 1-457 of SEQ ID NO: 8.

It is recognized by those of skill in the art that within each serotype of Clostridial toxin there can be naturally occurring Clostridial toxin enzymatic domain variants that differ somewhat in their amino acid sequence, and also in the nucleic acids encoding these proteins. For example, there are presently five BoNT/A subtypes, BoNT/A1, BoNT/A2, BoNT/A3, BoNT/A4, and BoNT/A5, with specific enzymatic domain subtypes showing approximately 95% amino acid identity when compared to another BoNT/A enzymatic domain subtype. As used herein, the term “naturally occurring Clostridial toxin enzymatic domain variant” refers to any Clostridial toxin enzymatic domain produced by a naturally-occurring process, including, without limitation, Clostridial toxin enzymatic domain isoforms produced from alternatively-spliced transcripts, Clostridial toxin enzymatic domain isoforms produced by spontaneous mutation and Clostridial toxin enzymatic domain subtypes. A naturally occurring Clostridial toxin enzymatic domain variant can function in substantially the same manner as the reference Clostridial toxin enzymatic domain on which the naturally occurring Clostridial toxin enzymatic domain variant is based, and can be substituted for the reference Clostridial toxin enzymatic domain in any aspect of the present invention.

A non-limiting example of a naturally occurring Clostridial toxin enzymatic domain variant is a Clostridial toxin enzymatic domain isoform such as, e.g., a BoNT/A enzymatic domain isoform, a BoNT/B enzymatic domain isoform, a BoNT/C1 enzymatic domain isoform, a BoNT/D enzymatic domain isoform, a BoNT/E enzymatic domain isoform, a BoNT/F enzymatic domain isoform, a BoNT/G enzymatic domain isoform, and a TeNT enzymatic domain isoform. Another non-limiting example of a naturally occurring Clostridial toxin enzymatic domain variant is a Clostridial toxin enzymatic domain subtype such as, e.g., an enzymatic domain from subtype BoNT/A1, BoNT/A2, BoNT/A3, BoNT/A4 and BoNT/A5; an enzymatic domain from subtype BoNT/B1, BoNT/B2, BoNT/B bivalent and BoNT/B nonproteolytic; an enzymatic domain from subtype BoNT/C1-1 and BoNT/C1-2; an enzymatic domain from subtype BoNT/E1, BoNT/E2 and BoNT/E3; and an enzymatic domain from subtype BoNT/F1, BoNT/F2, BoNT/F3 and BoNT/F4.

As used herein, the term “non-naturally occurring Clostridial toxin enzymatic domain variant” refers to any Clostridial toxin enzymatic domain produced with the aid of human manipulation, including, without limitation, Clostridial toxin enzymatic domains produced by genetic engineering using random mutagenesis or rational design and Clostridial toxin enzymatic domains produced by chemical synthesis. Non-limiting examples of non-naturally occurring Clostridial toxin enzymatic domain variants include, e.g., conservative Clostridial toxin enzymatic domain variants, non-conservative Clostridial toxin enzymatic domain variants, Clostridial toxin enzymatic domain chimeric variants and active Clostridial toxin enzymatic domain fragments.

As used herein, the term “conservative Clostridial toxin enzymatic domain variant” refers to a Clostridial toxin enzymatic domain that has at least one amino acid substituted by another amino acid or an amino acid analog that has at least one property similar to that of the original amino acid from the reference Clostridial toxin enzymatic domain sequence (Table 1). Examples of properties include, without limitation, similar size, topography, charge, hydrophobicity, hydrophilicity, lipophilicity, covalent-bonding capacity, hydrogen-bonding capacity, a physicochemical property, of the like, or any combination thereof. A conservative Clostridial toxin enzymatic domain variant can function in substantially the same manner as the reference Clostridial toxin enzymatic domain on which the conservative Clostridial toxin enzymatic domain variant is based, and can be substituted for the reference Clostridial toxin enzymatic domain in any aspect of the present invention. Non-limiting examples of a conservative Clostridial toxin enzymatic domain variant include, e.g., conservative BoNT/A enzymatic domain variants, conservative BoNT/B enzymatic domain variants, conservative BoNT/C1 enzymatic domain variants, conservative BoNT/D enzymatic domain variants, conservative BoNT/E enzymatic domain variants, conservative BoNT/F enzymatic domain variants, conservative BoNT/G enzymatic domain variants, and conservative TeNT enzymatic domain variants.

As used herein, the term “non-conservative Clostridial toxin enzymatic domain variant” refers to a Clostridial toxin enzymatic domain in which 1) at least one amino acid is deleted from the reference Clostridial toxin enzymatic domain on which the non-conservative Clostridial toxin enzymatic domain variant is based; 2) at least one amino acid added to the reference Clostridial toxin enzymatic domain on which the non-conservative Clostridial toxin enzymatic domain is based; or 3) at least one amino acid is substituted by another amino acid or an amino acid analog that does not share any property similar to that of the original amino acid from the reference Clostridial toxin enzymatic domain sequence (Table 1). A non-conservative Clostridial toxin enzymatic domain variant can function in substantially the same manner as the reference Clostridial toxin enzymatic domain on which the non-conservative Clostridial toxin enzymatic domain variant is based, and can be substituted for the reference Clostridial toxin enzymatic domain in any aspect of the present invention. Non-limiting examples of a non-conservative Clostridial toxin enzymatic domain variant include, e.g., non-conservative BoNT/A enzymatic domain variants, non-conservative BoNT/B enzymatic domain variants, non-conservative BoNT/C1 enzymatic domain variants, non-conservative BoNT/D enzymatic domain variants, non-conservative BoNT/E enzymatic domain variants, non-conservative BoNT/F enzymatic domain variants, non-conservative BoNT/G enzymatic domain variants, and non-conservative TeNT enzymatic domain variants.

As used herein, the term “Clostridial toxin enzymatic domain chimeric” refers to a polypeptide comprising at least a portion of a Clostridial toxin enzymatic domain and at least a portion of at least one other polypeptide to form a toxin enzymatic domain with at least one property different from the reference Clostridial toxin enzymatic domains of Table 1, with the proviso that this Clostridial toxin enzymatic domain chimeric is still capable of specifically targeting the core components of the neurotransmitter release apparatus and thus participate in executing the overall cellular mechanism whereby a Clostridial toxin proteolytically cleaves a substrate. Such Clostridial toxin enzymatic domain chimerics are described in, e.g., Lance E. Steward et al., Leucine-based Motif and Clostridial Toxins, U.S. Patent Publication 2003/0027752 (Feb. 6, 2003); Lance E. Steward et al., Clostridial Neurotoxin Compositions and Modified Clostridial Neurotoxins, U.S. Patent Publication 2003/0219462 (Nov. 27, 2003); and Lance E. Steward et al., Clostridial Neurotoxin Compositions and Modified Clostridial Neurotoxins, U.S. Patent Publication 2004/0220386 (Nov. 4, 2004), each of which is incorporated by reference in its entirety.

As used herein, the term “active Clostridial toxin enzymatic domain fragment” refers to any of a variety of Clostridial toxin fragments comprising the enzymatic domain can be useful in aspects of the present invention with the proviso that these enzymatic domain fragments can specifically target the core components of the neurotransmitter release apparatus and thus participate in executing the overall cellular mechanism whereby a Clostridial toxin proteolytically cleaves a substrate. The enzymatic domains of Clostridial toxins are approximately 420-460 amino acids in length and comprise an enzymatic domain (Table 1). Research has shown that the entire length of a Clostridial toxin enzymatic domain is not necessary for the enzymatic activity of the enzymatic domain. As a non-limiting example, the first eight amino acids of the BoNT/A enzymatic domain (residues 1-8 of SEQ ID NO: 1) are not required for enzymatic activity. As another non-limiting example, the first eight amino acids of the TeNT enzymatic domain (residues 1-8 of SEQ ID NO: 8) are not required for enzymatic activity. Likewise, the carboxyl-terminus of the enzymatic domain is not necessary for activity. As a non-limiting example, the last 32 amino acids of the BoNT/A enzymatic domain (residues 417-448 of SEQ ID NO: 1) are not required for enzymatic activity. As another non-limiting example, the last 31 amino acids of the TeNT enzymatic domain (residues 427-457 of SEQ ID NO: 8) are not required for enzymatic activity. Thus, aspects of this embodiment can include Clostridial toxin enzymatic domains comprising an enzymatic domain having a length of, e.g., at least 350 amino acids, at least 375 amino acids, at least 400 amino acids, at least 425 amino acids or at least 450 amino acids. Other aspects of this embodiment can include Clostridial toxin enzymatic domains comprising an enzymatic domain having a length of, e.g., at most 350 amino acids, at most 375 amino acids, at most 400 amino acids, at most 425 amino acids or at most 450 amino acids.

Any of a variety of sequence alignment methods can be used to determine percent identity of naturally-occurring Clostridial toxin enzymatic domain variants and non-naturally-occurring Clostridial toxin enzymatic domain variants, including, without limitation, global methods, local methods and hybrid methods, such as, e.g., segment approach methods. Protocols to determine percent identity are routine procedures within the scope of one skilled in the art and from the teaching herein.

Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein Sequence Alignments by Iterative Refinement as Assessed by Reference to Structural Alignments, 264(4) J. Mol. Biol. 823-838 (1996).

Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics,:1428-1435 (2004).

Hybrid methods combine functional aspects of both global and local alignment methods. Non-limiting methods include, e.g., segment-to-segment comparison, see, e.g., Burkhard Morgenstern et al., Multiple DNA and Protein Sequence Alignment Based On Segment-To-Segment Comparison, 93(22) Proc. Natl. Acad. Sci. U.S.A. 12098-12103 (1996); T-Coffee, see, e.g., Cédric Notredame et al., T-Coffee: A Novel Algorithm for Multiple Sequence Alignment, 302(1) J. Mol. Biol. 205-217 (2000); MUSCLE, see, e.g., Robert C. Edgar, MUSCLE: Multiple Sequence Alignment With High Score Accuracy and High Throughput, 32(5) Nucleic Acids Res. 1792-1797 (2004); and DIALIGN-T, see, e.g., Amarendran R Subramanian et al., DIALIGN-T: An Improved Algorithm for Segment-Based Multiple Sequence Alignment, 6(1) BMC Bioinformatics 66 (2005).

The present specification describes various polypeptide variants where one amino acid is substituted for another, such as, e.g., Clostridial toxin variants, Clostridial toxin enzymatic domain variants, Clostridial toxin translocation domain variants, Clostridial toxin binding domain variants, non-Clostridial toxin binding domain variants, retargeted peptide binding domain variants, and protease cleavage site variants. A substitution can be assessed by a variety of factors, such as, e.g., the physic properties of the amino acid being substituted (Table 2) or how the original amino acid would tolerate a substitution (Table 3). The selections of which amino acid can be substituted for another amino acid in a polypeptide are known to a person of ordinary skill in the art.

TABLE 2
Amino Acid Properties

| Property | Amino Acids |
|---|---|
| Aliphatic | G, A, I, L, M, P, V |
| Aromatic | F, H, W, Y |
| C-beta branched | I, V, T |
| Hydrophobic | C, F, I, L, M, V, W |
| Small polar | D, N, P |
| Small non-polar | A, C, G, S, T |
| Large polar | E, H, K, Q, R, W, Y |
| Large non-polar | F, I, L, M, V |
| Charged | D, E, H, K, R |
| Uncharged | C, S, T |
| Negative | D, E |
| Positive | H, K, R |
| Acidic | D, E |
| Basic | K, R |
| Amide | N, Q |

TABLE 3
Amino Acid Substitutions

| Amino | Favored | | |
|---|---|---|---|
| Acid | Substitution | Neutral Substitutions | Disfavored substitution |
| A | G, S, T | C, E, I, K, M, L, P, Q, | D, F, H, N, Y, W |
| | | R, V | |
| C | F, S, Y, W | A, H, I, M, L, T, V | D, E, G, K, N, P, Q, R |
| D | E, N | G, H, K, P, Q, R, S, T | A, C, I, L, |
| E | D, K, Q | A, H, N, P, R, S, T | C, F, G, I, L, M, V, |
| | | | W, Y |
| F | M, L, W, Y | C, I, V | A, D, E, G, H, K, N, P, |
| | | | Q, R, S, T |
| G | A, S | D, K, N, P, Q, R | C, E, F, H, I, L, M, T, |
| | | | V, W, Y |
| H | N, Y | C, D, E, K, Q, R, S, | A, F, G, I, L, M, P, V |
| | | T, W | |
| I | V, L, M | A, C, T, F, Y | D, E, G, H, K, N, P, |
| | | | Q, R, S, W |
| K | Q, E, R | A, D, G, H, M, N, P, | C, F, I, L, |
| | | S, T | V, W, Y |
| L | F, I, M, V | A, C, W, Y | D, E, G, H, K, N, P, Q, |
| | | | R, S, T |
| M | F, I, L, V | A, C, R, Q, K, T, W, | D, E, G, H, |
| | | Y | N, P, S |
| N | D, H, S | E, G, K, Q, R, T | A, C, F, I, L, M, P, |
| | | | V, W, Y |
| P | — | A, D, E, G, K, Q, R, | C, F, H, I, L, M, N, |
| | | S, T | V, W, Y |
| Q | E, K, R | A, D, G, H, M, N, | C, F, I, L, V, W, Y |
| | | P, S, T | |
| R | K, Q | A, D, E, G, H, M, N, | C, F, I, L, V, W, Y |
| | | P, S, T | |
| S | A, N, T | C, D, E, G, H, K, P, | F, I, L, M, V, W, Y |
| | | Q, R, T | |
| T | S | A, C, D, E, H, I, K, | F, G, L, W, Y |
| | | M, N, P, Q, R, V | |
| V | I, L, M | A, C, F, T, Y | D, E, G, H, K, N, P, Q, |
| | | | R, S, W |
| W | F, Y | H, L, M | A, C, D, E, G, I, K, N, |
| | | | P, Q, R, S, T, V |
| Y | F, H, W | C, I, L, M, V | A, D, E, G, K, N, P, |
| | | | Q, R, S, T |

Matthew J. Betts and Robert, B. Russell, Amino Acid Properties and Consequences of Substitutions, pp. 289-316, In Bioinformatics for Geneticists, (eds Michael R. Barnes, Ian C. Gray, Wiley, 2003).

Thus, in an embodiment, a TVEMP disclosed in the present specification comprises a Clostridial toxin enzymatic domain. In an aspect of this embodiment, a Clostridial toxin enzymatic domain comprises a naturally occurring Clostridial toxin enzymatic domain variant, such as, e.g., a Clostridial toxin enzymatic domain isoform or a Clostridial toxin enzymatic domain subtype. In another aspect of this embodiment, a Clostridial toxin enzymatic domain comprises a non-naturally occurring Clostridial toxin enzymatic domain variant, such as, e.g., a conservative Clostridial toxin enzymatic domain variant, a non-conservative Clostridial toxin enzymatic domain variant, a Clostridial toxin chimeric enzymatic domain, an active Clostridial toxin enzymatic domain fragment, or any combination thereof.

In another embodiment, a Clostridial toxin enzymatic domain comprises a BoNT/A enzymatic domain. In an aspect of this embodiment, a BoNT/A enzymatic domain comprises amino acids 1-448 of SEQ ID NO: 1. In another aspect of this embodiment, a BoNT/A enzymatic domain comprises a naturally occurring BoNT/A enzymatic domain variant, such as, e.g., an enzymatic domain from a BoNT/A isoform or an enzymatic domain from a BoNT/A subtype. In another aspect of this embodiment, a BoNT/A enzymatic domain comprises amino acids 1-448 of a naturally occurring BoNT/A enzymatic domain variant of SEQ ID NO: 1, such as, e.g., amino acids 1-448 of a BoNT/A isoform of SEQ ID NO: 1 or amino acids 1-448 of a BoNT/A subtype of SEQ ID NO: 1. In still another aspect of this embodiment, a BoNT/A enzymatic domain comprises a non-naturally occurring BoNT/A enzymatic domain variant, such as, e.g., a conservative BoNT/A enzymatic domain variant, a non-conservative BoNT/A enzymatic domain variant, a BoNT/A chimeric enzymatic domain, an active BoNT/A enzymatic domain fragment, or any combination thereof. In still another aspect of this embodiment, a BoNT/A enzymatic domain comprises amino acids 1-448 of a non-naturally occurring BoNT/A enzymatic domain variant of SEQ ID NO: 1, such as, e.g., amino acids 1-448 of a conservative BoNT/A enzymatic domain variant of SEQ ID NO: 1, amino acids 1-448 of a non-conservative BoNT/A enzymatic domain variant of SEQ ID NO: 1, amino acids 1-448 of an active BoNT/A enzymatic domain fragment of SEQ ID NO: 1, or any combination thereof.

In other aspects of this embodiment, a BoNT/A enzymatic domain comprises a polypeptide having an amino acid identity of, e.g., at least 70%, at least 75%, at least 80%, at least 85%, at least 90%, at least 95%, or at least 97% to amino acids 1-448 of SEQ ID NO: 1; or at most 70%, at most 75%, at most 80%, at most 85%, at most 90%, at most 95%, or at most 97% to amino acids 1-448 of SEQ ID NO: 1. In yet other aspects of this embodiment, a BoNT/A enzymatic domain comprises a polypeptide having, e.g., at most 1, 2, 3, 4, 5, 6, 7, 8, 9, 10, 20, 30, 40, 50, or 100 non-contiguous amino acid deletions, additions, and/or substitutions relative to amino acids 1-448 of SEQ ID NO: 1; or at most 1, 2, 3, 4, 5, 6, 7, 8, 9, 10, 20, 30, 40, 50, or 100 non-contiguous amino acid deletions, additions, and/or substitutions relative to amino acids 1-448 of SEQ ID NO: 1. In still other aspects of this embodiment, a BoNT/A enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 1-448 of SEQ ID NO: 1; or at most 1, 2, 3, 4, 5, 6, 7, 8, 9, 10, 20, 30, 40, 50, or 100 contiguous amino acid deletions, additions, and/or substitutions relative to amino acids 1-448 of SEQ ID NO: 1.

In another embodiment, a Clostridial toxin enzymatic domain comprises a BoNT/B enzymatic domain. In an aspect of this embodiment, a BoNT/B enzymatic domain comprises amino acids 1-441 of SEQ ID NO: 2. In another aspect of this embodiment, a BoNT/B enzymatic domain comprises a naturally occurring BoNT/B enzymatic domain variant, such as, e.g., an enzymatic domain from a BoNT/B isoform or an enzymatic domain from a BoNT/B subtype. In another aspect of this embodiment, a BoNT/B enzymatic domain comprises amino acids 1-441 of a naturally occurring BoNT/B enzymatic domain variant of SEQ ID NO: 2, such as, e.g., amino acids 1-441 of a BoNT/B isoform of SEQ ID NO: 2 or amino acids 1-441 of a BoNT/B subtype of SEQ ID NO: 2. In still another aspect of this embodiment, a BoNT/B enzymatic domain comprises a non-naturally occurring BoNT/B enzymatic domain variant, such as, e.g., a conservative BoNT/B enzymatic domain variant, a non-conservative BoNT/B enzymatic domain variant, a BoNT/B chimeric enzymatic domain, an active BoNT/B enzymatic domain fragment, or any combination thereof. In still another aspect of this embodiment, a BoNT/B enzymatic domain comprises amino acids 1-441 of a non-naturally occurring BoNT/B enzymatic domain variant of SEQ ID NO: 2, such as, e.g., amino acids 1-441 of a conservative BoNT/B enzymatic domain variant of SEQ ID NO: 2, amino acids 1-441 of a non-conservative BoNT/B enzymatic domain variant of SEQ ID NO: 2, amino acids 1-441 of an active BoNT/B enzymatic domain fragment of SEQ ID NO: 2, or any combination thereof.

In other aspects of this embodiment, a BoNT/B enzymatic domain comprises a polypeptide having an amino acid identity of, e.g., at least 70%, at least 75%, at least 80%, at least 85%, at least 90%, at least 95%, or at least 97% to amino acids 1-441 of SEQ ID NO: 2; or at most 70%, at most 75%, at most 80%, at most 85%, at most 90%, at most 95%, or at most 97% to amino acids 1-441 of SEQ ID NO: 2. In yet other aspects of this embodiment, a BoNT/B enzymatic domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 1-441 of SEQ ID NO: 2; or at most 1, 2, 3, 4, 5, 6, 7, 8, 9, 10, 20, 30, 40, 50, or 100 non-contiguous amino acid deletions, additions, and/or substitutions relative to amino acids 1-441 of SEQ ID NO: 2. In still other aspects of this embodiment, a BoNT/B enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 1-441 of SEQ ID NO: 2; or at most 1, 2, 3, 4, 5, 6, 7, 8, 9, 10, 20, 30, 40, 50, or 100 contiguous amino acid deletions, additions, and/or substitutions relative to amino acids 1-441 of SEQ ID NO: 2.

In another embodiment, a Clostridial toxin enzymatic domain comprises a BoNT/C1 enzymatic domain. In an aspect of this embodiment, a BoNT/C1 enzymatic domain comprises amino acids 1-449 of SEQ ID NO: 3. In another aspect of this embodiment, a BoNT/C1 enzymatic domain comprises a naturally occurring BoNT/C1 enzymatic domain variant, such as, e.g., an enzymatic domain from a BoNT/C1 isoform or an enzymatic domain from a BoNT/C1 subtype. In another aspect of this embodiment, a BoNT/C1 enzymatic domain comprises amino acids 1-449 of a naturally occurring BoNT/C1 enzymatic domain variant of SEQ ID NO: 3, such as, e.g., amino acids 1-449 of a BoNT/C1 isoform of SEQ ID NO: 3 or amino acids 1-449 of a BoNT/C1 subtype of SEQ ID NO: 3. In still another aspect of this embodiment, a BoNT/C1 enzymatic domain comprises a non-naturally occurring BoNT/C1 enzymatic domain variant, such as, e.g., a conservative BoNT/C1 enzymatic domain variant, a non-conservative BoNT/C1 enzymatic domain variant, a BoNT/C1 chimeric enzymatic domain, an active BoNT/C1 enzymatic domain fragment, or any combination thereof. In still another aspect of this embodiment, a BoNT/C1 enzymatic domain comprises amino acids 1-449 of a non-naturally occurring BoNT/C1 enzymatic domain variant of SEQ ID NO: 3, such as, e.g., amino acids 1-449 of a conservative BoNT/C1 enzymatic domain variant of SEQ ID NO: 3, amino acids 1-449 of a non-conservative BoNT/C1 enzymatic domain variant of SEQ ID NO: 3, amino acids 1-449 of an active BoNT/C1 enzymatic domain fragment of SEQ ID NO: 3, or any combination thereof.

In other aspects of this embodiment, a BoNT/C1 enzymatic domain comprises a polypeptide having an amino acid identity of, e.g., at least 70%, at least 75%, at least 80%, at least 85%, at least 90%, at least 95%, or at least 97% to amino acids 1-449 of SEQ ID NO: 3; or at most 70%, at most 75%, at most 80%, at most 85%, at most 90%, at most 95%, or at most 97% to amino acids 1-449 of SEQ ID NO: 3. In yet other aspects of this embodiment, a BoNT/C1 enzymatic domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 1-449 of SEQ ID NO: 3; or at most 1, 2, 3, 4, 5, 6, 7, 8, 9, 10, 20, 30, 40, 50, or 100 non-contiguous amino acid deletions, additions, and/or substitutions relative to amino acids 1-449 of SEQ ID NO: 3. In other aspects of this embodiment, a BoNT/C1 enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 1-449 of SEQ ID NO: 3; or at most 1, 2, 3, 4, 5, 6, 7, 8, 9, 10, 20, 30, 40, 50, or 100 contiguous amino acid deletions, additions, and/or substitutions relative to amino acids 1-449 of SEQ ID NO: 3.

In another embodiment, a Clostridial toxin enzymatic domain comprises a BoNT/D enzymatic domain. In an aspect of this embodiment, a BoNT/D enzymatic domain comprises amino acids 1-445 of SEQ ID NO: 4. In another aspect of this embodiment, a BoNT/D enzymatic domain comprises a naturally occurring BoNT/D enzymatic domain variant, such as, e.g., an enzymatic domain from a BoNT/D isoform or an enzymatic domain from a BoNT/D subtype. In another aspect of this embodiment, a BoNT/D enzymatic domain comprises amino acids 1-445 of a naturally occurring BoNT/D enzymatic domain variant of SEQ ID NO: 4, such as, e.g., amino acids 1-445 of a BoNT/D isoform of SEQ ID NO: 4 or amino acids 1-445 of a BoNT/D subtype of SEQ ID NO: 4. In still another aspect of this embodiment, a BoNT/D enzymatic domain comprises a non-naturally occurring BoNT/D enzymatic domain variant, such as, e.g., a conservative BoNT/D enzymatic domain variant, a non-conservative BoNT/D enzymatic domain variant, a BoNT/D chimeric enzymatic domain, an active BoNT/D enzymatic domain fragment, or any combination thereof. In still another aspect of this embodiment, a BoNT/D enzymatic domain comprises amino acids 1-445 of a non-naturally occurring BoNT/D enzymatic domain variant of SEQ ID NO: 4, such as, e.g., amino acids 1-445 of a conservative BoNT/D enzymatic domain variant of SEQ ID NO: 4, amino acids 1-445 of a non-conservative BoNT/D enzymatic domain variant of SEQ ID NO: 4, amino acids 1-445 of an active BoNT/D enzymatic domain fragment of SEQ ID NO: 4, or any combination thereof.

In other aspects of this embodiment, a BoNT/D enzymatic domain comprises a polypeptide having an amino acid identity of, e.g., at least 70%, at least 75%, at least 80%, at least 85%, at least 90%, at least 95%, or at least 97% to amino acids 1-445 of SEQ ID NO: 4; or at most 70%, at most 75%, at most 80%, at most 85%, at most 90%, at most 95%, or at most 97% to amino acids 1-445 of SEQ ID NO: 4. In yet other aspects of this embodiment, a BoNT/D enzymatic domain comprises a polypeptide having, e.g., at least 1, 2, 3, 4, 5, 6, 7, 8, 9, 10, 20, 30, 40, 50, or 100 non-contiguous amino acid deletions, additions and/or substitutions relative to amino acids 1-445 of SEQ ID NO: 4; or at most 1, 2, 3, 4, 5, 6, 7, 8, 9, 10, 20, 30, 40, 50, or 100 non-contiguous amino acid substitutions relative to amino acids 1-445 of SEQ ID NO: 4. In still other aspects of this embodiment, a BoNT/D enzymatic domain comprises a polypeptide having, e.g., at least 1, 2, 3, 4, 5, 6, 7, 8, 9, 10, 20, 30, 40, 50, or 100 contiguous amino acid deletions, additions and/or substitutions relative to amino acids 1-445 of SEQ ID NO: 4; or at most 1, 2, 3, 4, 5, 6, 7, 8, 9, 10, 20, 30, 40, 50, or 100 contiguous amino acid substitutions relative to amino acids 1-445 of SEQ ID NO: 4.

In another embodiment, a Clostridial toxin enzymatic domain comprises a BoNT/E enzymatic domain. In an aspect of this embodiment, a BoNT/E enzymatic domain comprises amino acids 1-422 of SEQ ID NO: 5. In another aspect of this embodiment, a BoNT/E enzymatic domain comprises a naturally occurring BoNT/E enzymatic domain variant, such as, e.g., an enzymatic domain from a BoNT/E isoform or an enzymatic domain from a BoNT/E subtype. In another aspect of this embodiment, a BoNT/E enzymatic domain comprises amino acids 1-422 of a naturally occurring BoNT/E enzymatic domain variant of SEQ ID NO: 5, such as, e.g., amino acids 1-422 of a BoNT/E isoform of SEQ ID NO: 5 or amino acids 1-422 of a BoNT/E subtype of SEQ ID NO: 5. In still another aspect of this embodiment, a BoNT/E enzymatic domain comprises a non-naturally occurring BoNT/E enzymatic domain variant, such as, e.g., a conservative BoNT/E enzymatic domain variant, a non-conservative BoNT/E enzymatic domain variant, a BoNT/E chimeric enzymatic domain, an active BoNT/E enzymatic domain fragment, or any combination thereof. In still another aspect of this embodiment, a BoNT/E enzymatic domain comprises amino acids 1-422 of a non-naturally occurring BoNT/E enzymatic domain variant of SEQ ID NO: 5, such as, e.g., amino acids 1-422 of a conservative BoNT/E enzymatic domain variant of SEQ ID NO: 5, amino acids 1-422 of a non-conservative BoNT/E enzymatic domain variant of SEQ ID NO: 5, amino acids 1-422 of an active BoNT/E enzymatic domain fragment of SEQ ID NO: 5, or any combination thereof.

In other aspects of this embodiment, a BoNT/E enzymatic domain comprises a polypeptide having an amino acid identity of, e.g., at least 70%, at least 75%, at least 80%, at least 85%, at least 90%, at least 95%, or at least 97% to amino acids 1-422 of SEQ ID NO: 5; or at most 70%, at most 75%, at most 80%, at most 85%, at most 90%, at most 95%, or at most 97% to amino acids 1-422 of SEQ ID NO: 5. In yet other aspects of this embodiment, a BoNT/E enzymatic domain comprises a polypeptide having, e.g., at least 1, 2, 3, 4, 5, 6, 7, 8, 9, 10, 20, 30, 40, 50, or 100 non-contiguous amino acid deletions, additions and/or substitutions relative to amino acids 1-422 of SEQ ID NO: 5; or at most 1, 2, 3, 4, 5, 6, 7, 8, 9, 10, 20, 30, 40, 50, or 100 non-contiguous amino acid deletions, additions and/or substitutions relative to amino acids 1-422 of SEQ ID NO: 5. In still other aspects of this embodiment, a BoNT/E enzymatic domain comprises a polypeptide having, e.g., at least 1, 2, 3, 4, 5, 6, 7, 8, 9, 10, 20, 30, 40, 50, or 100 contiguous amino acid deletions, additions and/or substitutions relative to amino acids 1-422 of SEQ ID NO: 5; or at most 1, 2, 3, 4, 5, 6, 7, 8, 9, 10, 20, 30, 40, 50, or 100 contiguous amino acid deletions, additions and/or substitutions relative to amino acids 1-422 of SEQ ID NO: 5.

In another embodiment, a Clostridial toxin enzymatic domain comprises a BoNT/F enzymatic domain. In an aspect of this embodiment, a BoNT/F enzymatic domain comprises amino acids 1-439 of SEQ ID NO: 6. In another aspect of this embodiment, a BoNT/F enzymatic domain comprises a naturally occurring BoNT/F enzymatic domain variant, such as, e.g., an enzymatic domain from a BoNT/F isoform or an enzymatic domain from a BoNT/F subtype. In another aspect of this embodiment, a BoNT/F enzymatic domain comprises amino acids 1-439 of a naturally occurring BoNT/F enzymatic domain variant of SEQ ID NO: 6, such as, e.g., amino acids 1-439 of a BoNT/F isoform of SEQ ID NO: 6 or amino acids 1-439 of a BoNT/F subtype of SEQ ID NO: 6. In still another aspect of this embodiment, a BoNT/F enzymatic domain comprises a non-naturally occurring BoNT/F enzymatic domain variant, such as, e.g., a conservative BoNT/F enzymatic domain variant, a non-conservative BoNT/F enzymatic domain variant, a BoNT/F chimeric enzymatic domain, an active BoNT/F enzymatic domain fragment, or any combination thereof. In still another aspect of this embodiment, a BoNT/F enzymatic domain comprises amino acids 1-439 of a non-naturally occurring BoNT/F enzymatic domain variant of SEQ ID NO: 6, such as, e.g., amino acids 1-439 of a conservative BoNT/F enzymatic domain variant of SEQ ID NO: 6, amino acids 1-439 of a non-conservative BoNT/F enzymatic domain variant of SEQ ID NO: 6, amino acids 1-439 of an active BoNT/F enzymatic domain fragment of SEQ ID NO: 6, or any combination thereof.

In other aspects of this embodiment, a BoNT/F enzymatic domain comprises a polypeptide having an amino acid identity of, e.g., at least 70%, at least 75%, at least 80%, at least 85%, at least 90%, at least 95%, or at least 97% to amino acids 1-439 of SEQ ID NO: 6; or at most 70%, at most 75%, at most 80%, at most 85%, at most 90%, at most 95%, or at most 97% to amino acids 1-439 of SEQ ID NO: 6. In yet other aspects of this embodiment, a BoNT/F enzymatic domain comprises a polypeptide having, e.g., at least 1, 2, 3, 4, 5, 6, 7, 8, 9, 10, 20, 30, 40, 50, or 100 non-contiguous amino acid deletions, additions and/or substitutions relative to amino acids 1-439 of SEQ ID NO: 6; or at most 1, 2, 3, 4, 5, 6, 7, 8, 9, 10, 20, 30, 40, 50, or 100 non-contiguous amino acid deletions, additions and/or substitutions relative to amino acids 1-439 of SEQ ID NO: 6. In still other aspects of this embodiment, a BoNT/F enzymatic domain comprises a polypeptide having, e.g., at least 1, 2, 3, 4, 5, 6, 7, 8, 9, 10, 20, 30, 40, 50, or 100 contiguous amino acid deletions, additions and/or substitutions relative to amino acids 1-439 of SEQ ID NO: 6; or at most 1, 2, 3, 4, 5, 6, 7, 8, 9, 10, 20, 30, 40, 50, or 100 contiguous amino acid deletions, additions and/or substitutions relative to amino acids 1-439 of SEQ ID NO: 6.

In another embodiment, a Clostridial toxin enzymatic domain comprises a BoNT/G enzymatic domain. In an aspect of this embodiment, a BoNT/G enzymatic domain comprises amino acids 1-446 of SEQ ID NO: 7. In another aspect of this embodiment, a BoNT/G enzymatic domain comprises a naturally occurring BoNT/G enzymatic domain variant, such as, e.g., an enzymatic domain from a BoNT/G isoform or an enzymatic domain from a BoNT/G subtype. In another aspect of this embodiment, a BoNT/G enzymatic domain comprises amino acids 1-446 of a naturally occurring BoNT/G enzymatic domain variant of SEQ ID NO: 7, such as, e.g., amino acids 1-446 of a BoNT/G isoform of SEQ ID NO: 7 or amino acids 1-446 of a BoNT/G subtype of SEQ ID NO: 7. In still another aspect of this embodiment, a BoNT/G enzymatic domain comprises a non-naturally occurring BoNT/G enzymatic domain variant, such as, e.g., a conservative BoNT/G enzymatic domain variant, a non-conservative BoNT/G enzymatic domain variant, a BoNT/G chimeric enzymatic domain, an active BoNT/G enzymatic domain fragment, or any combination thereof. In still another aspect of this embodiment, a BoNT/G enzymatic domain comprises amino acids 1-446 of a non-naturally occurring BoNT/G enzymatic domain variant of SEQ ID NO: 7, such as, e.g., amino acids 1-446 of a conservative BoNT/G enzymatic domain variant of SEQ ID NO: 7, amino acids 1-446 of a non-conservative BoNT/G enzymatic domain variant of SEQ ID NO: 7, amino acids 1-446 of an active BoNT/G enzymatic domain fragment of SEQ ID NO: 7, or any combination thereof.

In other aspects of this embodiment, a BoNT/G enzymatic domain comprises a polypeptide having an amino acid identity of, e.g., at least 70%, at least 75%, at least 80%, at least 85%, at least 90%, at least 95%, or at least 97% to amino acids 1-446 of SEQ ID NO: 7; or at most 70%, at most 75%, at most 80%, at most 85%, at most 90%, at most 95%, or at most 97% to amino acids 1-446 of SEQ ID NO: 7. In yet other aspects of this embodiment, a BoNT/G enzymatic domain comprises a polypeptide having, e.g., at least 1, 2, 3, 4, 5, 6, 7, 8, 9, 10, 20, 30, 40, 50, or 100 non-contiguous amino acid deletions, additions and/or substitutions relative to amino acids 1-446 of SEQ ID NO: 7; or at most 1, 2, 3, 4, 5, 6, 7, 8, 9, 10, 20, 30, 40, 50, or 100 non-contiguous amino acid deletions, additions and/or substitutions relative to amino acids 1-446 of SEQ ID NO: 7. In still other aspects of this embodiment, a BoNT/G enzymatic domain comprises a polypeptide having, e.g., at least 1, 2, 3, 4, 5, 6, 7, 8, 9, 10, 20, 30, 40, 50, or 100 contiguous amino acid deletions, additions and/or substitutions relative to amino acids 1-446 of SEQ ID NO: 7; or at most 1, 2, 3, 4, 5, 6, 7, 8, 9, 10, 20, 30, 40, 50, or 100 contiguous amino acid deletions, additions and/or substitutions relative to amino acids 1-446 of SEQ ID NO: 7.

In another embodiment, a Clostridial toxin enzymatic domain comprises a TeNT enzymatic domain. In an aspect of this embodiment, a TeNT enzymatic domain comprises amino acids 1-457 of SEQ ID NO: 8. In another aspect of this embodiment, a TeNT enzymatic domain comprises a naturally occurring TeNT enzymatic domain variant, such as, e.g., an enzymatic domain from a TeNT isoform or an enzymatic domain from a TeNT subtype. In another aspect of this embodiment, a TeNT enzymatic domain comprises amino acids 1-457 of a naturally occurring TeNT enzymatic domain variant of SEQ ID NO: 8, such as, e.g., amino acids 1-457 of a TeNT isoform of SEQ ID NO: 8 or amino acids 1-457 of a TeNT subtype of SEQ ID NO: 8. In still another aspect of this embodiment, a TeNT enzymatic domain comprises a non-naturally occurring TeNT enzymatic domain variant, such as, e.g., a conservative TeNT enzymatic domain variant, a non-conservative TeNT enzymatic domain variant, a TeNT chimeric enzymatic domain, an active TeNT enzymatic domain fragment, or any combination thereof. In still another aspect of this embodiment, a TeNT enzymatic domain comprises amino acids 1-457 of a non-naturally occurring TeNT enzymatic domain variant of SEQ ID NO: 8, such as, e.g., amino acids 1-457 of a conservative TeNT enzymatic domain variant of SEQ ID NO: 8, amino acids 1-457 of a non-conservative TeNT enzymatic domain variant of SEQ ID NO: 8, amino acids 1-457 of an active TeNT enzymatic domain fragment of SEQ ID NO: 8, or any combination thereof.

In other aspects of this embodiment, a TeNT enzymatic domain comprises a polypeptide having an amino acid identity of, e.g., at least 70%, at least 75%, at least 80%, at least 85%, at least 90%, at least 95%, or at least 97% to amino acids 1-457 of SEQ ID NO: 8; or at most 70%, at most 75%, at most 80%, at most 85%, at most 90%, at most 95%, or at most 97% to amino acids 1-457 of SEQ ID NO: 8. In yet other aspects of this embodiment, a TeNT enzymatic domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 1-457 of SEQ ID NO: 8; or at most 1, 2, 3, 4, 5, 6, 7, 8, 9, 10, 20, 30, 40, 50, or 100 non-contiguous amino acid deletions, additions, and/or substitutions relative to amino acids 1-457 of SEQ ID NO: 8. In still other aspects of this embodiment, a TeNT enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 1-457 of SEQ ID NO: 8; at most 1, 2, 3, 4, 5, 6, 7, 8, 9, 10, 20, 30, 40, 50, or 100 contiguous amino acid substitutions relative to amino acids 1-457 of SEQ ID NO: 8.

In another embodiment, a Clostridial toxin enzymatic domain comprises a BaNT enzymatic domain. In an aspect of this embodiment, a BaNT enzymatic domain comprises amino acids 1-431 of SEQ ID NO: 9. In another aspect of this embodiment, a BaNT enzymatic domain comprises a naturally occurring BaNT enzymatic domain variant, such as, e.g., an enzymatic domain from a BaNT isoform or an enzymatic domain from a BaNT subtype. In another aspect of this embodiment, a BaNT enzymatic domain comprises amino acids 1-431 of a naturally occurring BaNT enzymatic domain variant of SEQ ID NO: 9, such as, e.g., amino acids 1-431 of a BaNT isoform of SEQ ID NO: 9 or amino acids 1-431 of a BaNT subtype of SEQ ID NO: 9. In still another aspect of this embodiment, a BaNT enzymatic domain comprises a non-naturally occurring BaNT enzymatic domain variant, such as, e.g., a conservative BaNT enzymatic domain variant, a non-conservative BaNT enzymatic domain variant, a BaNT chimeric enzymatic domain, an active BaNT enzymatic domain fragment, or any combination thereof. In still another aspect of this embodiment, a BaNT enzymatic domain comprises amino acids 1-431 of a non-naturally occurring BaNT enzymatic domain variant of SEQ ID NO: 9, such as, e.g., amino acids 1-431 of a conservative BaNT enzymatic domain variant of SEQ ID NO: 9, amino acids 1-431 of a non-conservative BaNT enzymatic domain variant of SEQ ID NO: 9, amino acids 1-431 of an active BaNT enzymatic domain fragment of SEQ ID NO: 9, or any combination thereof.

In other aspects of this embodiment, a BaNT enzymatic domain comprises a polypeptide having an amino acid identity of, e.g., at least 70%, at least 75%, at least 80%, at least 85%, at least 90%, at least 95%, or at least 97% to amino acids 1-431 of SEQ ID NO: 9; or at most 70%, at most 75%, at most 80%, at most 85%, at most 90%, at most 95%, or at most 97% to amino acids 1-431 of SEQ ID NO: 9. In yet other aspects of this embodiment, a BaNT enzymatic domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 1-431 of SEQ ID NO: 9; or at most 1, 2, 3, 4, 5, 6, 7, 8, 9, 10, 20, 30, 40, 50, or 100 non-contiguous amino acid deletions, additions, and/or substitutions relative to amino acids 1-431 of SEQ ID NO: 9. In still other aspects of this embodiment, a BaNT enzymatic domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 1-431 of SEQ ID NO: 9; at most 1, 2, 3, 4, 5, 6, 7, 8, 9, 10, 20, 30, 40, 50, or 100 contiguous amino acid deletions, additions, and/or substitutions relative to amino acids 1-431 of SEQ ID NO: 9.

In another embodiment, a Clostridial toxin enzymatic domain comprises a BuNT enzymatic domain. In an aspect of this embodiment, a BuNT enzymatic domain comprises amino acids 1-422 of SEQ ID NO: 10. In another aspect of this embodiment, a BuNT enzymatic domain comprises a naturally occurring BuNT enzymatic domain variant, such as, e.g., an enzymatic domain from a BuNT isoform or an enzymatic domain from a BuNT subtype. In another aspect of this embodiment, a BuNT enzymatic domain comprises amino acids 1-422 of a naturally occurring BuNT enzymatic domain variant of SEQ ID NO: 10, such as, e.g., amino acids 1-422 of a BuNT isoform of SEQ ID NO: 10 or amino acids 1-422 of a BuNT subtype of SEQ ID NO: 10. In still another aspect of this embodiment, a BuNT enzymatic domain comprises a non-naturally occurring BuNT enzymatic domain variant, such as, e.g., a conservative BuNT enzymatic domain variant, a non-conservative BuNT enzymatic domain variant, a BuNT chimeric enzymatic domain, an active BuNT enzymatic domain fragment, or any combination thereof. In still another aspect of this embodiment, a BuNT enzymatic domain comprises amino acids 1-422 of a non-naturally occurring BuNT enzymatic domain variant of SEQ ID NO: 10, such as, e.g., amino acids 1-422 of a conservative BuNT enzymatic domain variant of SEQ ID NO: 10, amino acids 1-422 of a non-conservative BuNT enzymatic domain variant of SEQ ID NO: 10, amino acids 1-422 of an active BuNT enzymatic domain fragment of SEQ ID NO: 10, or any combination thereof.

In other aspects of this embodiment, a BuNT enzymatic domain comprises a polypeptide having an amino acid identity of, e.g., at least 70%, at least 75%, at least 80%, at least 85%, at least 90%, at least 95%, or at least 97% to amino acids 1-422 of SEQ ID NO: 10; or at most 70%, at most 75%, at most 80%, at most 85%, at most 90%, at most 95%, or at most 97% to amino acids 1-422 of SEQ ID NO: 10. In yet other aspects of this embodiment, a BuNT enzymatic domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 1-422 of SEQ ID NO: 1; or at most 1, 2, 3, 4, 5, 6, 7, 8, 9, 10, 20, 30, 40, 50, or 100 non-contiguous amino acid deletions, additions, and/or substitutions relative to amino acids 1-422 of SEQ ID NO: 10. In still other aspects of this embodiment, a BuNT enzymatic domain comprises a polypeptide having, e.g., at least 1, 2, 3, 4, 5, 6, 7, 8, 9, 10, 20, 30, 40, 50, 100 or 200 contiguous amino acid deletions, additions, and/or substitutions relative to amino acids 1-422 of SEQ ID NO: 10; or at most 1, 2, 3, 4, 5, 6, 7, 8, 9, 10, 20, 30, 40, 50, 100 or 200 contiguous amino acid deletions, additions, and/or substitutions relative to amino acids 1-422 of SEQ ID NO: 10.

The “translocation domain” comprises a portion of a Clostridial neurotoxin heavy chain having a translocation activity. By “translocation” is meant the ability to facilitate the transport of a polypeptide through a vesicular membrane, thereby exposing some or all of the polypeptide to the cytoplasm. In the various botulinum neurotoxins translocation is thought to involve an allosteric conformational change of the heavy chain caused by a decrease in pH within the endosome. This conformational change appears to involve and be mediated by the N terminal half of the heavy chain and to result in the formation of pores in the vesicular membrane; this change permits the movement of the proteolytic light chain from within the endosomal vesicle into the cytoplasm. See e.g., Lacy, et al., Nature Struct. Biol. 5:898-902 (October 1998).

The amino acid sequence of the translocation-mediating portion of the botulinum neurotoxin heavy chain is known to those of skill in the art; additionally, those amino acid residues within this portion that are known to be essential for conferring the translocation activity are also known. It would therefore be well within the ability of one of ordinary skill in the art, for example, to employ the naturally occurring N-terminal peptide half of the heavy chain of any of the various Clostridium tetanus or Clostridium botulinum neurotoxin subtypes as a translocation domain, or to design an analogous translocation domain by aligning the primary sequences of the N-terminal halves of the various heavy chains and selecting a consensus primary translocation sequence based on conserved amino acid, polarity, steric and hydrophobicity characteristics between the sequences.

In another aspect of the invention, a TVEMP comprises, in part, a Clostridial toxin translocation domain. As used herein, the term “Clostridial toxin translocation domain” refers to any Clostridial toxin polypeptide that can execute the translocation step of the intoxication process that mediates Clostridial toxin light chain translocation. Thus, a Clostridial toxin translocation domain facilitates the movement of a Clostridial toxin light chain across a membrane and encompasses the movement of a Clostridial toxin light chain through the membrane an intracellular vesicle into the cytoplasm of a cell. Non-limiting examples of a Clostridial toxin translocation domain include, e.g., a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, and a BuNT translocation domain. Other non-limiting examples of a Clostridial toxin translocation domain include, e.g., amino acids 449-873 of SEQ ID NO: 1, amino acids 442-860 of SEQ ID NO: 2, amino acids 450-868 of SEQ ID NO: 3, amino acids 446-864 of SEQ ID NO: 4, amino acids 423-847 of SEQ ID NO: 5, amino acids 440-866 of SEQ ID NO: 6, amino acids 447-865 of SEQ ID NO: 7, amino acids 458-881 of SEQ ID NO: 8, amino acids 432-857 of SEQ ID NO: 9, and amino acids 423-847 of SEQ ID NO: 10.

A Clostridial toxin translocation domain includes, without limitation, naturally occurring Clostridial toxin translocation domain variants, such as, e.g., Clostridial toxin translocation domain isoforms and Clostridial toxin translocation domain subtypes; non-naturally occurring Clostridial toxin translocation domain variants, such as, e.g., conservative Clostridial toxin translocation domain variants, non-conservative Clostridial toxin translocation domain variants, Clostridial toxin translocation domain chimerics, active Clostridial toxin translocation domain fragments thereof, or any combination thereof.

As used herein, the term “Clostridial toxin translocation domain variant,” whether naturally-occurring or non-naturally-occurring, refers to a Clostridial toxin translocation domain that has at least one amino acid change from the corresponding region of the disclosed reference sequences (Table 1) and can be described in percent identity to the corresponding region of that reference sequence. Unless expressly indicated, Clostridial toxin translocation domain variants useful to practice disclosed embodiments are variants that execute the translocation step of the intoxication process that mediates Clostridial toxin light chain translocation. As non-limiting examples, a BoNT/A translocation domain variant comprising amino acids 449-873 of SEQ ID NO: 1 will have at least one amino acid difference, such as, e.g., an amino acid substitution, deletion or addition, as compared to the amino acid region 449-873 of SEQ ID NO: 1; a BoNT/B translocation domain variant comprising amino acids 442-860 of SEQ ID NO: 2 will have at least one amino acid difference, such as, e.g., an amino acid substitution, deletion or addition, as compared to the amino acid region 442-860 of SEQ ID NO: 2; a BoNT/C1 translocation domain variant comprising amino acids 450-868 of SEQ ID NO: 3 will have at least one amino acid difference, such as, e.g., an amino acid substitution, deletion or addition, as compared to the amino acid region 450-868 of SEQ ID NO: 3; a BoNT/D translocation domain variant comprising amino acids 446-864 of SEQ ID NO: 4 will have at least one amino acid difference, such as, e.g., an amino acid substitution, deletion or addition, as compared to the amino acid region 446-864 of SEQ ID NO: 4; a BoNT/E translocation domain variant comprising amino acids 423-847 of SEQ ID NO: 5 will have at least one amino acid difference, such as, e.g., an amino acid substitution, deletion or addition, as compared to the amino acid region 423-847 of SEQ ID NO: 5; a BoNT/F translocation domain variant comprising amino acids 440-866 of SEQ ID NO: 6 will have at least one amino acid difference, such as, e.g., an amino acid substitution, deletion or addition, as compared to the amino acid region 440-866 of SEQ ID NO: 6; a BoNT/G translocation domain variant comprising amino acids 447-865 of SEQ ID NO: 7 will have at least one amino acid difference, such as, e.g., an amino acid substitution, deletion or addition, as compared to the amino acid region 447-865 of SEQ ID NO: 7; a TeNT translocation domain variant comprising amino acids 458-881 of SEQ ID NO: 8 will have at least one amino acid difference, such as, e.g., an amino acid substitution, deletion or addition, as compared to the amino acid region 458-881 of SEQ ID NO: 8; a BaNT translocation domain variant comprising amino acids 432-857 of SEQ ID NO: 9 will have at least one amino acid difference, such as, e.g., an amino acid substitution, deletion or addition, as compared to the amino acid region 432-857 of SEQ ID NO: 9; and a BuNT translocation domain variant comprising amino acids 423-847 of SEQ ID NO: 10 will have at least one amino acid difference, such as, e.g., an amino acid substitution, deletion or addition, as compared to the amino acid region 423-847 of SEQ ID NO: 10.

It is recognized by those of skill in the art that within each serotype of Clostridial toxin there can be naturally occurring Clostridial toxin translocation domain variants that differ somewhat in their amino acid sequence, and also in the nucleic acids encoding these proteins. For example, there are presently five BoNT/A subtypes, BoNT/A1, BoNT/A2, BoNT/A3, BoNT/A4, and BoNT/A5, with specific translocation domain subtypes showing approximately 87% amino acid identity when compared to another BoNT/A translocation domain subtype. As used herein, the term “naturally occurring Clostridial toxin translocation domain variant” refers to any Clostridial toxin translocation domain produced by a naturally-occurring process, including, without limitation, Clostridial toxin translocation domain isoforms produced from alternatively-spliced transcripts, Clostridial toxin translocation domain isoforms produced by spontaneous mutation and Clostridial toxin translocation domain subtypes. A naturally occurring Clostridial toxin translocation domain variant can function in substantially the same manner as the reference Clostridial toxin translocation domain on which the naturally occurring Clostridial toxin translocation domain variant is based, and can be substituted for the reference Clostridial toxin translocation domain in any aspect of the present invention.

A non-limiting example of a naturally occurring Clostridial toxin translocation domain variant is a Clostridial toxin translocation domain isoform such as, e.g., a BoNT/A translocation domain isoform, a BoNT/B translocation domain isoform, a BoNT/C1 translocation domain isoform, a BoNT/D translocation domain isoform, a BoNT/E translocation domain isoform, a BoNT/F translocation domain isoform, a BoNT/G translocation domain isoform, a TeNT translocation domain isoform, a BaNT translocation domain isoform, and a BuNT translocation domain isoform. Another non-limiting example of a naturally occurring Clostridial toxin translocation domain variant is a Clostridial toxin translocation domain subtype such as, e.g., a translocation domain from subtype BoNT/A1, BoNT/A2, BoNT/A3, BoNT/A4, and BoNT/A5; a translocation domain from subtype BoNT/B1, BoNT/B2, BoNT/B bivalent and BoNT/B nonproteolytic; a translocation domain from subtype BoNT/C1-1 and BoNT/C1-2; a translocation domain from subtype BoNT/E1, BoNT/E2 and BoNT/E3; and a translocation domain from subtype BoNT/F1, BoNT/F2, BoNT/F3 and BoNT/F4.

As used herein, the term “non-naturally occurring Clostridial toxin translocation domain variant” refers to any Clostridial toxin translocation domain produced with the aid of human manipulation, including, without limitation, Clostridial toxin translocation domains produced by genetic engineering using random mutagenesis or rational design and Clostridial toxin translocation domains produced by chemical synthesis. Non-limiting examples of non-naturally occurring Clostridial toxin translocation domain variants include, e.g., conservative Clostridial toxin translocation domain variants, non-conservative Clostridial toxin translocation domain variants, Clostridial toxin translocation domain chimeric variants and active Clostridial toxin translocation domain fragments.

As used herein, the term “conservative Clostridial toxin translocation domain variant” refers to a Clostridial toxin translocation domain that has at least one amino acid substituted by another amino acid or an amino acid analog that has at least one property similar to that of the original amino acid from the reference Clostridial toxin translocation domain sequence (Table 1). Examples of properties include, without limitation, similar size, topography, charge, hydrophobicity, hydrophilicity, lipophilicity, covalent-bonding capacity, hydrogen-bonding capacity, a physicochemical property, of the like, or any combination thereof. A conservative Clostridial toxin translocation domain variant can function in substantially the same manner as the reference Clostridial toxin translocation domain on which the conservative Clostridial toxin translocation domain variant is based, and can be substituted for the reference Clostridial toxin translocation domain in any aspect of the present invention. Non-limiting examples of a conservative Clostridial toxin translocation domain variant include, e.g., conservative BoNT/A translocation domain variants, conservative BoNT/B translocation domain variants, conservative BoNT/C1 translocation domain variants, conservative BoNT/D translocation domain variants, conservative BoNT/E translocation domain variants, conservative BoNT/F translocation domain variants, conservative BoNT/G translocation domain variants, conservative TeNT translocation domain variants, conservative BaNT translocation domain variants, and conservative BuNT translocation domain variants.

As used herein, the term “non-conservative Clostridial toxin translocation domain variant” refers to a Clostridial toxin translocation domain in which 1) at least one amino acid is deleted from the reference Clostridial toxin translocation domain on which the non-conservative Clostridial toxin translocation domain variant is based; 2) at least one amino acid added to the reference Clostridial toxin translocation domain on which the non-conservative Clostridial toxin translocation domain is based; or 3) at least one amino acid is substituted by another amino acid or an amino acid analog that does not share any property similar to that of the original amino acid from the reference Clostridial toxin translocation domain sequence (Table 1). A non-conservative Clostridial toxin translocation domain variant can function in substantially the same manner as the reference Clostridial toxin translocation domain on which the non-conservative Clostridial toxin translocation domain variant is based, and can be substituted for the reference Clostridial toxin translocation domain in any aspect of the present invention. Non-limiting examples of a non-conservative Clostridial toxin translocation domain variant include, e.g., non-conservative BoNT/A translocation domain variants, non-conservative BoNT/B translocation domain variants, non-conservative BoNT/C1 translocation domain variants, non-conservative BoNT/D translocation domain variants, non-conservative BoNT/E translocation domain variants, non-conservative BoNT/F translocation domain variants, non-conservative BoNT/G translocation domain variants, and non-conservative TeNT translocation domain variants, non-conservative BaNT translocation domain variants, and non-conservative BuNT translocation domain variants.

As used herein, the term “Clostridial toxin translocation domain chimeric” refers to a polypeptide comprising at least a portion of a Clostridial toxin translocation domain and at least a portion of at least one other polypeptide to form a toxin translocation domain with at least one property different from the reference Clostridial toxin translocation domains of Table 1, with the proviso that this Clostridial toxin translocation domain chimeric is still capable of specifically targeting the core components of the neurotransmitter release apparatus and thus participate in executing the overall cellular mechanism whereby a Clostridial toxin proteolytically cleaves a substrate.

As used herein, the term “active Clostridial toxin translocation domain fragment” refers to any of a variety of Clostridial toxin fragments comprising the translocation domain can be useful in aspects of the present invention with the proviso that these active fragments can facilitate the release of the LC from intracellular vesicles into the cytoplasm of the target cell and thus participate in executing the overall cellular mechanism whereby a Clostridial toxin proteolytically cleaves a substrate. The translocation domains from the heavy chains of Clostridial toxins are approximately 410-430 amino acids in length and comprise a translocation domain (Table 1). Research has shown that the entire length of a translocation domain from a Clostridial toxin heavy chain is not necessary for the translocating activity of the translocation domain. Thus, aspects of this embodiment can include Clostridial toxin translocation domains comprising a translocation domain having a length of, e.g., at least 350 amino acids, at least 375 amino acids, at least 400 amino acids or at least 425 amino acids. Other aspects of this embodiment can include Clostridial toxin translocation domains comprising translocation domain having a length of, e.g., at most 350 amino acids, at most 375 amino acids, at most 400 amino acids or at most 425 amino acids.

Any of a variety of sequence alignment methods can be used to determine percent identity of naturally-occurring Clostridial toxin translocation domain variants and non-naturally-occurring Clostridial toxin translocation domain variants, including, without limitation, global methods, local methods and hybrid methods, such as, e.g., segment approach methods. Protocols to determine percent identity are routine procedures within the scope of one skilled in the art and from the teaching herein.

Thus, in an embodiment, a TVEMP disclosed in the present specification comprises a Clostridial toxin translocation domain. In an aspect of this embodiment, a Clostridial toxin translocation domain comprises a naturally occurring Clostridial toxin translocation domain variant, such as, e.g., a Clostridial toxin translocation domain isoform or a Clostridial toxin translocation domain subtype. In another aspect of this embodiment, a Clostridial toxin translocation domain comprises a non-naturally occurring Clostridial toxin translocation domain variant, such as, e.g., a conservative Clostridial toxin translocation domain variant, a non-conservative Clostridial toxin translocation domain variant, a Clostridial toxin chimeric translocation domain, an active Clostridial toxin translocation domain fragment, or any combination thereof.

In another embodiment, a Clostridial toxin translocation domain comprises a BoNT/A translocation domain. In an aspect of this embodiment, a BoNT/A translocation domain comprises amino acids 449-873 of SEQ ID NO: 1. In another aspect of this embodiment, a BoNT/A translocation domain comprises a naturally occurring BoNT/A translocation domain variant, such as, e.g., a translocation domain from a BoNT/A isoform or a translocation domain from a BoNT/A subtype. In another aspect of this embodiment, a BoNT/A translocation domain comprises amino acids 449-873 of a naturally occurring BoNT/A translocation domain variant of SEQ ID NO: 1, such as, e.g., amino acids 449-873 of a BoNT/A isoform of SEQ ID NO: 1 or amino acids 449-873 of a BoNT/A subtype of SEQ ID NO: 1. In still another aspect of this embodiment, a BoNT/A translocation domain comprises a non-naturally occurring BoNT/A translocation domain variant, such as, e.g., a conservative BoNT/A translocation domain variant, a non-conservative BoNT/A translocation domain variant, a BoNT/A chimeric translocation domain, an active BoNT/A translocation domain fragment, or any combination thereof. In still another aspect of this embodiment, a BoNT/A translocation domain comprises amino acids 449-873 of a non-naturally occurring BoNT/A translocation domain variant of SEQ ID NO: 1, such as, e.g., amino acids 449-873 of a conservative BoNT/A translocation domain variant of SEQ ID NO: 1, amino acids 449-873 of a non-conservative BoNT/A translocation domain variant of SEQ ID NO: 1, amino acids 449-873 of an active BoNT/A translocation domain fragment of SEQ ID NO: 1, or any combination thereof.

In other aspects of this embodiment, a BoNT/A translocation domain comprises a polypeptide having an amino acid identity of, e.g., at least 70%, at least 75%, at least 80%, at least 85%, at least 90%, at least 95%, or at least 97% to amino acids 449-873 of SEQ ID NO: 1; or at most 70%, at most 75%, at most 80%, at most 85%, at most 90%, at most 95%, or at most 97% to amino acids 449-873 of SEQ ID NO: 1. In yet other aspects of this embodiment, a BoNT/A translocation domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 449-873 of SEQ ID NO: 1; at most 1, 2, 3, 4, 5, 6, 7, 8, 9, 10, 20, 30, 40, 50, or 100 non-contiguous amino acid deletions, additions, and/or substitutions relative to amino acids 449-873 of SEQ ID NO: 1. In still other aspects of this embodiment, a BoNT/A translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 449-873 of SEQ ID NO: 1; at most 1, 2, 3, 4, 5, 6, 7, 8, 9, 10, 20, 30, 40, 50, 100 or 200 contiguous amino acid deletions, additions, and/or substitutions relative to amino acids 449-873 of SEQ ID NO: 1.

In another embodiment, a Clostridial toxin translocation domain comprises a BoNT/B translocation domain. In an aspect of this embodiment, a BoNT/B translocation domain comprises amino acids 442-860 of SEQ ID NO: 2. In another aspect of this embodiment, a BoNT/B translocation domain comprises a naturally occurring BoNT/B translocation domain variant, such as, e.g., a translocation domain from a BoNT/B isoform or a translocation domain from a BoNT/B subtype. In another aspect of this embodiment, a BoNT/B translocation domain comprises amino acids 442-860 of a naturally occurring BoNT/B translocation domain variant of SEQ ID NO: 2, such as, e.g., amino acids 442-860 of a BoNT/B isoform of SEQ ID NO: 2 or amino acids 442-860 of a BoNT/B subtype of SEQ ID NO: 2. In still another aspect of this embodiment, a BoNT/B translocation domain comprises a non-naturally occurring BoNT/B translocation domain variant, such as, e.g., a conservative BoNT/B translocation domain variant, a non-conservative BoNT/B translocation domain variant, a BoNT/B chimeric translocation domain, an active BoNT/B translocation domain fragment, or any combination thereof. In still another aspect of this embodiment, a BoNT/B translocation domain comprises amino acids 442-860 of a non-naturally occurring BoNT/B translocation domain variant of SEQ ID NO: 2, such as, e.g., amino acids 442-860 of a conservative BoNT/B translocation domain variant of SEQ ID NO: 2, amino acids 442-860 of a non-conservative BoNT/B translocation domain variant of SEQ ID NO: 2, amino acids 442-860 of an active BoNT/B translocation domain fragment of SEQ ID NO: 2, or any combination thereof.

In other aspects of this embodiment, a BoNT/B translocation domain comprises a polypeptide having an amino acid identity of, e.g., at least 70%, at least 75%, at least 80%, at least 85%, at least 90%, at least 95%, or at least 97% to amino acids 442-860 of SEQ ID NO: 2; or at most 70%, at most 75%, at most 80%, at most 85%, at most 90%, at most 95%, or at most 97% to amino acids 442-860 of SEQ ID NO: 2. In yet other aspects of this embodiment, a BoNT/B translocation domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 442-860 of SEQ ID NO: 2; or at most 1, 2, 3, 4, 5, 6, 7, 8, 9, 10, 20, 30, 40, 50, 100 or 200 non-contiguous amino acid deletions, additions, and/or substitutions relative to amino acids 442-860 of SEQ ID NO: 2. In still other aspects of this embodiment, a BoNT/B translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 442-860 of SEQ ID NO: 2; or at most 1, 2, 3, 4, 5, 6, 7, 8, 9, 10, 20, 30, 40, 50, or 100 contiguous amino acid deletions, additions, and/or substitutions relative to amino acids 442-860 of SEQ ID NO: 2.

In another embodiment, a Clostridial toxin translocation domain comprises a BoNT/C1 translocation domain. In an aspect of this embodiment, a BoNT/C1 translocation domain comprises amino acids 450-868 of SEQ ID NO: 3. In another aspect of this embodiment, a BoNT/C1 translocation domain comprises a naturally occurring BoNT/C1 translocation domain variant, such as, e.g., a translocation domain from a BoNT/C1 isoform or a translocation domain from a BoNT/C1 subtype. In another aspect of this embodiment, a BoNT/C1 translocation domain comprises amino acids 450-868 of a naturally occurring BoNT/C1 translocation domain variant of SEQ ID NO: 3, such as, e.g., amino acids 450-868 of a BoNT/C1 isoform of SEQ ID NO: 3 or amino acids 450-868 of a BoNT/C1 subtype of SEQ ID NO: 3. In still another aspect of this embodiment, a BoNT/C1 translocation domain comprises a non-naturally occurring BoNT/C1 translocation domain variant, such as, e.g., a conservative BoNT/C1 translocation domain variant, a non-conservative BoNT/C1 translocation domain variant, a BoNT/C1 chimeric translocation domain, an active BoNT/C1 translocation domain fragment, or any combination thereof. In still another aspect of this embodiment, a BoNT/C1 translocation domain comprises amino acids 450-868 of a non-naturally occurring BoNT/C1 translocation domain variant of SEQ ID NO: 3, such as, e.g., amino acids 450-868 of a conservative BoNT/C1 translocation domain variant of SEQ ID NO: 3, amino acids 450-868 of a non-conservative BoNT/C1 translocation domain variant of SEQ ID NO: 3, amino acids 450-868 of an active BoNT/C1 translocation domain fragment of SEQ ID NO: 3, or any combination thereof.

In other aspects of this embodiment, a BoNT/C1 translocation domain comprises a polypeptide having an amino acid identity of, e.g., at least 70%, at least 75%, at least 80%, at least 85%, at least 90%, at least 95%, or at least 97% to amino acids 450-868 of SEQ ID NO: 3; or at most 70%, at most 75%, at most 80%, at most 85%, at most 90%, at most 95%, or at most 97% to amino acids 450-868 of SEQ ID NO: 3. In yet other aspects of this embodiment, a BoNT/C1 translocation domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 450-868 of SEQ ID NO: 3; or at most 1, 2, 3, 4, 5, 6, 7, 8, 9, 10, 20, 30, 40, 50, or 100 non-contiguous amino acid deletions, additions, and/or substitutions relative to amino acids 450-868 of SEQ ID NO: 3. In still other aspects of this embodiment, a BoNT/C1 translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 450-868 of SEQ ID NO: 3; or at most 1, 2, 3, 4, 5, 6, 7, 8, 9, 10, 20, 30, 40, 50, or 100 contiguous amino acid deletions, additions, and/or substitutions relative to amino acids 450-868 of SEQ ID NO: 3.

In another embodiment, a Clostridial toxin translocation domain comprises a BoNT/D translocation domain. In an aspect of this embodiment, a BoNT/D translocation domain comprises amino acids 446-864 of SEQ ID NO: 4. In another aspect of this embodiment, a BoNT/D translocation domain comprises a naturally occurring BoNT/D translocation domain variant, such as, e.g., a translocation domain from a BoNT/D isoform or a translocation domain from a BoNT/D subtype. In another aspect of this embodiment, a BoNT/D translocation domain comprises amino acids 446-864 of a naturally occurring BoNT/D translocation domain variant of SEQ ID NO: 4, such as, e.g., amino acids 446-864 of a BoNT/D isoform of SEQ ID NO: 4 or amino acids 446-864 of a BoNT/D subtype of SEQ ID NO: 4. In still another aspect of this embodiment, a BoNT/D translocation domain comprises a non-naturally occurring BoNT/D translocation domain variant, such as, e.g., a conservative BoNT/D translocation domain variant, a non-conservative BoNT/D translocation domain variant, a BoNT/D chimeric translocation domain, an active BoNT/D translocation domain fragment, or any combination thereof. In still another aspect of this embodiment, a BoNT/D translocation domain comprises amino acids 446-864 of a non-naturally occurring BoNT/D translocation domain variant of SEQ ID NO: 4, such as, e.g., amino acids 446-864 of a conservative BoNT/D translocation domain variant of SEQ ID NO: 4, amino acids 446-864 of a non-conservative BoNT/D translocation domain variant of SEQ ID NO: 4, amino acids 446-864 of an active BoNT/D translocation domain fragment of SEQ ID NO: 4, or any combination thereof.

In other aspects of this embodiment, a BoNT/D translocation domain comprises a polypeptide having an amino acid identity of, e.g., at least 70%, at least 75%, at least 80%, at least 85%, at least 90%, at least 95%, or at least 97% to amino acids 446-864 of SEQ ID NO: 4; or at most 70%, at most 75%, at most 80%, at most 85%, at most 90%, at most 95%, or at most 97% to amino acids 446-864 of SEQ ID NO: 4. In yet other aspects of this embodiment, a BoNT/D translocation domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 446-864 of SEQ ID NO: 4; or at most 1, 2, 3, 4, 5, 6, 7, 8, 9, 10, 20, 30, 40, 50, or 100 non-contiguous amino acid deletions, additions, and/or substitutions relative to amino acids 446-864 of SEQ ID NO: 4. In still other aspects of this embodiment, a BoNT/D translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 446-864 of SEQ ID NO: 4; or at most 1, 2, 3, 4, 5, 6, 7, 8, 9, 10, 20, 30, 40, 50, or 100 contiguous amino acid substitutions relative to amino acids 446-864 of SEQ ID NO: 4.

In another embodiment, a Clostridial toxin translocation domain comprises a BoNT/E translocation domain. In an aspect of this embodiment, a BoNT/E translocation domain comprises amino acids 423-847 of SEQ ID NO: 5. In another aspect of this embodiment, a BoNT/E translocation domain comprises a naturally occurring BoNT/E translocation domain variant, such as, e.g., a translocation domain from a BoNT/E isoform or a translocation domain from a BoNT/E subtype. In another aspect of this embodiment, a BoNT/E translocation domain comprises amino acids 423-847 of a naturally occurring BoNT/E translocation domain variant of SEQ ID NO: 5, such as, e.g., amino acids 423-847 of a BoNT/E isoform of SEQ ID NO: 5 or amino acids 423-847 of a BoNT/E subtype of SEQ ID NO: 5. In still another aspect of this embodiment, a BoNT/E translocation domain comprises a non-naturally occurring BoNT/E translocation domain variant, such as, e.g., a conservative BoNT/E translocation domain variant, a non-conservative BoNT/E translocation domain variant, a BoNT/E chimeric translocation domain, an active BoNT/E translocation domain fragment, or any combination thereof. In still another aspect of this embodiment, a BoNT/E translocation domain comprises amino acids 423-847 of a non-naturally occurring BoNT/E translocation domain variant of SEQ ID NO: 5, such as, e.g., amino acids 423-847 of a conservative BoNT/E translocation domain variant of SEQ ID NO: 5, amino acids 423-847 of a non-conservative BoNT/E translocation domain variant of SEQ ID NO: 5, amino acids 423-847 of an active BoNT/E translocation domain fragment of SEQ ID NO: 5, or any combination thereof.

In other aspects of this embodiment, a BoNT/E translocation domain comprises a polypeptide having an amino acid identity of, e.g., at least 70%, at least 75%, at least 80%, at least 85%, at least 90%, at least 95%, or at least 97% to amino acids 423-847 of SEQ ID NO: 5; or at most 70%, at most 75%, at most 80%, at most 85%, at most 90%, at most 95%, or at most 97% to amino acids 423-847 of SEQ ID NO: 5. In yet other aspects of this embodiment, a BoNT/E translocation domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 423-847 of SEQ ID NO: 5; or at most 1, 2, 3, 4, 5, 6, 7, 8, 9, 10, 20, 30, 40, 50, or 100 non-contiguous amino acid deletions, additions, and/or substitutions relative to amino acids 423-847 of SEQ ID NO: 5. In still other aspects of this embodiment, a BoNT/E translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 423-847 of SEQ ID NO: 5; or at most 1, 2, 3, 4, 5, 6, 7, 8, 9, 10, 20, 30, 40, 50, or 100 contiguous amino acid substitutions relative to amino acids 423-847 of SEQ ID NO: 5.

In another embodiment, a Clostridial toxin translocation domain comprises a BoNT/F translocation domain. In an aspect of this embodiment, a BoNT/F translocation domain comprises amino acids 440-866 of SEQ ID NO: 6. In another aspect of this embodiment, a BoNT/F translocation domain comprises a naturally occurring BoNT/F translocation domain variant, such as, e.g., a translocation domain from a BoNT/F isoform or a translocation domain from a BoNT/F subtype. In another aspect of this embodiment, a BoNT/F translocation domain comprises amino acids 440-866 of a naturally occurring BoNT/F translocation domain variant of SEQ ID NO: 6, such as, e.g., amino acids 440-866 of a BoNT/F isoform of SEQ ID NO: 6 or amino acids 440-866 of a BoNT/F subtype of SEQ ID NO: 6. In still another aspect of this embodiment, a BoNT/F translocation domain comprises a non-naturally occurring BoNT/F translocation domain variant, such as, e.g., a conservative BoNT/F translocation domain variant, a non-conservative BoNT/F translocation domain variant, a BoNT/F chimeric translocation domain, an active BoNT/F translocation domain fragment, or any combination thereof. In still another aspect of this embodiment, a BoNT/F translocation domain comprises amino acids 440-866 of a non-naturally occurring BoNT/F translocation domain variant of SEQ ID NO: 6, such as, e.g., amino acids 440-866 of a conservative BoNT/F translocation domain variant of SEQ ID NO: 6, amino acids 440-866 of a non-conservative BoNT/F translocation domain variant of SEQ ID NO: 6, amino acids 440-866 of an active BoNT/F translocation domain fragment of SEQ ID NO: 6, or any combination thereof.

In other aspects of this embodiment, a BoNT/F translocation domain comprises a polypeptide having an amino acid identity of, e.g., at least 70%, at least 75%, at least 80%, at least 85%, at least 90%, at least 95%, or at least 97% to amino acids 440-866 of SEQ ID NO: 6; or at most 70%, at most 75%, at most 80%, at most 85%, at most 90%, at most 95%, or at most 97% to amino acids 440-866 of SEQ ID NO: 6. In yet other aspects of this embodiment, a BoNT/F translocation domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 440-866 of SEQ ID NO: 6; or at most 1, 2, 3, 4, 5, 6, 7, 8, 9, 10, 20, 30, 40, 50, or 100 non-contiguous amino acid deletions, additions, and/or substitutions relative to amino acids 440-866 of SEQ ID NO: 6. In still other aspects of this embodiment, a BoNT/F translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 440-866 of SEQ ID NO: 6; or at most 1, 2, 3, 4, 5, 6, 7, 8, 9, 10, 20, 30, 40, 50, or 100 contiguous amino acid substitutions relative to amino acids 440-866 of SEQ ID NO: 6.

In another embodiment, a Clostridial toxin translocation domain comprises a BoNT/G translocation domain. In an aspect of this embodiment, a BoNT/G translocation domain comprises amino acids 447-865 of SEQ ID NO: 7. In another aspect of this embodiment, a BoNT/G translocation domain comprises a naturally occurring BoNT/G translocation domain variant, such as, e.g., a translocation domain from a BoNT/G isoform or a translocation domain from a BoNT/G subtype. In another aspect of this embodiment, a BoNT/G translocation domain comprises amino acids 447-865 of a naturally occurring BoNT/G translocation domain variant of SEQ ID NO: 7, such as, e.g., amino acids 447-865 of a BoNT/G isoform of SEQ ID NO: 7 or amino acids 447-865 of a BoNT/G subtype of SEQ ID NO: 7. In still another aspect of this embodiment, a BoNT/G translocation domain comprises a non-naturally occurring BoNT/G translocation domain variant, such as, e.g., a conservative BoNT/G translocation domain variant, a non-conservative BoNT/G translocation domain variant, a BoNT/G chimeric translocation domain, an active BoNT/G translocation domain fragment, or any combination thereof. In still another aspect of this embodiment, a BoNT/G translocation domain comprises amino acids 447-865 of a non-naturally occurring BoNT/G translocation domain variant of SEQ ID NO: 7, such as, e.g., amino acids 447-865 of a conservative BoNT/G translocation domain variant of SEQ ID NO: 7, amino acids 447-865 of a non-conservative BoNT/G translocation domain variant of SEQ ID NO: 7, amino acids 447-865 of an active BoNT/G translocation domain fragment of SEQ ID NO: 7, or any combination thereof.

In other aspects of this embodiment, a BoNT/G translocation domain comprises a polypeptide having an amino acid identity of, e.g., at least 70%, at least 75%, at least 80%, at least 85%, at least 90%, at least 95%, or at least 97% to amino acids 447-865 of SEQ ID NO: 7; or at most 70%, at most 75%, at most 80%, at most 85%, at most 90%, at most 95%, or at most 97% to amino acids 447-865 of SEQ ID NO: 7. In yet other aspects of this embodiment, a BoNT/G translocation domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 447-865 of SEQ ID NO: 7; or at most 1, 2, 3, 4, 5, 6, 7, 8, 9, 10, 20, 30, 40, 50, or 100 non-contiguous amino acid deletions, additions, and/or substitutions relative to amino acids 447-865 of SEQ ID NO: 7. In still other aspects of this embodiment, a BoNT/G translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 447-865 of SEQ ID NO: 7; or at most 1, 2, 3, 4, 5, 6, 7, 8, 9, 10, 20, 30, 40, 50, or 100 contiguous amino acid deletions, additions, and/or substitutions relative to amino acids 447-865 of SEQ ID NO: 7.

In another embodiment, a Clostridial toxin translocation domain comprises a TeNT translocation domain. In an aspect of this embodiment, a TeNT translocation domain comprises amino acids 458-881 of SEQ ID NO: 8. In another aspect of this embodiment, a TeNT translocation domain comprises a naturally occurring TeNT translocation domain variant, such as, e.g., a translocation domain from a TeNT isoform or a translocation domain from a TeNT subtype. In another aspect of this embodiment, a TeNT translocation domain comprises amino acids 458-881 of a naturally occurring TeNT translocation domain variant of SEQ ID NO: 8, such as, e.g., amino acids 458-881 of a TeNT isoform of SEQ ID NO: 8 or amino acids 458-881 of a TeNT subtype of SEQ ID NO: 8. In still another aspect of this embodiment, a TeNT translocation domain comprises a non-naturally occurring TeNT translocation domain variant, such as, e.g., a conservative TeNT translocation domain variant, a non-conservative TeNT translocation domain variant, a TeNT chimeric translocation domain, an active TeNT translocation domain fragment, or any combination thereof. In still another aspect of this embodiment, a TeNT translocation domain comprises amino acids 458-881 of a non-naturally occurring TeNT translocation domain variant of SEQ ID NO: 8, such as, e.g., amino acids 458-881 of a conservative TeNT translocation domain variant of SEQ ID NO: 8, amino acids 458-881 of a non-conservative TeNT translocation domain variant of SEQ ID NO: 8, amino acids 458-881 of an active TeNT translocation domain fragment of SEQ ID NO: 8, or any combination thereof.

In other aspects of this embodiment, a TeNT translocation domain comprises a polypeptide having an amino acid identity of, e.g., at least 70%, at least 75%, at least 80%, at least 85%, at least 90%, at least 95%, or at least 97% to amino acids 458-881 of SEQ ID NO: 8; or at most 70%, at most 75%, at most 80%, at most 85%, at most 90%, at most 95%, or at most 97% to amino acids 458-881 of SEQ ID NO: 8. In yet other aspects of this embodiment, a TeNT translocation domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 458-881 of SEQ ID NO: 8; or at most 1, 2, 3, 4, 5, 6, 7, 8, 9, 10, 20, 30, 40, 50, or 100 non-contiguous amino acid deletions, additions, and/or substitutions relative to amino acids 458-881 of SEQ ID NO: 8. In still other aspects of this embodiment, a TeNT translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 458-881 of SEQ ID NO: 8; or at most 1, 2, 3, 4, 5, 6, 7, 8, 9, 10, 20, 30, 40, 50, or 100 contiguous amino acid deletions, additions, and/or substitutions relative to amino acids 458-881 of SEQ ID NO: 8.

In another embodiment, a Clostridial toxin translocation domain comprises a BaNT translocation domain. In an aspect of this embodiment, a BaNT translocation domain comprises amino acids 432-857 of SEQ ID NO: 9. In another aspect of this embodiment, a BaNT translocation domain comprises a naturally occurring BaNT translocation domain variant, such as, e.g., a translocation domain from a BaNT isoform or a translocation domain from a BaNT subtype. In another aspect of this embodiment, a BaNT translocation domain comprises amino acids 432-857 of a naturally occurring BaNT translocation domain variant of SEQ ID NO: 9, such as, e.g., amino acids 432-857 of a BaNT isoform of SEQ ID NO: 9 or amino acids 432-857 of a BaNT subtype of SEQ ID NO: 9. In still another aspect of this embodiment, a BaNT translocation domain comprises a non-naturally occurring BaNT translocation domain variant, such as, e.g., a conservative BaNT translocation domain variant, a non-conservative BaNT translocation domain variant, a BaNT chimeric translocation domain, an active BaNT translocation domain fragment, or any combination thereof. In still another aspect of this embodiment, a BaNT translocation domain comprises amino acids 432-857 of a non-naturally occurring BaNT translocation domain variant of SEQ ID NO: 9, such as, e.g., amino acids 432-857 of a conservative BaNT translocation domain variant of SEQ ID NO: 9, amino acids 432-857 of a non-conservative BaNT translocation domain variant of SEQ ID NO: 9, amino acids 432-857 of an active BaNT translocation domain fragment of SEQ ID NO: 9, or any combination thereof.

In other aspects of this embodiment, a BaNT translocation domain comprises a polypeptide having an amino acid identity of, e.g., at least 70%, at least 75%, at least 80%, at least 85%, at least 90%, at least 95%, or at least 97% to amino acids 432-857 of SEQ ID NO: 9; or at most 70%, at most 75%, at most 80%, at most 85%, at most 90%, at most 95%, or at most 97% to amino acids 432-857 of SEQ ID NO: 9. In yet other aspects of this embodiment, a BaNT translocation domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 432-857 of SEQ ID NO: 9; or at most 1, 2, 3, 4, 5, 6, 7, 8, 9, 10, 20, 30, 40, 50, or 100 non-contiguous amino acid deletions, additions, and/or substitutions relative to amino acids 432-857 of SEQ ID NO: 9. In still other aspects of this embodiment, a BaNT translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 432-857 of SEQ ID NO: 9; or at most 1, 2, 3, 4, 5, 6, 7, 8, 9, 10, 20, 30, 40, 50, or 100 contiguous amino acid deletions, additions, and/or substitutions relative to amino acids 432-857 of SEQ ID NO: 9.

In another embodiment, a Clostridial toxin translocation domain comprises a BuNT translocation domain. In an aspect of this embodiment, a BuNT translocation domain comprises amino acids 423-847 of SEQ ID NO: 10. In another aspect of this embodiment, a BuNT translocation domain comprises a naturally occurring BuNT translocation domain variant, such as, e.g., a translocation domain from a BuNT isoform or a translocation domain from a BuNT subtype. In another aspect of this embodiment, a BuNT translocation domain comprises amino acids 423-847 of a naturally occurring BuNT translocation domain variant of SEQ ID NO: 10, such as, e.g., amino acids 423-847 of a BuNT isoform of SEQ ID NO: 10 or amino acids 423-847 of a BuNT subtype of SEQ ID NO: 10. In still another aspect of this embodiment, a BuNT translocation domain comprises a non-naturally occurring BuNT translocation domain variant, such as, e.g., a conservative BuNT translocation domain variant, a non-conservative BuNT translocation domain variant, a BuNT chimeric translocation domain, an active BuNT translocation domain fragment, or any combination thereof. In still another aspect of this embodiment, a BuNT translocation domain comprises amino acids 423-847 of a non-naturally occurring BuNT translocation domain variant of SEQ ID NO: 10, such as, e.g., amino acids 423-847 of a conservative BuNT translocation domain variant of SEQ ID NO: 10, amino acids 423-847 of a non-conservative BuNT translocation domain variant of SEQ ID NO: 10, amino acids 423-847 of an active BuNT translocation domain fragment of SEQ ID NO: 10, or any combination thereof.

In other aspects of this embodiment, a BuNT translocation domain comprises a polypeptide having an amino acid identity of, e.g., at least 70%, at least 75%, at least 80%, at least 85%, at least 90%, at least 95%, or at least 97% of amino acids 423-847 of SEQ ID NO: 10; or at most 70%, at most 75%, at most 80%, at most 85%, at most 90%, at most 95%, or at most 97% of amino acids 423-847 of SEQ ID NO: 10. In yet other aspects of this embodiment, a BuNT translocation domain comprises a polypeptide having, e.g., at least 1, 2, 3, 4, 5, 6, 7, 8, 9, 10, 20, 30, 40, 50, or 100 non-contiguous amino acid deletions, additions, and/or substitutions relative to amino acids 423-847 of SEQ ID NO: 10; or at most 1, 2, 3, 4, 5, 6, 7, 8, 9, 10, 20, 30, 40, 50, or 100 non-contiguous amino acid deletions, additions, and/or substitutions relative to amino acids 423-847 of SEQ ID NO: 10. In still other aspects of this embodiment, a BuNT translocation domain comprises a polypeptide having, e.g., at least 1, 2, 3, 4, 5, 6, 7, 8, 9, 10, 20, 30, 40, 50, or 100 contiguous amino acid deletions, additions, and/or substitutions relative to amino acids 423-847 of SEQ ID NO: 10; or at most 1, 2, 3, 4, 5, 6, 7, 8, 9, 10, 20, 30, 40, 50, or 100 contiguous amino acid deletions, additions, and/or substitutions relative to amino acids 423-847 of SEQ ID NO: 10.

In another aspect of the invention, a TVEMP comprises, in part, a retargeted peptide binding domain. As used herein, the term “peptide binding domain” refers to an amino acid sequence region able to selectively bind to a cell surface marker characteristic of the target cell under physiological conditions. As used herein, the term “retargeted peptide binding domain” refers to a peptide binding domain that does not selectively bind to a Clostridial toxin receptor under physiological conditions. The cell surface marker may comprise a polypeptide, a polysaccharide, a lipid, a glycoprotein, a lipoprotein, or may have structural characteristics of more than one of these. As used herein, the term “selectively bind” refers to molecule is able to bind its target receptor under physiological conditions, or in vitro conditions substantially approximating physiological conditions, to a statistically significantly greater degree relative to other, non-target receptors.

Thus, in an embodiment, a retargeted binding domain that selectively binds a target receptor has a dissociation equilibrium constant (K_{D}) that is greater for the target receptor relative to a non-target receptor by, e.g., at least one-fold, at least two-fold, at least three-fold, at least four fold, at least five-fold, at least 10 fold, at least 50 fold, at least 100 fold, at least 1000 fold, at least 10,000 fold, or at least 100,000 fold. In another embodiment, a retargeted binding domain that selectively binds a target receptor has a dissociation equilibrium constant (K_{D}) that is greater for the target receptor relative to a non-target receptor by, e.g., about one-fold to about three-fold, about one-fold to about five-fold, about one-fold to about 10-fold, about one-fold to about 100-fold, about one-fold to about 1000-fold, about five-fold to about 10-fold, about five-fold to about 100-fold, about five-fold to about 1000-fold, about 10-fold to about 100-fold, about 10-fold to about 1000-fold, about 10-fold to about 10,000-fold, or about 10-fold to about 100,000-fold.

An example of a retargeted binding element disclosed in the present specification is an interleukin (IL) peptide binding domain. Non-limiting examples of an IL peptide binding domain include an IL-1, an IL-2, an IL-6, an IL-8, an IL-10, or an IL-11.

Thus, in an embodiment, a retargeted binding domain comprises an IL peptide binding domain. In aspects of this embodiment, an IL peptide binding domain comprises an IL-1, an IL-2, an IL-6, an IL-8, an IL-10, or an IL-11. In other aspects of this embodiment, an IL peptide binding domain comprises SEQ ID NO: 67, SEQ ID NO: 68, SEQ ID NO: 69, SEQ ID NO: 70, SEQ ID NO: 71, or SEQ ID NO: 72. In yet other aspects of this embodiment, an IL peptide binding domain comprises amino acids 123-265 of SEQ ID NO: 67, amino acids 21-153 of SEQ ID NO: 68, amino acids 57-210 of SEQ ID NO: 69, amino acids 21-99 or amino acids 31-94 of SEQ ID NO: 70, amino acids 37-173 or amino acids 19-178 of SEQ ID NO: 71, or amino acids 37-199 of SEQ ID NO: 72.

In other aspects of this embodiment, an IL binding domain comprises a polypeptide having an amino acid identity of, e.g., at least 70%, at least 75%, at least 80%, at least 85%, at least 90%, at least 95%, or at least 97% to SEQ ID NO: 67, SEQ ID NO: 68, SEQ ID NO: 69, SEQ ID NO: 70, SEQ ID NO: 71, or SEQ ID NO: 72; or at most 70%, at most 75%, at most 80%, at most 85%, at most 90%, at most 95%, or at most 97% to SEQ ID NO: 67, SEQ ID NO: 68, SEQ ID NO: 69, SEQ ID NO: 70, SEQ ID NO: 71, or SEQ ID NO: 72. In yet other aspects of this embodiment, an IL binding domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 67, SEQ ID NO: 68, SEQ ID NO: 69, SEQ ID NO: 70, SEQ ID NO: 71, or SEQ ID NO: 72; or at most 1, 2, 3, 4, 5, 6, 7, 8, 9, 10, 15 or 20 non-contiguous amino acid deletions, additions, and/or substitutions relative to SEQ ID NO: 67, SEQ ID NO: 68, SEQ ID NO: 69, SEQ ID NO: 70, SEQ ID NO: 71, or SEQ ID NO: 72. In still other aspects of this embodiment, an IL binding domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 67, SEQ ID NO: 68, SEQ ID NO: 69, SEQ ID NO: 70, SEQ ID NO: 71, or SEQ ID NO: 72; or at most 1, 2, 3, 4, 5, 6, 7, 8, 9, 10, 15 or 20 contiguous amino acid deletions, additions, and/or substitutions relative to SEQ ID NO: 67, SEQ ID NO: 68, SEQ ID NO: 69, SEQ ID NO: 70, SEQ ID NO: 71, or SEQ ID NO: 72.

In other aspects of this embodiment, an IL binding domain comprises a polypeptide having an amino acid identity of, e.g., at least 70%, at least 75%, at least 80%, at least 85%, at least 90%, at least 95%, or at least 97% to amino acids 123-265 of SEQ ID NO: 67, amino acids 21-153 of SEQ ID NO: 68, amino acids 57-210 of SEQ ID NO: 69, amino acids 21-99 or amino acids 31-94 of SEQ ID NO: 70, amino acids 37-173 or amino acids 19-178 of SEQ ID NO: 71, or amino acids 37-199 of SEQ ID NO: 72; or at most 70%, at most 75%, at most 80%, at most 85%, at most 90%, at most 95%, or at most 97% to amino acids 123-265 of SEQ ID NO: 67, amino acids 21-153 of SEQ ID NO: 68, amino acids 57-210 of SEQ ID NO: 69, amino acids 21-99 or amino acids 31-94 of SEQ ID NO: 70, amino acids 37-173 or amino acids 19-178 of SEQ ID NO: 71, or amino acids 37-199 of SEQ ID NO: 72. In yet other aspects of this embodiment, an IL binding domain comprises a polypeptide having, e.g., at least 1, 2, 3, 4, 5, 6, 7, 8, 9, 10, 15 or 20 non-contiguous amino acid deletions, additions, and/or substitutions relative to amino acids 123-265 of SEQ ID NO: 67, amino acids 21-153 of SEQ ID NO: 68, amino acids 57-210 of SEQ ID NO: 69, amino acids 21-99 or amino acids 31-94 of SEQ ID NO: 70, amino acids 37-173 or amino acids 19-178 of SEQ ID NO: 71, or amino acids 37-199 of SEQ ID NO: 72; or at most 1, 2, 3, 4, 5, 6, 7, 8, 9, 10, 15 or 20 non-contiguous amino acid deletions, additions, and/or substitutions relative to amino acids 123-265 of SEQ ID NO: 67, amino acids 21-153 of SEQ ID NO: 68, amino acids 57-210 of SEQ ID NO: 69, amino acids 21-99 or amino acids 31-94 of SEQ ID NO: 70, amino acids 37-173 or amino acids 19-178 of SEQ ID NO: 71, or amino acids 37-199 of SEQ ID NO: 72. In still other aspects of this embodiment, an IL binding domain comprises a polypeptide having, e.g., at least 1, 2, 3, 4, 5, 6, 7, 8, 9, 10, 15 or 20 contiguous amino acid deletions, additions, and/or substitutions relative to amino acids 123-265 of SEQ ID NO: 67, amino acids 21-153 of SEQ ID NO: 68, amino acids 57-210 of SEQ ID NO: 69, amino acids 21-99 or amino acids 31-94 of SEQ ID NO: 70, amino acids 37-173 or amino acids 19-178 of SEQ ID NO: 71, or amino acids 37-199 of SEQ ID NO: 72; or at most 1, 2, 3, 4, 5, 6, 7, 8, 9, 10, 15 or 20 contiguous amino acid deletions, additions, and/or substitutions relative to amino acids 123-265 of SEQ ID NO: 67, amino acids 21-153 of SEQ ID NO: 68, amino acids 57-210 of SEQ ID NO: 69, amino acids 21-99 or amino acids 31-94 of SEQ ID NO: 70, amino acids 37-173 or amino acids 19-178 of SEQ ID NO: 71, or amino acids 37-199 of SEQ ID NO: 72.

Another example of a retargeted binding element disclosed in the present specification is a vascular endothelial growth factor (VEGF) peptide binding domain. Non-limiting examples of a VEGF peptide binding domain include a VEGF-A, a VEGF-B, a VEGF-C, a VEGF-D, or a placenta growth factor (PIGF).

Thus, in an embodiment, a retargeted binding element comprises a VEGF peptide binding domain. In aspects of this embodiment, a VEGF peptide binding domain comprises a VEGF-A, a VEGF-B, a VEGF-C, a VEGF-D, or a PIGF. In aspects of this embodiment, a VEGF peptide binding domain comprises SEQ ID NO: 73, SEQ ID NO: 74, SEQ ID NO: 75, SEQ ID NO: 76, SEQ ID NO: 77, or SEQ ID NO: 78. In other aspects of this embodiment, a VEGF peptide binding domain comprises amino acids 50-133 of SEQ ID NO: 73, amino acids 45-127 of SEQ ID NO: 74, amino acids 129-214 of SEQ ID NO: 75, amino acids 109-194 of SEQ ID NO: 76, amino acids 46-163, amino acids 49-162, amino acids 168-345, amino acids 244-306, or amino acids 248-340 of SEQ ID NO: 77, or amino acids 50-131 or amino acids 132-203 of SEQ ID NO: 78.

In other aspects of this embodiment, a VEGF peptide binding domain comprises a polypeptide having an amino acid identity of, e.g., at least 70%, at least 75%, at least 80%, at least 85%, at least 90%, at least 95%, or at least 97% to SEQ ID NO: 73, SEQ ID NO: 74, SEQ ID NO: 75, SEQ ID NO: 76, SEQ ID NO: 77, or SEQ ID NO: 78; or at most 70%, at most 75%, at most 80%, at most 85%, at most 90%, at most 95%, or at most 97% to SEQ ID NO: 73, SEQ ID NO: 74, SEQ ID NO: 75, SEQ ID NO: 76, SEQ ID NO: 77, or SEQ ID NO: 78. In yet other aspects of this embodiment, a VEGF peptide binding domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 73, SEQ ID NO: 74, SEQ ID NO: 75, SEQ ID NO: 76, SEQ ID NO: 77, or SEQ ID NO: 78; or at most 1, 2, 3, 4, 5, 6, 7, 8, 9, 10, 15 or 20 non-contiguous amino acid deletions, additions, and/or substitutions relative to SEQ ID NO: 73, SEQ ID NO: 74, SEQ ID NO: 75, SEQ ID NO: 76, SEQ ID NO: 77, or SEQ ID NO: 78. In still other aspects of this embodiment, a VEGF peptide binding domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 73, SEQ ID NO: 74, SEQ ID NO: 75, SEQ ID NO: 76, SEQ ID NO: 77, or SEQ ID NO: 78; or at most 1, 2, 3, 4, 5, 6, 7, 8, 9, 10, 15 or 20 contiguous amino acid deletions, additions, and/or substitutions relative to SEQ ID NO: 73, SEQ ID NO: 74, SEQ ID NO: 75, SEQ ID NO: 76, SEQ ID NO: 77, or SEQ ID NO: 78.

In other aspects of this embodiment, a VEGF peptide binding domain comprises a polypeptide having an amino acid identity of, e.g., at least 70%, at least 75%, at least 80%, at least 85%, at least 90%, at least 95%, or at least 97% to amino acids 50-133 of SEQ ID NO: 73, amino acids 45-127 of SEQ ID NO: 74, amino acids 129-214 of SEQ ID NO: 75, amino acids 109-194 of SEQ ID NO: 76, amino acids 46-163, amino acids 49-162, amino acids 168-345, amino acids 244-306, or amino acids 248-340 of SEQ ID NO: 77, or amino acids 50-131 or amino acids 132-203 of SEQ ID NO: 78; or at most 70%, at most 75%, at most 80%, at most 85%, at most 90%, at most 95%, or at most 97% to amino acids 50-133 of SEQ ID NO: 73, amino acids 45-127 of SEQ ID NO: 74, amino acids 129-214 of SEQ ID NO: 75, amino acids 109-194 of SEQ ID NO: 76, amino acids 46-163, amino acids 49-162, amino acids 168-345, amino acids 244-306, or amino acids 248-340 of SEQ ID NO: 77, or amino acids 50-131 or amino acids 132-203 of SEQ ID NO: 78. In yet other aspects of this embodiment, a VEGF peptide binding domain comprises a polypeptide having, e.g., at least 1, 2, 3, 4, 5, 6, 7, 8, 9, 10, 15 or 20 non-contiguous amino acid deletions, additions, and/or substitutions relative to amino acids 50-133 of SEQ ID NO: 73, amino acids 45-127 of SEQ ID NO: 74, amino acids 129-214 of SEQ ID NO: 75, amino acids 109-194 of SEQ ID NO: 76, amino acids 46-163, amino acids 49-162, amino acids 168-345, amino acids 244-306, or amino acids 248-340 of SEQ ID NO: 77, or amino acids 50-131 or amino acids 132-203 of SEQ ID NO: 78; or at most 1, 2, 3, 4, 5, 6, 7, 8, 9, 10, 15 or 20 non-contiguous amino acid deletions, additions, and/or substitutions relative to amino acids 50-133 of SEQ ID NO: 73, amino acids 45-127 of SEQ ID NO: 74, amino acids 129-214 of SEQ ID NO: 75, amino acids 109-194 of SEQ ID NO: 76, amino acids 46-163, amino acids 49-162, amino acids 168-345, amino acids 244-306, or amino acids 248-340 of SEQ ID NO: 77, or amino acids 50-131 or amino acids 132-203 of SEQ ID NO: 78. In still other aspects of this embodiment, a VEGF peptide binding domain comprises a polypeptide having, e.g., at least 1, 2, 3, 4, 5, 6, 7, 8, 9, 10, 15 or 20 contiguous amino acid deletions, additions, and/or substitutions relative to amino acids 50-133 of SEQ ID NO: 73, amino acids 45-127 of SEQ ID NO: 74, amino acids 129-214 of SEQ ID NO: 75, amino acids 109-194 of SEQ ID NO: 76, amino acids 46-163, amino acids 49-162, amino acids 168-345, amino acids 244-306, or amino acids 248-340 of SEQ ID NO: 77, or amino acids 50-131 or amino acids 132-203 of SEQ ID NO: 78; or at most 1, 2, 3, 4, 5, 6, 7, 8, 9, 10, 15 or 20 contiguous amino acid deletions, additions, and/or substitutions relative to amino acids 50-133 of SEQ ID NO: 73, amino acids 45-127 of SEQ ID NO: 74, amino acids 129-214 of SEQ ID NO: 75, amino acids 109-194 of SEQ ID NO: 76, amino acids 46-163, amino acids 49-162, amino acids 168-345, amino acids 244-306, or amino acids 248-340 of SEQ ID NO: 77, or amino acids 50-131 or amino acids 132-203 of SEQ ID NO: 78.

Another example of a retargeted binding element disclosed in the present specification is an insulin-like growth factor (IGF) peptide binding domain. Non-limiting examples of an IGF peptide binding domain include an IGF-1 or an IGF-2.

Thus, in an embodiment, a retargeted binding element comprises an IGF peptide binding domain. In aspects of this embodiment, an IGF peptide binding domain comprises an IGF-1 or an IGF-2. In aspects of this embodiment, an IGF peptide binding domain comprises SEQ ID NO: 79 or SEQ ID NO: 80. In other aspects of this embodiment, an IGF peptide binding domain comprises amino acids 52-109 or amino acids 49-118 of SEQ ID NO: 79, or amino acids 31-84 or amino acids 25-180 of SEQ ID NO: 80.

In other aspects of this embodiment, an IGF peptide binding domain comprises a polypeptide having an amino acid identity of, e.g., at least 70%, at least 75%, at least 80%, at least 85%, at least 90%, at least 95%, or at least 97% to SEQ ID NO: 79 or SEQ ID NO: 80; or at most 70%, at most 75%, at most 80%, at most 85%, at most 90%, at most 95%, or at most 97% to SEQ ID NO: 79 or SEQ ID NO: 80. In yet other aspects of this embodiment, an IGF peptide binding domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 79 or SEQ ID NO: 80; or at most 1, 2, 3, 4, 5, 6, 7, 8, 9, 10, or 20 non-contiguous amino acid deletions, additions, and/or substitutions relative to SEQ ID NO: 79 or SEQ ID NO: 80. In still other aspects of this embodiment, an IGF peptide binding domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 79 or SEQ ID NO: 80; or at most 1, 2, 3, 4, 5, 6, 7, 8, 9, 10, 15 or 20 contiguous amino acid deletions, additions, and/or substitutions relative to SEQ ID NO: 79 or SEQ ID NO: 80.

In other aspects of this embodiment, an IGF peptide binding domain comprises a polypeptide having an amino acid identity of, e.g., at least 70%, at least 75%, at least 80%, at least 85%, at least 90%, at least 95%, or at least 97% to amino acids 52-109 or amino acids 49-118 of SEQ ID NO: 79, or amino acids 31-84 or amino acids 25-180 of SEQ ID NO: 80; or at most 70%, at most 75%, at most 80%, at most 85%, at most 90%, at most 95%, or at most 97% to amino acids 52-109 or amino acids 49-118 of SEQ ID NO: 79, or amino acids 31-84 or amino acids 25-180 of SEQ ID NO: 80. In yet other aspects of this embodiment, an IGF peptide binding domain comprises a polypeptide having, e.g., at least 1, 2, 3, 4, 5, 6, 7, 8, 9, 10, 15 or 20 non-contiguous amino acid deletions, additions, and/or substitutions relative to amino acids 52-109 or amino acids 49-118 of SEQ ID NO: 79, or amino acids 31-84 or amino acids 25-180 of SEQ ID NO: 80; or at most 1, 2, 3, 4, 5, 6, 7, 8, 9, 10, 15 or 20 non-contiguous amino acid deletions, additions, and/or substitutions relative to amino acids 52-109 or amino acids 49-118 of SEQ ID NO: 79, or amino acids 31-84 or amino acids 25-180 of SEQ ID NO: 80. In still other aspects of this embodiment, an IGF peptide binding domain comprises a polypeptide having, e.g., at least 1, 2, 3, 4, 5, 6, 7, 8, 9, 10, 15 or 20 contiguous amino acid deletions, additions, and/or substitutions relative to amino acids 52-109 or amino acids 49-118 of SEQ ID NO: 79, or amino acids 31-84 or amino acids 25-180 of SEQ ID NO: 80; or at most 1, 2, 3, 4, 5, 6, 7, 8, 9, 10, 15 or 20 contiguous amino acid deletions, additions, and/or substitutions relative to amino acids 52-109 or amino acids 49-118 of SEQ ID NO: 79, or amino acids 31-84 or amino acids 25-180 of SEQ ID NO: 80.

Another example of a retargeted binding element disclosed in the present specification is an epidermal growth factor (EGF) peptide binding domain. Non-limiting examples of an EGF peptide binding domain include an EGF, a heparin-binding EGF-like growth factor (HB-EGF), a transforming growth factor-α (TGF-α), an amphiregulin (AR), an epiregulin (EPR), an epigen (EPG), a betacellulin (BTC), a neuregulin-1 (NRG1), a neuregulin-2 (NRG2), a neuregulin-3, (NRG3), or a neuregulin-4 (NRG4).

Thus, in an embodiment, a retargeted binding element comprises an EGF peptide binding domain. In aspects of this embodiment, an EGF peptide binding domain comprises an EGF, a HB-EGF, a TGF-α, an AR, an EPR, an EPG, a BTC, a NRG-1, a NRG-2, a NRG-3, or a NRG-4. In aspects of this embodiment, an EGF peptide binding domain comprises SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91. In other aspects of this embodiment, an EGF peptide binding domain comprises amino acids 101-251 or amino acids 107-251 of SEQ ID NO: 84, amino acids 63-108 of SEQ ID NO: 85, amino acids 23-154 of SEQ ID NO: 86, amino acids 235-630 of SEQ ID NO: 88, amino acids 398-718 of SEQ ID NO: 89, or amino acids 353-648 of SEQ ID NO: 90. In yet another aspect of this embodiment, an EGF peptide binding domain comprises a NRG-2 isoform like a NRG-2 isoform 1, a NRG-2 isoform 2, a NRG-2 isoform 3, a NRG-2 isoform 4, a NRG-2 isoform 5, or a NRG-2 isoform 6.

In other aspects of this embodiment, an EGF peptide binding domain comprises a polypeptide having an amino acid identity of, e.g., at least 70%, at least 75%, at least 80%, at least 85%, at least 90%, at least 95%, or at least 97% to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91; or at most 70%, at most 75%, at most 80%, at most 85%, at most 90%, at most 95%, or at most 97% to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91. In yet other aspects of this embodiment, an EGF peptide binding domain comprises a polypeptide having, e.g., at least 1, 2, 3, 4, 5, 6, 7, 8, 9, 10, 15 or 20 non-contiguous amino acid deletions, additions, and/or substitutions relative to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91; or at most 1, 2, 3, 4, 5, 6, 7, 8, 9, 10, 15 or 20 non-contiguous amino acid deletions, additions, and/or substitutions relative to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91. In still other aspects of this embodiment, an EGF peptide binding domain comprises a polypeptide having, e.g., at least 1, 2, 3, 4, 5, 6, 7, 8, 9, 10, 15 or 20 contiguous amino acid deletions, additions, and/or substitutions relative to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91; or at most 1, 2, 3, 4, 5, 6, 7, 8, 9, 10, 15 or 20 contiguous amino acid deletions, additions, and/or substitutions relative to SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91.

In other aspects of this embodiment, an EGF peptide binding domain comprises a polypeptide having an amino acid identity of, e.g., at least 70%, at least 75%, at least 80%, at least 85%, at least 90%, at least 95%, or at least 97% to amino acids 101-251 or amino acids 107-251 of SEQ ID NO: 84, amino acids 63-108 of SEQ ID NO: 85, amino acids 23-154 of SEQ ID NO: 86, amino acids 235-630 of SEQ ID NO: 88, amino acids 398-718 of SEQ ID NO: 89, or amino acids 353-648 of SEQ ID NO: 90; or at most 70%, at most 75%, at most 80%, at most 85%, at most 90%, at most 95%, or at most 97% to amino acids 101-251 or amino acids 107-251 of SEQ ID NO: 84, amino acids 63-108 of SEQ ID NO: 85, amino acids 23-154 of SEQ ID NO: 86, amino acids 235-630 of SEQ ID NO: 88, amino acids 398-718 of SEQ ID NO: 89, or amino acids 353-648 of SEQ ID NO: 90. In yet other aspects of this embodiment, an EGF peptide binding domain comprises a polypeptide having, e.g., at least 1, 2, 3, 4, 5, 6, 7, 8, 9, 10, 15 or 20 non-contiguous amino acid deletions, additions, and/or substitutions relative to amino acids 101-251 or amino acids 107-251 of SEQ ID NO: 84, amino acids 63-108 of SEQ ID NO: 85, amino acids 23-154 of SEQ ID NO: 86, amino acids 235-630 of SEQ ID NO: 88, amino acids 398-718 of SEQ ID NO: 89, or amino acids 353-648 of SEQ ID NO: 90; or at most 1, 2, 3, 4, 5, 6, 7, 8, 9, 10, 15 or 20 non-contiguous amino acid deletions, additions, and/or substitutions relative to amino acids 101-251 or amino acids 107-251 of SEQ ID NO: 84, amino acids 63-108 of SEQ ID NO: 85, amino acids 23-154 of SEQ ID NO: 86, amino acids 235-630 of SEQ ID NO: 88, amino acids 398-718 of SEQ ID NO: 89, or amino acids 353-648 of SEQ ID NO: 90. In still other aspects of this embodiment, an EGF peptide binding domain comprises a polypeptide having, e.g., at least 1, 2, 3, 4, 5, 6, 7, 8, 9, 10, 15 or 20 contiguous amino acid deletions, additions, and/or substitutions relative to amino acids 101-251 or amino acids 107-251 of SEQ ID NO: 84, amino acids 63-108 of SEQ ID NO: 85, amino acids 23-154 of SEQ ID NO: 86, amino acids 235-630 of SEQ ID NO: 88, amino acids 398-718 of SEQ ID NO: 89, or amino acids 353-648 of SEQ ID NO: 90; or at most 1, 2, 3, 4, 5, 6, 7, 8, 9, 10, 15 or 20 contiguous amino acid deletions, additions, and/or substitutions relative to amino acids 101-251 or amino acids 107-251 of SEQ ID NO: 84, amino acids 63-108 of SEQ ID NO: 85, amino acids 23-154 of SEQ ID NO: 86, amino acids 235-630 of SEQ ID NO: 88, amino acids 398-718 of SEQ ID NO: 89, or amino acids 353-648 of SEQ ID NO: 90.

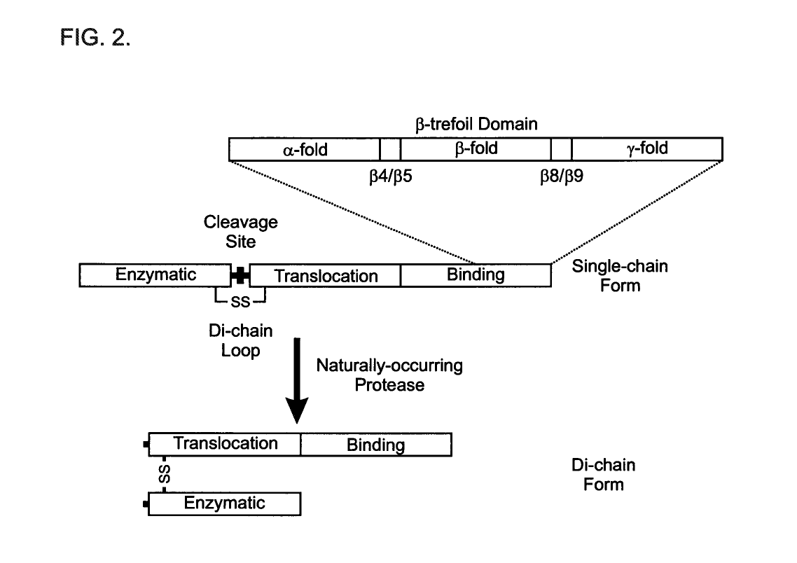

Clostridial toxins are each translated as a single-chain polypeptide of approximately 150 kDa that is subsequently cleaved by proteolytic scission within a disulfide loop by a naturally-occurring protease. This cleavage occurs within the discrete di-chain loop region created between two cysteine residues that form a disulfide bridge. This posttranslational processing yields a di-chain molecule comprising an approximately 50 kDa light chain (LC) and an approximately 100 kDa heavy chain (HC) held together by the single disulfide bond and non-covalent interactions between the two chains (FIG. 2). To facilitate recombinant production of a TVEMP, an exogenous protease cleavage site can be used to convert the single-chain polypeptide form of a TVEMP disclosed in the present specification into the di-chain form. See, e.g., Steward, L. E. et al., Modified Clostridial Toxins with Enhanced Targeting Capabilities For Endogenous Clostridial Toxin Receptor Systems, U.S. Patent Publication No. US 2008/0096248 (Apr. 24, 2008); Steward, L. E. et al., Activatable Clostridial Toxins, U.S. Patent Publication No. US 2008/0032930 (Feb. 7, 2008); Steward, supra, (2007); Dolly, supra, (2007); Foster, supra, WO 2006/059093 (2006); and Foster, supra, WO 2006/059105 (2006), each of which is hereby incorporated by reference in its entirety.

It is envisioned that any and all protease cleavage sites can be used to convert the single-chain polypeptide form of a Clostridial toxin into the di-chain form, including, without limitation, endogenous di-chain loop protease cleavage sites and exogenous protease cleavage sites. Thus, in an aspect of the invention, a TVEMP comprises, in part, an endogenous protease cleavage site within a di-chain loop region. In another aspect of the invention, a TVEMP comprises, in part, an exogenous protease cleavage site within a di-chain loop region. As used herein, the term “di-chain loop region” refers to the amino acid sequence of a Clostridial toxin containing a protease cleavage site used to convert the single-chain form of a Clostridial toxin into the di-chain form. Non-limiting examples of a Clostridial toxin di-chain loop region, include, a di-chain loop region of BoNT/A comprising amino acids 430-454 of SEQ ID NO: 1; a di-chain loop region of BoNT/B comprising amino acids 437-446 of SEQ ID NO: 2; a di-chain loop region of BoNT/C1 comprising amino acids 437-453 of SEQ ID NO: 3; a di-chain loop region of BoNT/D comprising amino acids 437-450 of SEQ ID NO: 4; a di-chain loop region of BoNT/E comprising amino acids 412-426 of SEQ ID NO: 5; a di-chain loop region of BoNT/F comprising amino acids 429-445 of SEQ ID NO: 6; a di-chain loop region of BoNT/G comprising amino acids 436-450 of SEQ ID NO: 7; and a di-chain loop region of TeNT comprising amino acids 439-467 of SEQ ID NO: 8 (Table 4).

TABLE 4
Di-chain Loop Region

| | Di-chain Loop Region Containing |
|---|---|
| | the Naturally-occurring |
| Toxin | Protease Cleavage Site |
| BoNT/A | CVRGIITSKTKSLDKGYNK*----ALNDLC |
| BoNT/B | CKSVK*-------------------APGIC |
| BoNT/C1 | CHKAIDGRSLYNK*------------TLDC |
| BoNT/D | CLRLTKNSR*---------------DDSTC |
| BoNT/E | CKNIVSVKGIR*--------------KSIC |
| BoNT/F | CKSVIPRKGTK*------------APPRLC |
| BoNT/G | CKPVMYKNTGK*--------------SEQC |
| TeNT | CKKIIPPTNIRENLYNRTA*SLTDLGGELC |
| BaNT | CKS-IVSKKGTK*------------NSLC |
| BuNT | CKN-IVSVKGIR*--------------KSIC |

The amino acid sequence displayed are as follows:
BoNT/A, residues 430-454 of SEQ ID NO: 1;
BoNT/B, residues 437-446 of SEQ ID NO: 2;
BoNT/C1, residues 437-453 of SEQ ID NO: 3;
BoNT/D, residues 437-450 of SEQ ID NO: 4;
BoNT/E, residues 412-426 of SEQ ID NO: 5;
BoNT/F, residues 429-445 of SEQ ID NO: 6;
BoNT/G, residues 436-450 of SEQ ID NO: 7;
TeNT, residues 439-467 of SEQ ID NO: 8;
BaNT, residues 421-435 of SEQ ID NO: 9; and
BuNT, residues 412-426 of SEQ ID NO: 10.
An asterisks (*) indicates the peptide bond that is cleaved by a Clostridial toxin protease.

As used herein, the term “endogenous di-chain loop protease cleavage site” is synonymous with a “naturally occurring di-chain loop protease cleavage site” and refers to a naturally occurring protease cleavage site found within the di-chain loop region of a naturally occurring Clostridial toxin and includes, without limitation, naturally occurring Clostridial toxin di-chain loop protease cleavage site variants, such as, e.g., Clostridial toxin di-chain loop protease cleavage site isoforms and Clostridial toxin di-chain loop protease cleavage site subtypes. Non-limiting examples of an endogenous protease cleavage site, include, e.g., a BoNT/A di-chain loop protease cleavage site, a BoNT/B di-chain loop protease cleavage site, a BoNT/C1 di-chain loop protease cleavage site, a BoNT/D di-chain loop protease cleavage site, a BoNT/E di-chain loop protease cleavage site, a BoNT/F di-chain loop protease cleavage site, a BoNT/G di-chain loop protease cleavage site and a TeNT di-chain loop protease cleavage site.

As mentioned above, Clostridial toxins are translated as a single-chain polypeptide of approximately 150 kDa that is subsequently cleaved by proteolytic scission within a disulfide loop by a naturally-occurring protease. This posttranslational processing yields a di-chain molecule comprising an approximately 50 kDa light chain (LC) and an approximately 100 kDa heavy chain (HC) held together by a single disulphide bond and noncovalent interactions. While the identity of the protease is currently unknown, the di-chain loop protease cleavage site for many Clostridial toxins has been determined. In BoNTs, cleavage at K448-A449 converts the single polypeptide form of BoNT/A into the di-chain form; cleavage at K441-A442 converts the single polypeptide form of BoNT/B into the di-chain form; cleavage at K449-T450 converts the single polypeptide form of BoNT/C1 into the di-chain form; cleavage at R445-D446 converts the single polypeptide form of BoNT/D into the di-chain form; cleavage at R422-K423 converts the single polypeptide form of BoNT/E into the di-chain form; cleavage at K439-A440 converts the single polypeptide form of BoNT/F into the di-chain form; and cleavage at K446-S447 converts the single polypeptide form of BoNT/G into the di-chain form. Proteolytic cleavage of the single polypeptide form of TeNT at A457-S458 results in the di-chain form. Proteolytic cleavage of the single polypeptide form of BaNT at K431-N432 results in the di-chain form. Proteolytic cleavage of the single polypeptide form of BuNT at R422-K423 results in the di-chain form. Such a di-chain loop protease cleavage site is operably-linked in-frame to a TVEMP as a fusion protein. However, it should also be noted that additional cleavage sites within the di-chain loop also appear to be cleaved resulting in the generation of a small peptide fragment being lost. As a non-limiting example, BoNT/A single-chain polypeptide cleavage ultimately results in the loss of a ten amino acid fragment within the di-chain loop.

Thus, in an embodiment, a protease cleavage site comprising an endogenous Clostridial toxin di-chain loop protease cleavage site is used to convert the single-chain toxin into the di-chain form. In aspects of this embodiment, conversion into the di-chain form by proteolytic cleavage occurs from a site comprising, e.g., a BoNT/A di-chain loop protease cleavage site, a BoNT/B di-chain loop protease cleavage site, a BoNT/C1 di-chain loop protease cleavage site, a BoNT/D di-chain loop protease cleavage site, a BoNT/E di-chain loop protease cleavage site, a BoNT/F di-chain loop protease cleavage site, a BoNT/G di-chain loop protease cleavage site, a TeNT di-chain loop protease cleavage site, a BaNT di-chain loop protease cleavage site, or a BuNT di-chain loop protease cleavage site.

In other aspects of this embodiment, conversion into the di-chain form by proteolytic cleavage occurs from a site comprising, e.g., a di-chain loop region of BoNT/A comprising amino acids 430-454 of SEQ ID NO: 1; a di-chain loop region of BoNT/B comprising amino acids 437-446 of SEQ ID NO: 2; a di-chain loop region of BoNT/C1 comprising amino acids 437-453 of SEQ ID NO: 3; a di-chain loop region of BoNT/D comprising amino acids 437-450 of SEQ ID NO: 4; a di-chain loop region of BoNT/E comprising amino acids 412-426 of SEQ ID NO: 5; a di-chain loop region of BoNT/F comprising amino acids 429-445 of SEQ ID NO: 6; a di-chain loop region of BoNT/G comprising amino acids 436-450 of SEQ ID NO: 7; or a di-chain loop region of TeNT comprising amino acids 439-467 of SEQ ID NO: 8; a di-chain loop region of BaNT comprising amino acids 421-435 of SEQ ID NO: 9; or a di-chain loop region of BuNT comprising amino acids 412-426 of SEQ ID NO: 10.

It is also envisioned that an exogenous protease cleavage site can be used to convert the single-chain polypeptide form of a TVEMP disclosed in the present specification into the di-chain form. As used herein, the term “exogenous protease cleavage site” is synonymous with a “non-naturally occurring protease cleavage site” or “non-native protease cleavage site” and refers to a protease cleavage site that is not normally present in a di-chain loop region from a naturally occurring Clostridial toxin, with the proviso that the exogenous protease cleavage site is not a human protease cleavage site or a protease cleavage site that is susceptible to a protease being expressed in the host cell that is expressing a construct encoding an activatable polypeptide disclosed in the present specification. It is envisioned that any and all exogenous protease cleavage sites can be used to convert the single-chain polypeptide form of a Clostridial toxin into the di-chain form are useful to practice aspects of the present invention. Non-limiting examples of exogenous protease cleavage sites include, e.g., a plant papain cleavage site, an insect papain cleavage site, a crustacian papain cleavage site, an enterokinase cleavage site, a human rhinovirus 3C protease cleavage site, a human enterovirus 3C protease cleavage site, a tobacco etch virus (TEV) protease cleavage site, a Tobacco Vein Mottling Virus (TVMV) cleavage site, a subtilisin cleavage site, a hydroxylamine cleavage site, or a Caspase 3 cleavage site.

It is envisioned that an exogenous protease cleavage site of any and all lengths can be useful in aspects of the present invention with the proviso that the exogenous protease cleavage site is capable of being cleaved by its respective protease. Thus, in aspects of this embodiment, an exogenous protease cleavage site can have a length of, e.g., at least 6, 7, 8, 9, 10, 15, 20, 25, 30, 40, 50, or at least 60 amino acids; or at most 6, 7, 8, 9, 10, 15, 20, 25, 30, 40, 50, or at least 60 amino acids.

In an embodiment, an exogenous protease cleavage site is located within the di-chain loop of a TVEMP. In aspects of this embodiment, a TVEMP comprises an exogenous protease cleavage site comprises, e.g., a plant papain cleavage site, an insect papain cleavage site, a crustacian papain cleavage site, a non-human enterokinase protease cleavage site, a Tobacco Etch Virus protease cleavage site, a Tobacco Vein Mottling Virus protease cleavage site, a human rhinovirus 3C protease cleavage site, a human enterovirus 3C protease cleavage site, a subtilisin cleavage site, a hydroxylamine cleavage site, a SUMO/ULP-1 protease cleavage site, and a non-human Caspase 3 cleavage site. In other aspects of this embodiment, an exogenous protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In an aspect of this embodiment, an exogenous protease cleavage site can comprise, e.g., a non-human enterokinase cleavage site is located within the di-chain loop of a TVEMP. In other aspects of the embodiment, an exogenous protease cleavage site can comprise, e.g., a bovine enterokinase protease cleavage site located within the di-chain loop of a TVEMP. In other aspects of the embodiment, an exogenous protease cleavage site can comprise, e.g., a bovine enterokinase protease cleavage site located within the di-chain loop of a TVEMP comprises SEQ ID NO: 21. In still other aspects of this embodiment, a bovine enterokinase protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In another aspect of this embodiment, an exogenous protease cleavage site can comprise, e.g., a Tobacco Etch Virus protease cleavage site is located within the di-chain loop of a TVEMP. In other aspects of the embodiment, an exogenous protease cleavage site can comprise, e.g., a Tobacco Etch Virus protease cleavage site located within the di-chain loop of a TVEMP comprises the consensus sequence E-P5-P4-Y-P2-Q*-G (SEQ ID NO: 22) or E-P5-P4-Y-P2-Q*-S (SEQ ID NO: 23), where P2, P4 and P5 can be any amino acid. In other aspects of the embodiment, an exogenous protease cleavage site can comprise, e.g., a Tobacco Etch Virus protease cleavage site located within the di-chain loop of a TVEMP comprises SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32 or SEQ ID NO: 33. In still other aspects of this embodiment, a Tobacco Etch Virus protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In another aspect of this embodiment, an exogenous protease cleavage site can comprise, e.g., a Tobacco Vein Mottling Virus protease cleavage site is located within the di-chain loop of a TVEMP. In other aspects of the embodiment, an exogenous protease cleavage site can comprise, e.g., a Tobacco Vein Mottling Virus protease cleavage site located within the di-chain loop of a TVEMP comprises the consensus sequence P6-P5-V-R-F-Q*-G (SEQ ID NO: 34) or P6-P5-V-R-F-Q*-S (SEQ ID NO: 35), where P5 and P6 can be any amino acid. In other aspects of the embodiment, an exogenous protease cleavage site can comprise, e.g., a Tobacco Vein Mottling Virus protease cleavage site located within the di-chain loop of a TVEMP comprises SEQ ID NO: 36, SEQ ID NO: 37, SEQ ID NO: 38, or SEQ ID NO: 39. In still other aspects of this embodiment, a Tobacco Vein Mottling Virus protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In still another aspect of this embodiment, an exogenous protease cleavage site can comprise, e.g., a human rhinovirus 3C protease cleavage site is located within the di-chain loop of a TVEMP. In other aspects of the embodiment, an exogenous protease cleavage site can comprise, e.g., a human rhinovirus 3C protease cleavage site located within the di-chain loop of a TVEMP comprises the consensus sequence P5-P4-L-F-Q*-G-P (SEQ ID NO: 40), where P4 is G, A, V, L, I, M, S or T and P5 can any amino acid, with D or E preferred. In other aspects of the embodiment, an exogenous protease cleavage site can comprise, e.g., a human rhinovirus 3C protease cleavage site located within the di-chain loop of a TVEMP comprises SEQ ID NO: 41, SEQ ID NO: 42, SEQ ID NO: 43, SEQ ID NO: 44, SEQ ID NO: 45 or SEQ ID NO: 46. In other aspects of the embodiment, an exogenous protease cleavage site can comprise, e.g., a human rhinovirus 3C protease located within the di-chain loop of a TVEMP that can be cleaved by PRESCISSION®, a modified human rhinovirus 3C protease (GE Healthcare Biosciences, Piscataway, N.J.). In still other aspects of this embodiment, a human rhinovirus 3C protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In yet another aspect of this embodiment, an exogenous protease cleavage site can comprise, e.g., a subtilisin cleavage site is located within the di-chain loop of a TVEMP. In other aspects of the embodiment, an exogenous protease cleavage site can comprise, e.g., a subtilisin cleavage site located within the di-chain loop of a TVEMP comprises the consensus sequence P6-P5-P4-P3-H*-Y (SEQ ID NO: 47) or P6-P5-P4-P3-Y-H* (SEQ ID NO: 48), where P3, P4 and P5 and P6 can be any amino acid. In other aspects of the embodiment, an exogenous protease cleavage site can comprise, e.g., a subtilisin cleavage site located within the di-chain loop of a TVEMP comprises SEQ ID NO: 49, SEQ ID NO: 50, or SEQ ID NO: 51. In other aspects of the embodiment, an exogenous protease cleavage site can comprise, e.g., a subtilisin cleavage site located within the di-chain loop of a TVEMP that can be cleaved by GENENASE®, a modified subtilisin (New England Biolabs, Ipswich, Mass.). In still other aspects of this embodiment, a subtilisin cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In yet another aspect of this embodiment, an exogenous protease cleavage site can comprise, e.g., a hydroxylamine cleavage site is located within the di-chain loop of a TVEMP. In other aspects of the embodiment, an exogenous protease cleavage site can comprise, e.g., a hydroxylamine cleavage site comprising multiples of the dipeptide NSG. In other aspects of the embodiment, an exogenous protease cleavage site can comprise, e.g., a hydroxylamine cleavage site located within the di-chain loop of a TVEMP comprises SEQ ID NO: 52, or SEQ ID NO: 53. In still other aspects of this embodiment, a hydroxylamine cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In yet another aspect of this embodiment, an exogenous protease cleavage site can comprise, e.g., a SUMO/ULP-1 protease cleavage site is located within the di-chain loop of a TVEMP. In other aspects of the embodiment, an exogenous protease cleavage site can comprise, e.g., a SUMO/ULP-1 protease cleavage site located within the di-chain loop of a TVEMP comprising the consensus sequence G-G*-P1′-P2′-P3′ (SEQ ID NO: 54), where P1′, P2′, and P3′ can be any amino acid. In other aspects of the embodiment, an exogenous protease cleavage site can comprise, e.g., a SUMO/ULP-1 protease cleavage site located within the di-chain loop of a TVEMP comprises SEQ ID NO: 55. In still other aspects of this embodiment, a SUMO/ULP-1 protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

In an aspect of this embodiment, an exogenous protease cleavage site can comprise, e.g., a non-human Caspase 3 cleavage site is located within the di-chain loop of a TVEMP. In other aspects of the embodiment, an exogenous protease cleavage site can comprise, e.g., a mouse Caspase 3 protease cleavage site located within the di-chain loop of a TVEMP. In other aspects of the embodiment, an exogenous protease cleavage site can comprise, e.g., a non-human Caspase 3 protease cleavage site located within the di-chain loop of a TVEMP comprises the consensus sequence D-P3-P2-D*P1′ (SEQ ID NO: 56), where P3 can be any amino acid, with E preferred, P2 can be any amino acid and P1′ can any amino acid, with G or S preferred. In other aspects of the embodiment, an exogenous protease cleavage site can comprise, e.g., a non-human Caspase 3 protease cleavage site located within the di-chain loop of a TVEMP comprising SEQ ID NO: 57, SEQ ID NO: 58, SEQ ID NO: 59, SEQ ID NO: 60, SEQ ID NO: 61, or SEQ ID NO: 62. In still other aspects of this embodiment, a bovine enterokinase protease cleavage site is located within the di-chain loop of, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

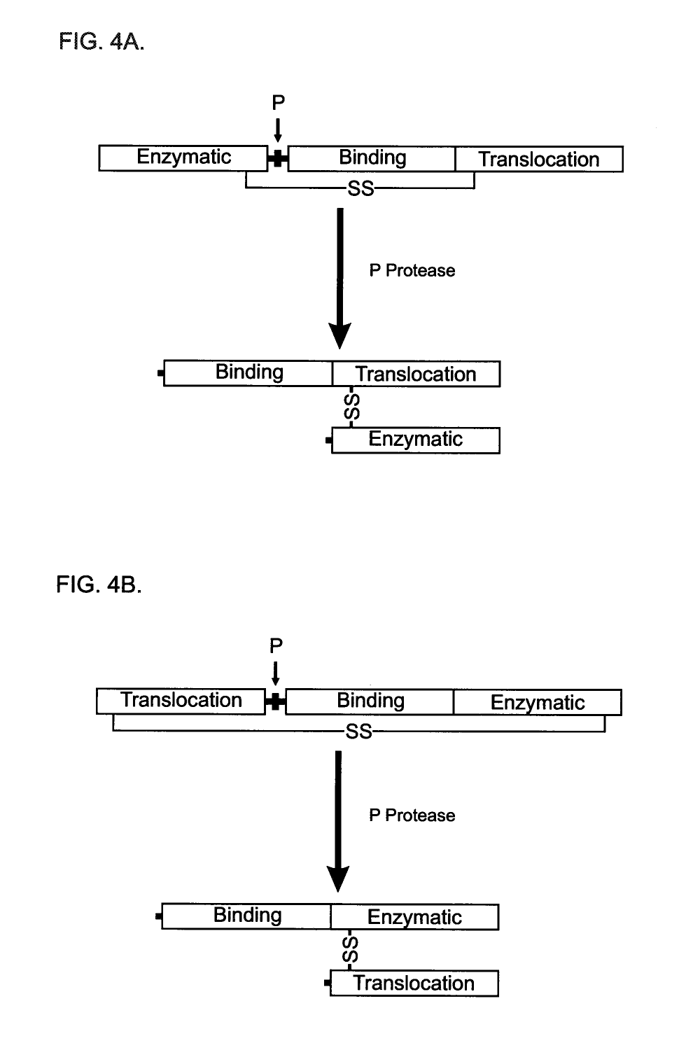

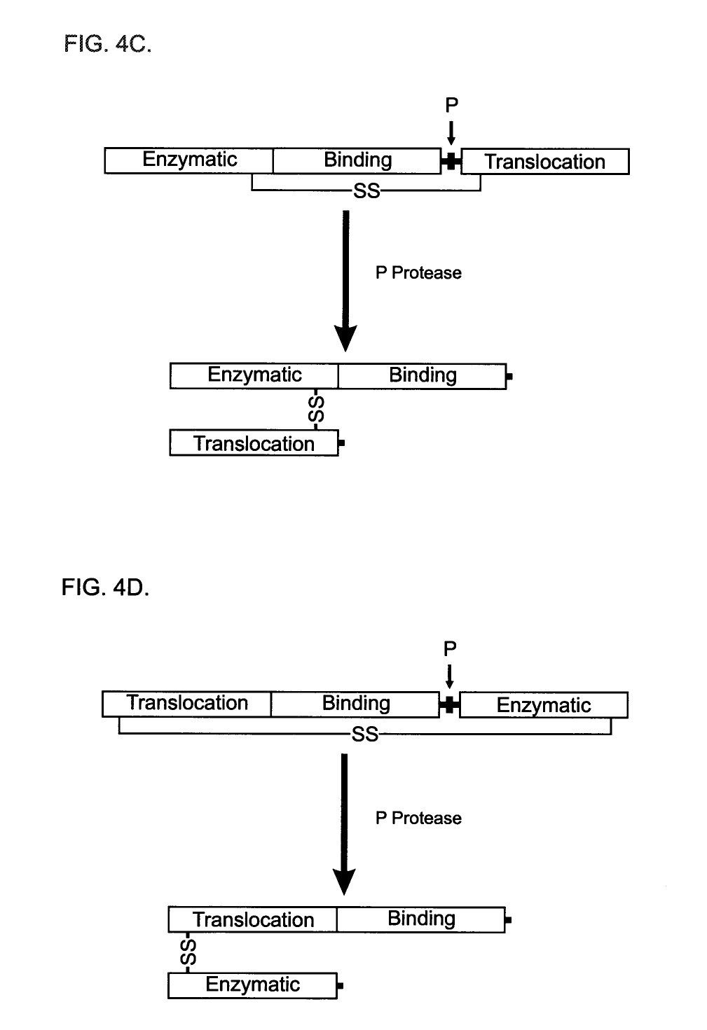

A di-chain loop region is modified to replace a naturally-occurring di-chain loop protease cleavage site for an exogenous protease cleavage site. In this modification, the naturally-occurring di-chain loop protease cleavage site is made inoperable and thus can not be cleaved by its protease. Only the exogenous protease cleavage site can be cleaved by its corresponding exogenous protease. In this type of modification, the exogenous protease site is operably-linked in-frame to a TVEMP as a fusion protein and the site can be cleaved by its respective exogenous protease. Replacement of an endogenous di-chain loop protease cleavage site with an exogenous protease cleavage site can be a substitution of the sites where the exogenous site is engineered at the position approximating the cleavage site location of the endogenous site. Replacement of an endogenous di-chain loop protease cleavage site with an exogenous protease cleavage site can be an addition of an exogenous site where the exogenous site is engineered at the position different from the cleavage site location of the endogenous site, the endogenous site being engineered to be inoperable. The location and kind of protease cleavage site may be critical because certain binding domains require a free amino-terminal or carboxyl-terminal amino acid. For example, when a retargeted peptide binding domain is placed between two other domains, e.g., see FIG. 4, a criterion for selection of a protease cleavage site could be whether the protease that cleaves its site leaves a flush cut, exposing the free amino-terminal or carboxyl-terminal of the binding domain necessary for selective binding of the binding domain to its receptor.

A naturally-occurring protease cleavage site can be made inoperable by altering at least the two amino acids flanking the peptide bond cleaved by the naturally-occurring di-chain loop protease. More extensive alterations can be made, with the proviso that the two cysteine residues of the di-chain loop region remain intact and the region can still form the disulfide bridge. Non-limiting examples of an amino acid alteration include deletion of an amino acid or replacement of the original amino acid with a different amino acid. Thus, in one embodiment, a naturally-occurring protease cleavage site is made inoperable by altering the two amino acids flanking the peptide bond cleaved by a naturally-occurring protease. In other aspects of this embodiment, a naturally-occurring protease cleavage site is made inoperable by altering, e.g., at least three amino acids including the two amino acids flanking the peptide bond cleaved by a naturally-occurring protease; at least four amino acids including the two amino acids flanking the peptide bond cleaved by a naturally-occurring protease; at least five amino acids including the two amino acids flanking the peptide bond cleaved by a naturally-occurring protease; at least six amino acids including the two amino acids flanking the peptide bond cleaved by a naturally-occurring protease; at least seven amino acids including the two amino acids flanking the peptide bond cleaved by a naturally-occurring protease; at least eight amino acids including the two amino acids flanking the peptide bond cleaved by a naturally-occurring protease; at least nine amino acids including the two amino acids flanking the peptide bond cleaved by a naturally-occurring protease; at least ten amino acids including the two amino acids flanking the peptide bond cleaved by a naturally-occurring protease; at least 15 amino acids including the two amino acids flanking the peptide bond cleaved by a naturally-occurring protease; or at least 20 amino acids including the two amino acids flanking the peptide bond cleaved by a naturally-occurring protease.

In still other aspects of this embodiment, a naturally-occurring di-chain protease cleavage site is made inoperable by altering, e.g., at most three amino acids including the two amino acids flanking the peptide bond cleaved by a naturally-occurring protease; at most four amino acids including the two amino acids flanking the peptide bond cleaved by a naturally-occurring protease; at most five amino acids including the two amino acids flanking the peptide bond cleaved by a naturally-occurring protease; at most six amino acids including the two amino acids flanking the peptide bond cleaved by a naturally-occurring protease; at most seven amino acids including the two amino acids flanking the peptide bond cleaved by a naturally-occurring protease; at most eight amino acids including the two amino acids flanking the peptide bond cleaved by a naturally-occurring protease; at most nine amino acids including the two amino acids flanking the peptide bond cleaved by a naturally-occurring protease; at most ten amino acids including the two amino acids flanking the peptide bond cleaved by a naturally-occurring protease; at most 15 amino acids including the two amino acids flanking the peptide bond cleaved by a naturally-occurring protease; or at most 20 amino acids including the two amino acids flanking the peptide bond cleaved by a naturally-occurring protease.

It is understood that a TVEMP disclosed in the present specification can optionally further comprise a flexible region comprising a flexible spacer. A flexible region comprising flexible spacers can be used to adjust the length of a polypeptide region in order to optimize a characteristic, attribute or property of a polypeptide. As a non-limiting example, a polypeptide region comprising one or more flexible spacers in tandem can be use to better expose a protease cleavage site thereby facilitating cleavage of that site by a protease. As another non-limiting example, a polypeptide region comprising one or more flexible spacers in tandem can be use to better present a retargeted peptide binding domain, thereby facilitating the binding of that binding domain to its receptor.

A flexible space comprising a peptide is at least one amino acid in length and comprises non-charged amino acids with small side-chain R groups, such as, e.g., glycine, alanine, valine, leucine or serine. Thus, in an embodiment a flexible spacer can have a length of, e.g., at least 1, 2, 3, 4, 5, 6, 7, 8, 9, or 10 amino acids; or at most 1, 2, 3, 4, 5, 6, 7, 8, 9, or 10 amino acids. In still another embodiment, a flexible spacer can be, e.g., between 1-3 amino acids, between 2-4 amino acids, between 3-5 amino acids, between 4-6 amino acids, or between 5-7 amino acids. Non-limiting examples of a flexible spacer include, e.g., a G-spacers such as GGG, GGGG (SEQ ID NO: 63), and GGGGS (SEQ ID NO: 64) or an A-spacers such as AAA, AAAA (SEQ ID NO: 65) and AAAAV (SEQ ID NO: 66). Such a flexible region is operably-linked in-frame to the TVEMP as a fusion protein.

Thus, in an embodiment, a TVEMP disclosed in the present specification can further comprise a flexible region comprising a flexible spacer. In another embodiment, a TVEMP disclosed in the present specification can further comprise flexible region comprising a plurality of flexible spacers in tandem. In aspects of this embodiment, a flexible region can comprise in tandem, e.g., at least 1, 2, 3, 4, or 5 G-spacers; or at most 1, 2, 3, 4, or 5 G-spacers. In still other aspects of this embodiment, a flexible region can comprise in tandem, e.g., at least 1, 2, 3, 4, or 5 A-spacers; or at most 1, 2, 3, 4, or 5 A-spacers. In another aspect of this embodiment, a TVEMP can comprise a flexible region comprising one or more copies of the same flexible spacers, one or more copies of different flexible-spacer regions, or any combination thereof.

In other aspects of this embodiment, a TVEMP comprising a flexible spacer can be, e.g., a modified BoNT/A, a modified BoNT/B, a modified BoNT/C1, a modified BoNT/D, a modified BoNT/E, a modified BoNT/F, a modified BoNT/G, a modified TeNT, a modified BaNT, or a modified BuNT.

It is envisioned that a TVEMP disclosed in the present specification can comprise a flexible spacer in any and all locations with the proviso that TVEMP is capable of performing the intoxication process. In aspects of this embodiment, a flexible spacer is positioned between, e.g., an enzymatic domain and a translocation domain, an enzymatic domain and a retargeted peptide binding domain, an enzymatic domain and an exogenous protease cleavage site. In other aspects of this embodiment, a G-spacer is positioned between, e.g., an enzymatic domain and a translocation domain, an enzymatic domain and a retargeted peptide binding domain, an enzymatic domain and an exogenous protease cleavage site. In other aspects of this embodiment, an A-spacer is positioned between, e.g., an enzymatic domain and a translocation domain, an enzymatic domain and a retargeted peptide binding domain, an enzymatic domain and an exogenous protease cleavage site.

In other aspects of this embodiment, a flexible spacer is positioned between, e.g., a retargeted peptide binding domain and a translocation domain, a retargeted peptide binding domain and an enzymatic domain, a retargeted peptide binding domain and an exogenous protease cleavage site. In other aspects of this embodiment, a G-spacer is positioned between, e.g., a retargeted peptide binding domain and a translocation domain, a retargeted peptide binding domain and an enzymatic domain, a retargeted peptide binding domain and an exogenous protease cleavage site. In other aspects of this embodiment, an A-spacer is positioned between, e.g., a retargeted peptide binding domain and a translocation domain, a retargeted peptide binding domain and an enzymatic domain, a retargeted peptide binding domain and an exogenous protease cleavage site.

In yet other aspects of this embodiment, a flexible spacer is positioned between, e.g., a translocation domain and an enzymatic domain, a translocation domain and a retargeted peptide binding domain, a translocation domain and an exogenous protease cleavage site. In other aspects of this embodiment, a G-spacer is positioned between, e.g., a translocation domain and an enzymatic domain, a translocation domain and a retargeted peptide binding domain, a translocation domain and an exogenous protease cleavage site. In other aspects of this embodiment, an A-spacer is positioned between, e.g., a translocation domain and an enzymatic domain, a translocation domain and a retargeted peptide binding domain, a translocation domain and an exogenous protease cleavage site.

It is envisioned that a TVEMP disclosed in the present specification can comprise a retargeted peptide binding domain in any and all locations with the proviso that TVEMP is capable of performing the intoxication process. Non-limiting examples include, locating a retargeted peptide binding domain at the amino terminus of a TVEMP; locating a retargeted peptide binding domain between a Clostridial toxin enzymatic domain and a translocation domain of a TVEMP; and locating a retargeted peptide binding domain at the carboxyl terminus of a TVEMP. Other non-limiting examples include, locating a retargeted peptide binding domain between a Clostridial toxin enzymatic domain and a Clostridial toxin translocation domain of a TVEMP. The enzymatic domain of naturally-occurring Clostridial toxins contains the native start methionine. Thus, in domain organizations where the enzymatic domain is not in the amino-terminal location an amino acid sequence comprising the start methionine should be placed in front of the amino-terminal domain. Likewise, where a retargeted peptide binding domain is in the amino-terminal position, an amino acid sequence comprising a start methionine and a protease cleavage site may be operably-linked in situations in which a retargeted peptide binding domain requires a free amino terminus, see, e.g., Shengwen Li et al., Degradable Clostridial Toxins, U.S. patent application Ser. No. 11/572,512 (Jan. 23, 2007), which is hereby incorporated by reference in its entirety. In addition, it is known in the art that when adding a polypeptide that is operably-linked to the amino terminus of another polypeptide comprising the start methionine that the original methionine residue can be deleted.

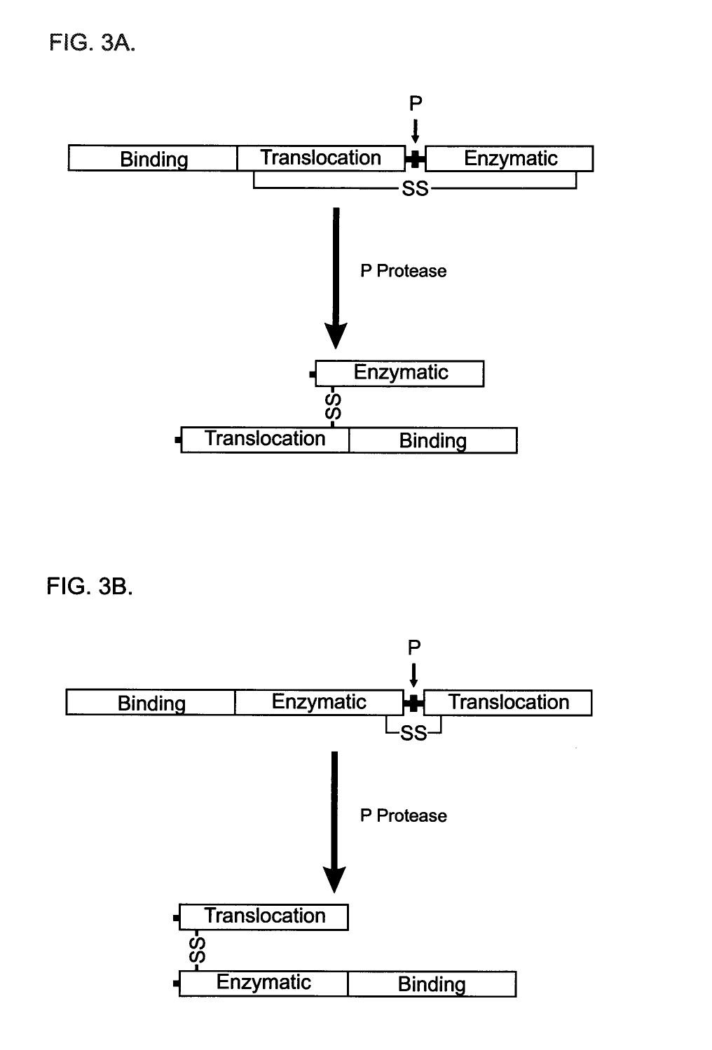

Thus, in an embodiment, a TVEMP can comprise an amino to carboxyl single polypeptide linear order comprising a retargeted peptide binding domain, a translocation domain, an exogenous protease cleavage site and an enzymatic domain (FIG. 3A). In an aspect of this embodiment, a TVEMP can comprise an amino to carboxyl single polypeptide linear order comprising a retargeted peptide binding domain, a Clostridial toxin translocation domain, an exogenous protease cleavage site and a Clostridial toxin enzymatic domain.

In another embodiment, a TVEMP can comprise an amino to carboxyl single polypeptide linear order comprising a retargeted peptide binding domain, an enzymatic domain, an exogenous protease cleavage site, and a translocation domain (FIG. 3B). In an aspect of this embodiment, a TVEMP can comprise an amino to carboxyl single polypeptide linear order comprising a retargeted peptide binding domain, a Clostridial toxin enzymatic domain, an exogenous protease cleavage site, a Clostridial toxin translocation domain.

In yet another embodiment, a TVEMP can comprise an amino to carboxyl single polypeptide linear order comprising an enzymatic domain, an exogenous protease cleavage site, a retargeted peptide binding domain, and a translocation domain (FIG. 4A). In an aspect of this embodiment, a TVEMP can comprise an amino to carboxyl single polypeptide linear order comprising a Clostridial toxin enzymatic domain, an exogenous protease cleavage site, a retargeted peptide binding domain, and a Clostridial toxin translocation domain.

In yet another embodiment, a TVEMP can comprise an amino to carboxyl single polypeptide linear order comprising a translocation domain, an exogenous protease cleavage site, a retargeted peptide binding domain, and an enzymatic domain (FIG. 4B). In an aspect of this embodiment, a TVEMP can comprise an amino to carboxyl single polypeptide linear order comprising a Clostridial toxin translocation domain, a retargeted peptide binding domain, an exogenous protease cleavage site and a Clostridial toxin enzymatic domain.

In another embodiment, a TVEMP can comprise an amino to carboxyl single polypeptide linear order comprising an enzymatic domain, a retargeted peptide binding domain, an exogenous protease cleavage site, and a translocation domain (FIG. 4C). In an aspect of this embodiment, a TVEMP can comprise an amino to carboxyl single polypeptide linear order comprising a Clostridial toxin enzymatic domain, a retargeted peptide binding domain, an exogenous protease cleavage site, a Clostridial toxin translocation domain.

In yet another embodiment, a TVEMP can comprise an amino to carboxyl single polypeptide linear order comprising a translocation domain, a retargeted peptide binding domain, an exogenous protease cleavage site and an enzymatic domain (FIG. 4D). In an aspect of this embodiment, a TVEMP can comprise an amino to carboxyl single polypeptide linear order comprising a Clostridial toxin translocation domain, a retargeted peptide binding domain, an exogenous protease cleavage site and a Clostridial toxin enzymatic domain.

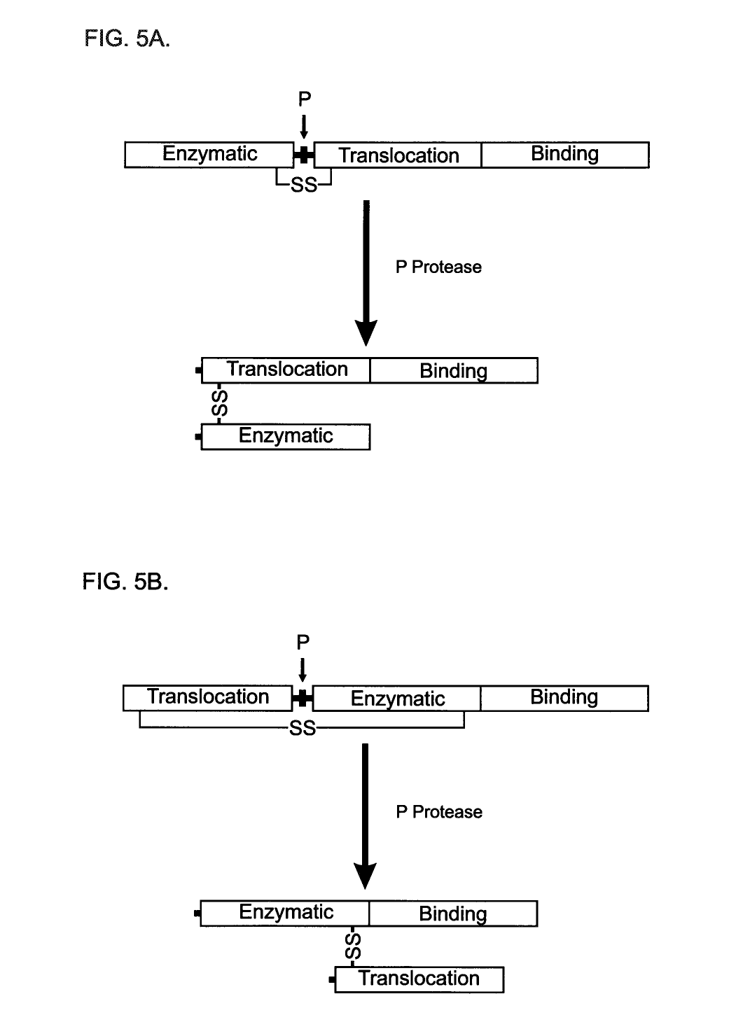

In still another embodiment, a TVEMP can comprise an amino to carboxyl single polypeptide linear order comprising an enzymatic domain, an exogenous protease cleavage site, a translocation domain, and a retargeted peptide binding domain (FIG. 5A). In an aspect of this embodiment, a TVEMP can comprise an amino to carboxyl single polypeptide linear order comprising a Clostridial toxin enzymatic domain, an exogenous protease cleavage site, a Clostridial toxin translocation domain, and a retargeted peptide binding domain.

In still another embodiment, a TVEMP can comprise an amino to carboxyl single polypeptide linear order comprising a translocation domain, an exogenous protease cleavage site, an enzymatic domain and a retargeted peptide binding domain, (FIG. 5B). In an aspect of this embodiment, a TVEMP can comprise an amino to carboxyl single polypeptide linear order comprising a Clostridial toxin translocation domain, a retargeted peptide binding domain, an exogenous protease cleavage site and a Clostridial toxin enzymatic domain.

A composition useful in the invention generally is administered as a pharmaceutical acceptable composition comprising a TVEMP. As used herein, the term “pharmaceutically acceptable” refers to any molecular entity or composition that does not produce an adverse, allergic or other untoward or unwanted reaction when administered to an individual. As used herein, the term “pharmaceutically acceptable composition” is synonymous with “pharmaceutical composition” and refers to a therapeutically effective concentration of an active ingredient, such as, e.g., any of the TVEMPs disclosed in the present specification. A pharmaceutical composition comprising a TVEMP is useful for medical and veterinary applications. A pharmaceutical composition may be administered to a patient alone, or in combination with other supplementary active ingredients, agents, drugs or hormones. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilizate, tablet, pill, pellet, capsule, powder, syrup, elixir or any other dosage form suitable for administration.

Aspects of the present invention provide, in part, a composition comprising a TVEMP. It is envisioned that any of the composition disclosed in the present specification can be useful in a method of treating urogenital-neurological disorder in a mammal in need thereof, with the proviso that the composition prevents or reduces a symptom associated with the urogenital-neurological disorder. Non-limiting examples of compositions comprising a TVEMP include a TVEMP comprising a retargeted peptide binding domain, a Clostridial toxin translocation domain and a Clostridial toxin enzymatic domain. It is envisioned that any TVEMP disclosed in the present specification can be used, including those disclosed in, e.g., Steward, supra, (2007); Dolly, supra, (2007); Foster, supra, WO 2006/059093 (2006); Foster, supra, WO 2006/059105 (Jun. 8, 2006). It is also understood that the two or more different TVEMPs can be provided as separate compositions or as part of a single composition.

It is also envisioned that a pharmaceutical composition comprising a TVEMP can optionally include a pharmaceutically acceptable carriers that facilitate processing of an active ingredient into pharmaceutically acceptable compositions. As used herein, the term “pharmacologically acceptable carrier” is synonymous with “pharmacological carrier” and refers to any carrier that has substantially no long term or permanent detrimental effect when administered and encompasses terms such as “pharmacologically acceptable vehicle, stabilizer, diluent, additive, auxiliary or excipient.” Such a carrier generally is mixed with an active compound, or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, e.g., water, saline, glycine, hyaluronic acid and the like; solid carriers such as, e.g., mannitol, lactose, starch, magnesium stearate, sodium saccharin, talcum, cellulose, glucose, sucrose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in PHARMACEUTICAL DOSAGE FORMS AND DRUG DELIVERY SYSTEMS (Howard C. Ansel et al., eds., Lippincott Williams & Wilkins Publishers, 7^{th} ed. 1999); REMINGTON: THE SCIENCE AND PRACTICE OF PHARMACY (Alfonso R. Gennaro ed., Lippincott, Williams & Wilkins, 20^{th} ed. 2000); GOODMAN & GILMAN'S THE PHARMACOLOGICAL BASIS OF THERAPEUTICS (Joel G. Hardman et al., eds., McGraw-Hill Professional, 10^{th} ed. 2001); and HANDBOOK OF PHARMACEUTICAL EXCIPIENTS (Raymond C. Rowe et al., APhA Publications, 4^{th} edition 2003). These protocols are routine procedures and any modifications are well within the scope of one skilled in the art and from the teaching herein.

It is further envisioned that a pharmaceutical composition disclosed in the present specification can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening or flavoring agents, and the like. Various buffers and methods for adjusting pH can be used to prepare a pharmaceutical composition disclosed in the present specification, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate, a stabilized oxy chloro composition, such as, e.g., PURITE® and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition useful in the invention.

In an embodiment, a composition comprising a TVEMP is a pharmaceutical composition comprising a TVEMP. In aspects of this embodiment, a pharmaceutical composition comprising a TVEMP further comprises a pharmacological carrier, a pharmaceutical component, or both a pharmacological carrier and a pharmaceutical component. In other aspects of this embodiment, a pharmaceutical composition comprising a TVEMP further comprises at least one pharmacological carrier, at least one pharmaceutical component, or at least one pharmacological carrier and at least one pharmaceutical component.

Aspects of the present invention provide, in part, an urogenital-neurological disorder. As used herein, the term “urogenital-neurological disorder” refers to an urogenital-rooted disorder where at least one of the underlying symptoms being treated is due to a nerve-based etiology, such as, e.g., a spastic dysfunction and/or degeneration of the sacral reflex arcs. Non-limiting examples of urogenital-neurological disorders, include, without limitation, urinary incontinence, overactive bladder, detrusor dysfunction, lower urinary tract dysfunction, urinary retention, urinary hesitancy, polyuria, nocturia, chronic urinary tract infection, prostate disorders associated with or without other urogenital disorders, uterine disorders associated with or without other urogenital disorders, and urogenital disorders associated with neurogenic dysfunction (such as, e.g., urogenital disorders associated with Parkinson's Disease, multiple sclerosis, spina bifida, transverse myelitis, stroke, spinal cord injury, spasm reflex, and a neurologic lesion of the spinal cord or brain), and other such urogenital disorders of a nerve-based etiology.

An individual's ability to hold urine and maintain continence depends on normal function of the lower urinary tract, the kidneys, and the nervous system. The individual must also have a physical and psychological ability to recognize and appropriately respond to the urge to urinate. The bladders ability to fill and store urine requires a functional sphincter muscle (which controls the flow of urine out of the body) and a stable bladder wall muscle (detrusor). Normal bladder function is dependent on the nerves that sense the fullness of the bladder and on those that trigger the muscle movements that either empty it or retain urine. The process of urination involves two phases: 1) filling and storage of bladder and 2) emptying of bladder. During the filling and storage phase, the bladder stretches so it can hold the increasing amount of urine. The bladder of an average person can hold 350 mL to 550 mL of urine. Generally, the reflex to urinate is triggered when the bladder of an individual when approximately 200 mL of urine collects in the bladder. The emptying phase requires that the detrusor muscle contract, forcing urine out of the bladder through the urethra. The sphincter muscle must relax at the same time, so that urine can flow out of the body. The bladder, internal sphincters, and external sphincters may all be affected by nerve-based disorders that create abnormalities in bladder function. The damage can cause the bladder to be underactive, in which it is unable to contract and unable to empty completely, or it can be overactive, in which it contracts too quickly or frequently.

One type of urogenital-neurological disorder is urinary incontinence. Urinary incontinence is the inability to control the passage of urine. This can range from an occasional leakage of urine, to a complete inability to hold any urine. Urinary incontinence can be caused by abnormalities in bladder capacity or malfunction of control mechanisms such as the bladder neck and/or external urethral sphincter muscle that are important for the bladders storage function. The many types of urinary incontinence.

Stress incontinence is a type of urinary incontinence in which the strength of the muscles (urethral sphincter) that help control urination is reduced as a result of weakened pelvic muscles that support the bladder and urethra or because of malfunction of the urethral sphincter. The weakness may be caused by prior injury to the urethral area, neurological injury, some medications, or after surgery of the prostate or pelvic area. The sphincter is not able to prevent urine flow when there is increased pressure from the abdomen such as during certain activities like coughing, sneezing, laughing, or exercise. Stress urinary incontinence is the most common type of urinary incontinence in women. Studies have shown about 50% of all women have occasional urinary incontinence, and as many as 10% have frequent incontinence. Nearly 20% of women over age 75 experience daily urinary incontinence. Stress incontinence is often seen in women who have had multiple pregnancies and vaginal childbirths, whose bladder, urethra, or rectal wall stick out into the vaginal space (pelvic prolapse).

Urge incontinence is a type of urinary incontinence that involves a strong, sudden need to urinate, followed by instant bladder contraction and involuntary loss of urine which results in leakage. There is not enough time between when an individual suffering from urge incontinence recognizes the need to urinate and when urination actually occurs. Urge incontinence is leakage of urine due to bladder muscles that contract inappropriately. Often these contractions occur regardless of the amount of urine that is in the bladder. Urge incontinence may result from neurological injuries (such as spinal cord injury or stroke), neurological dysfunction (such as, e.g., Parkinson's Disease and multiple sclerosis), infection, bladder cancer, bladder stones, bladder inflammation, or bladder outlet obstruction. In men, urge incontinence may be due to neurological disease or bladder changes caused by benign prostatic hypertrophy (BPH) or bladder outlet obstruction from an enlarged prostate. The majority of cases of urge incontinence are idiopathic, meaning a specific cause cannot be identified. Although urge incontinence may occur in anyone at any age, it is more common in women and the elderly. Urge incontinence is also known as irritable bladder, spasmodic bladder, and unstable bladder.

Overflow urinary incontinence happens when small amounts of urine leak from a bladder that is always full. In older men, this can occur when the flow of urine from the bladder is blocked, usually by an enlarged prostate. It can sometimes be prevented by medication when early symptoms of prostate enlargement, such as frequent urination, appear. Some people with diabetes also have overflow incontinence. Mixed urinary incontinence describes a disorder where an individual exhibits symptoms associated with both stress incontinence and urge incontinence. Continuous urinary incontinence is the complaint of continuous leakage.

Thus, in an embodiment, a mammal suffering from urinary incontinence is treated with a composition comprising a therapeutically effective amount of a TVEMP where such administration reduces a symptom associated with the urinary incontinence. In an aspect of this embodiment, a mammal suffering from stress incontinence is treated with a composition comprising a therapeutically effective amount of a TVEMP where such administration reduces a symptom associated with the stress incontinence. In another aspect of this embodiment, a mammal suffering from urge incontinence is treated with a composition comprising a therapeutically effective amount of a TVEMP where such administration reduces a symptom associated with the urge incontinence. In still another aspect of this embodiment, a mammal suffering from overflow urinary incontinence is treated with a composition comprising a therapeutically effective amount of a TVEMP where such administration reduces a symptom associated with the overflow urinary incontinence. In a further aspect of this embodiment, a mammal suffering from mixed urinary incontinence is treated with a composition comprising a therapeutically effective amount of a TVEMP where such administration reduces a symptom associated with the mixed urinary incontinence. In a further aspect of this embodiment, a mammal suffering from continuous urinary incontinence is treated with a composition comprising a therapeutically effective amount of a TVEMP where such administration reduces a symptom associated with the continuous urinary incontinence.

Another type of urogenital-neurological disorder is overactive bladder. Overactive bladder is increased urinary urgency, with or without urge urinary incontinence, usually with frequency and nocturia. The individual may report symptoms of urinary urgency (the sudden, intense desire to urinate immediately), urinary frequency (the need to urinate more times than is normal), enuresis (any involuntary loss of urine), polyuria, nocturia, and/or urinary incontinence. Thus, overactive bladder describes a bladder that contracts more often than it should, so that a person feels the need to urinate more frequently and/or urgently than necessary and is characterized by uncontrolled, frequent expulsion of urine from the bladder. An overactive bladder usually, but not always, causes urinary incontinence. Individuals with overactive bladder may go to the bathroom very often, e.g., every two hours during the day and night, and may even wet the bed. Often, a strong urge to void is experienced when only a small amount of urine is in the bladder. There may be reduced bladder capacity and incomplete emptying of urine. An overactive bladder can be caused by interruptions in the nerve pathways to the bladder occurring above the sacrum. For example, spastic bladder may be caused by an inability of the detrusor muscle of the bladder to inhibit emptying contractions until a reasonable amount of urine has accumulated. As such, overactive bladder is often associated with detrusor overactivity, a pattern of bladder muscle contraction observed during urodynamics. Overactive bladder can also be caused by urinary tract infection, outflow obstruction and stress incontinence. Sometimes no cause is found, and such idiopathic cases may be due to anxiety or aging. Symptoms include the need to urinate may times throughout the day and night, the sensation of having to urinate immediately, and/or the sudden leakage of urine from the bladder.

Diseases extrinsic to the bladder may also cause the symptoms of overactive bladder. In the male patient, the extrinsic disorder most often responsible for overactive bladder is bladder outlet obstruction (BOO). Disorders extrinsic to the bladder in the female patient include urethral diverticulum, retroverted uterus, pelvic prolapse (including cystocele), gravid uterus, and loss or reduction of estrogen. Disorders extrinsic to the bladder common to both men and woman include pelvic mass, physiologic nocturnal diuresis, and polyuria caused by factors such as excessive fluid intake, diuretic use, or diabetes. Neuromuscular disorders may also account for the overactive bladder. Neurogenic disorders resulting from nerve damage can also cause overactive bladder, including, without limitation, Parkinson disease, multiple sclerosis, spina bifida, cervical stenosis, spinal cord injury, diabetic neuropathy, pelvic surgery, or invertebral disc herniation, hydrocephalus, stroke, spinal cord injuries and lesions of the spinal cord or brain. Bladder aging may also account for these symptoms. A patient history of pelvic trauma, pelvic radiation, or bladder, prostate, or urethral surgery should also be considered when seeking to determine the etiology of the overactive bladder.

Thus, in an embodiment, a mammal suffering from overactive bladder is treated with a composition comprising a therapeutically effective amount of a TVEMP where such administration reduces a symptom associated with the overactive bladder. In an aspect of this embodiment, a mammal suffering from overactive bladder is treated with a composition comprising a therapeutically effective amount of a TVEMP where such administration reduces incontinence, reduces urinary frequency, reduces urinary urgency, reduces enuresis, reduces polyuria, reduces nocturia, and/or reduces urinary incontinence.

Another type of urogenital-neurological disorder is detrusor dysfunction, including, without limitation, detrusor overactivity, detrusor instability, and detrusor-sphincter dyssynergia. One kind of detrusor dysfunction is detrusor overactivity or involuntary detrusor contractions (previously termed detrusor hyperreflexia). Detrusor overactivity involves increased involuntary contractions of the detrusor muscle during the filling phase which may be spontaneous or provoked resulting in uninhibitable bladder contractions. The muscle contraction patterns of detrusor overactivity include, without limitation, phasic detrusor overactivity and terminal detrusor overactivity. Detrusor overactivity can be either idiopathic in nature or they can be caused by non-neurogenic or neurogenic conditions. Symptoms of detrusor overactivity include, without limitation, uninhibitable bladder contractions, urinary urgency, urinary frequency, enuresis, polyuria, nocturia, and/or urinary incontinence. Another kind of detrusor dysfunction is detrusor instability. Detrusor instability involves uncontrolled involuntary contractions of the detrusor muscle resulting in uninhibitable bladder contractions irrespective of bladder capacity. Symptoms of detrusor instability include, without limitation, uninhibitable bladder contractions, urinary urgency, urinary frequency, enuresis, polyuria, nocturia, and/or urinary incontinence. Another kind of detrusor dysfunction is detrusor-sphincter dyssynergia (DSD). Detrusor-sphincter dyssynergia occurs when the contraction of the detrusor musculature is not coordinated with the relaxation of the sphincter thereby preventing the urethra from relaxing completely during voiding. Symptoms of detrusor-sphincter dyssynergia include, without limitation, urine flow interruption, raised detrusor pressure and/or urinary retention. DSD can be caused as a consequence of a neurological condition such as spinal injury or multiple sclerosis.

Thus, in an embodiment, a mammal suffering from detrusor dysfunction is treated with a composition comprising a therapeutically effective amount of a TVEMP where such administration reduces a symptom associated with the detrusor dysfunction. In an aspect of this embodiment, a mammal suffering from detrusor dysfunction is treated with a composition comprising a therapeutically effective amount of a TVEMP where such administration reduces uninhibitable bladder contractions, reduces urinary frequency, reduces urinary urgency, reduces enuresis, reduces polyuria, reduces nocturia, reduces urinary incontinence, reduces urine flow interruption, reduces detrusor pressure, and/or reduces urinary retention.

In another embodiment, a mammal suffering from detrusor overactivity is treated with a composition comprising a therapeutically effective amount of a TVEMP where such administration reduces a symptom associated with the detrusor overactivity. In an aspect of this embodiment, a mammal suffering from detrusor overactivity is treated with a composition comprising a therapeutically effective amount of a TVEMP where such administration reduces uninhibitable bladder contractions, reduces urinary frequency, reduces urinary urgency, reduces enuresis, reduces polyuria, reduces nocturia, and/or reduces urinary incontinence.

In yet another embodiment, a mammal suffering from detrusor instability is treated with a composition comprising a therapeutically effective amount of a TVEMP where such administration reduces a symptom associated with the detrusor instability. In an aspect of this embodiment, a mammal suffering from detrusor instability is treated with a composition comprising a therapeutically effective amount of a TVEMP where such administration reduces uninhibitable bladder contractions, reduces urinary frequency, reduces urinary urgency, reduces enuresis, reduces polyuria, reduces nocturia, and/or reduces urinary incontinence.

In still another embodiment, a mammal suffering from detrusor-sphincter dyssynergia is treated with a composition comprising a therapeutically effective amount of a TVEMP where such administration reduces a symptom associated with the detrusor-sphincter dyssynergia. In an aspect of this embodiment, a mammal suffering from detrusor-sphincter dyssynergia is treated with a composition comprising a therapeutically effective amount of a TVEMP where such administration reduces urine flow interruption, reduces detrusor pressure, and/or reduces urinary retention.

Another type of urogenital-neurological disorder is a lower urinary tract dysfunction (LUTD). See e.g., Paul Abrams et al., The Standardisation of Terminology of Lower Urinary Tract Function: Reposrt from the Standardisation Subcommittee of the International Continence Society, 21 Neurourol Urodyn. 167-178 (2002), which is hereby incorporated by reference in its entirety. Lower urinary tract dysfunctions manifest three general types of symptoms: storage, voiding, and post-micturition symptoms. Storage symptoms are experienced during the storage phase of the bladder and include, without limitation, urinary urgency, urinary frequency, enuresis, polyuria, nocturia increased bladder sensation, decreased bladder sensation, absent bladder sensation, non-specific bladder sensation, and/or urinary incontinence. Voiding symptoms are experienced during the voiding phase. Symptoms include, without limitation, reduced urine flow, splitting or spraying of urine, intermittent urine flow, urinary hesitancy, strained effort to void urine, and/or terminal dribble of urine. Post-micturition symptoms are experienced immediately after micturition and include, without limitation, sensation of incomplete emptying and/or post-micturition dribble.

Thus, in an embodiment, a mammal suffering from a lower urinary tract dysfunction is treated with a composition comprising a therapeutically effective amount of a TVEMP where such administration reduces a symptom associated with the lower urinary tract dysfunction. In an aspect of this embodiment, a mammal suffering from a lower urinary tract dysfunction is treated with a composition comprising a therapeutically effective amount of a TVEMP where such administration reduces storage symptoms. In aspects of this embodiment, the storage symptom reduced is urinary urgency, urinary frequency, enuresis, polyuria, nocturia increased bladder sensation, decreased bladder sensation, absent bladder sensation, non-specific bladder sensation, or urinary incontinence. In another aspect of this embodiment, a mammal suffering from a lower urinary tract dysfunction is treated with a composition comprising a therapeutically effective amount of a TVEMP where such administration reduces voiding symptoms. In aspects of this embodiment, the voiding symptom reduced is reduced urine flow, splitting or spraying of urine, intermittent urine flow, urinary hesitancy, strained effort to void urine, or terminal dribble of urine. In yet another aspect of this embodiment, a mammal suffering from a lower urinary tract dysfunction is treated with a composition comprising a therapeutically effective amount of a TVEMP where such administration reduces post-micturition symptoms. In aspects of this embodiment, the post-micturition symptom reduced is sensation of incomplete emptying or post-micturition dribble.

Another type of urogenital-neurological disorder is urinary retention. Urinary retention is the inability to pass urine from the bladder and may be either an acute or chronic condition. Normally, the reflex to urinate is triggered when the bladder fills to approximately 300-500 mL. The bladder is then emptied when the contraction of the bladder wall forces urine out through the urethra. The bladder, internal sphincters, and external sphincters may all be affected by disorders that create abnormalities in bladder function resulting in urinary retention. Urinary retention can result either from loss of bladder muscle contracting performance or loss of appropriate coordination between the bladder muscle and the urethral sphincter muscle. The inability to properly relax the urinary sphincter muscles causing difficulty in emptying the bladder, which can lead to urinary retention. Often, a strong urge to void is experienced when only a small amount of urine is in the bladder. In addition, there may be reduced bladder capacity and incomplete emptying of urine. Urinary retention may also be caused by difficulty in relaxing the urinary sphincter muscle because the sphincter may be spastic. Alternatively, the bladder neck may be hypertrophied. Other causes of urinary retention include interruptions in the nerve pathways to the bladder occurring above the sacrum. This nerve damage results in a loss of sensation and motor control and is often seen in stroke, Parkinson's disease, spina bifida, diabetes, pelvic surgery, or invertebral disc herniation, and most forms of spinal cord injuries. Sometimes no cause is found, and such idiopathic cases may be due to anxiety or aging. Urinary retention can also occur by a blockage to the flow of urine due to prostate enlargement or urinary tract stones. Another type of urinary retention disorder is stones, which block the urinary tract of an individual thereby causing stoppage of urine flow and/or infection. Either chronic or acute retention may lead to incontinence due to leakage of urine from an overfull bladder.

Thus, in an embodiment, a mammal suffering from urinary retention is treated with a composition comprising a therapeutically effective amount of a TVEMP where such administration reduces a symptom associated with the urinary retention. In an aspect of this embodiment, a mammal suffering from urinary retention is treated with a composition comprising a therapeutically effective amount of a TVEMP where such administration reduces urinary urgency, reduces urinary frequency, increases bladder capacity, reduces urinary incontinence, and/or restores normal urine flow.

In another embodiment, a mammal suffering from acute urinary retention is treated with a composition comprising a therapeutically effective amount of a TVEMP where such administration reduces a symptom associated with the acute urinary retention. In yet another embodiment, a mammal suffering from chronic urinary retention is treated with a composition comprising a therapeutically effective amount of a TVEMP where such administration reduces a symptom associated with the chronic urinary retention.

Another type of urogenital-neurological disorder is urinary hesitancy. Urinary hesitancy is difficulty starting or maintaining a urinary stream. This problem affects people of all ages and occurs in both sexes, but it is most common in older men with enlarged prostate glands. Urinary hesitancy usually comes on gradually. It sometimes goes unnoticed until urinary retention (complete inability to urinate) produces distention and discomfort in the bladder. Almost all older men have some degree of difficulty in starting urination, dribbling, or decreased force of the urinary stream. Urinary hesitancy can be caused by benign prostatic hyperplasia (enlarged prostate), urinary tract infection, especially if chronic and recurrent, prostatitis (inflammation or infection of the prostate gland), drugs (some cold remedies, some nasal decongestants, tricyclic antidepressants, and anticholinergics which may be used for incontinence), shy or bashful bladder syndrome in younger people (unable to urinate when another person is in the room), and neurological disorders.

Thus, in an embodiment, a mammal suffering from urinary hesitancy is treated with a composition comprising a therapeutically effective amount of a TVEMP where such administration reduces a symptom associated with the urinary hesitancy. In an aspect of this embodiment, a mammal suffering from urinary hesitancy is treated with a composition comprising a therapeutically effective amount of a TVEMP where such administration reduces urinary urgency, reduces urinary frequency, increases bladder capacity, reduces urinary incontinence, and/or restores normal urine flow.

Another type of urogenital-neurological disorder is polyuria. Polyuria is when a person releases abnormally excessive volume of urine each day. An excessive volume of urination for an adult would be at least 2.5 liters of urine per day. Polyuria is a fairly common symptom, which is often noticed when you have to get up to use the bathroom at night. Thus, in an embodiment, a mammal suffering from polyuria is treated with a composition comprising a therapeutically effective amount of a TVEMP where such administration reduces a symptom associated with the polyuria.

Another type of urogenital-neurological disorder is nocturia. Nocturia is excessive urination at night, such as by waking up several times during the night to urinate. Normally, urine decreases in amount and become more concentrated at night, and as such, most people can sleep 6 to 8 hours without having to urinate. But, persons with nocturia get up more than once during the night to urinate. Because of this, those who have excessive urination at night often have disrupted sleep cycles. Causes include benign prostatic hyperplasia, certain drugs including diuretics, cardiac glycosides, demeclocycline, lithium, methoxyflurane, phenytoin, propoxyphene, and excessive vitamin D, chronic or recurrent urinary tract infection, chronic renal failure, congestive heart failure, cystitis, diabetes, drinking too much fluid before bedtime, particularly coffee, caffeinated beverages, or alcohol, and obstructive sleep apnea and other sleeping disorders. Thus, in an embodiment, a mammal suffering from nocturia is treated with a composition comprising a therapeutically effective amount of a TVEMP where such administration reduces a symptom associated with the nocturia.

Another type of urogenital-neurological disorder is chronic urinary tract infection (recurrent infection). Chronic urinary tract infection (UTI) is a bacterial infection of the bladder or lower urinary tract (urethra) that lasts for a long time. Most urinary tract infections occur in the lower urinary tract, which includes the bladder and urethra. The condition occurs when the normally clean lower urinary tract is infected by bacteria and becomes inflamed. Urinary tract infections are very common. Most of the time, symptoms of a urinary tract infection disappear within 24-48 hours after treatment begins. However, if the condition occurs more than twice in 6 months, lasts longer than 2 weeks, or does not respond to usual treatment, it is considered chronic. The elderly are at increased risk for such infections because the bladder doesn't empty fully due to such conditions as benign prostatic hyperplasia, prostatitis, and urethral strictures. Other irritating symptoms may include painful urination (dysuria), which may be a result of a urinary tract infection (UTI) caused by urine being held too long in the bladder. UTI with fever is a sign of potential severe kidney infection (pyelonephritis) and is a more worrisome situation as it may result in permanent damage of the kidney(s). Another type of urinary tract infection is vesicoureteral reflux (VUR). Vesicoureteral reflux is an abnormal backup of urine from the bladder to the kidney(s) that occurs as a way of releasing high pressure within the bladder. A UTI is of particular concern as VUR may place the patient at significant risk for a severe kidney infection by transporting infected bladder urine directly to the kidney(s).

Thus, in an embodiment, a mammal suffering from chronic urinary tract infection is treated with a composition comprising a therapeutically effective amount of a TVEMP where such administration reduces a symptom associated with the chronic urinary tract infection. In another embodiment, a mammal suffering from dysuria is treated with a composition comprising a therapeutically effective amount of a TVEMP where such administration reduces a symptom associated with the dysuria. In yet another embodiment, a mammal suffering from vesicoureteral reflux is treated with a composition comprising a therapeutically effective amount of a TVEMP where such administration reduces a symptom associated with the vesicoureteral reflux.

Other types of urogenital-neurological disorders are disorders associated with prostate disorders. The prostate is a partially glandular and partially fibromuscular organ of the male reproductive system that that produces the fluid that carries sperm during ejaculation. It surrounds the urethra, the tube through which urine passes out of the body. One type of prostate disorder is benign prostatic hyperplasia (BPH). During aging, the prostate tends to enlarge (hypertrophy) and this enlarged prostate is often called benign prostatic hyperplasia (BPH) or benign prostatic hypertrophy. Prostatic enlargement can lead to urethral obstruction and voiding dysfunction because the enlarged gland can press on the urethra. BPH is not cancer, and it does not raise your risk for prostate cancer. One type of prostate disorder is prostatitis. Prostatitis is an inflammation of the prostate gland. Prostatitis includes acute and chronic bacterial prostatitis and inflammation not caused by bacterial infection (abacterial prostatitis). One type of prostate disorder is prostatodynia. Prostatodynia is a type of inflammation of the prostate not due to bacterial infection that may be caused by abnormal nerves or muscles in the region. Prostatodynia is typically a chronic, painful disease. The symptoms (including chills, fever, pain in the lower back and genital area, body aches, burning or painful urination, and the frequent and urgent need to urinate) characteristically go away and then come back without warning.

Thus, in an embodiment, a mammal suffering from a urogenital-neurological disorder associated with a prostate disorder is treated with a composition comprising a therapeutically effective amount of a TVEMP where such administration reduces a symptom associated with the urogenital-neurological disorder associated with the prostate disorder. In another aspect of this embodiment, a mammal suffering from urogenital-neurological disorder associated with benign prostatic hyperplasia is treated with a composition comprising a therapeutically effective amount of a TVEMP where such administration reduces a symptom associated with the urogenital-neurological disorder associated with benign prostatic hyperplasia. In yet another aspect of this embodiment, a mammal suffering from urogenital-neurological disorder associated with prostatitis is treated with a composition comprising a therapeutically effective amount of a TVEMP where such administration reduces a symptom associated with the urogenital-neurological disorder associated with prostatitis. In still another aspect of this embodiment, a mammal suffering from urogenital-neurological disorder associated with prostatodynia is treated with a composition comprising a therapeutically effective amount of a TVEMP where such administration reduces a symptom associated with the urogenital-neurological disorder associated with prostatodynia.

In another embodiment, a mammal suffering from a prostate disorder is treated with a composition comprising a therapeutically effective amount of a TVEMP where such administration reduces a symptom associated with the prostate disorder. In an aspect of this embodiment, a mammal suffering from benign prostatic hyperplasia is treated with a composition comprising a therapeutically effective amount of a TVEMP where such administration reduces a symptom associated with the benign prostatic hyperplasia. In yet another aspect of this embodiment, a mammal suffering from prostatitis is treated with a composition comprising a therapeutically effective amount of a TVEMP where such administration reduces a symptom associated with the prostatitis. In still another aspect of this embodiment, a mammal suffering from prostatodynia is treated with a composition comprising a therapeutically effective amount of a TVEMP where such administration reduces a symptom associated with the prostatodynia.

Other types of urogenital-neurological disorders are disorders associated with uterine disorders. The uterus is a hollow, muscular pear-shaped female reproductive organ in which the fertilized zygote implants and develops into the fetus. The uterus comprises a corpus made up of two layers of tissue, fundus, isthmus, and cervix located between the urinary bladder and the rectum in the pelvic cavity of female mammals. One type of uterine disorder is endometriosis. Endometriosis is a condition in which the tissue that lines the inside of the uterus (called the endometrium or endometrial lining) is found growing in other areas outside of the uterus (commonly the ovaries, fallopian tubes, outer surface of the uterus, outer surface of the intestines, and nearby structures of the pelvis). This condition often causes severe pain within the lower abdomen and pelvis that may be associated with your periods each month. The symptoms of endometriosis include pain before and during menstrual periods, pain at the time of ovulation, pain during or after sexual activity, heavy or irregular bleeding, fatigue, pain with bowel movements at the time of the period, pain with urination. Another type of uterine disorder is dysmenorrhea. Dysmenorrhea is the pain or discomfort (menstrual cramps) during or just before a menstrual period. There are two types of dysmenorrheal, primary dysmenorrhea and secondary dysmenorrhea. Primary dysmenorrhea is severe, disabling cramps without underlying illness. Symptoms may include backache, leg pain, nausea, vomiting, diarrhea, headache, and dizziness. This kind of dysmenorrhea usually affects young woman within two years of the onset of menstruation and lasts one or two days each month. Secondary dysmenorrhea is cramps caused by another medical problem(s) such as endometriosis (abnormalities in the lining of the uterus), adenomyosis (nonmalignant growth of the endometrium into the muscular layer of the uterus), pelvic inflammatory disease, uterine fibroids, cervical narrowing, uterine malposition, pelvic tumors or an IUD (intra-uterine device). This condition usually occurs in older women.

Thus, in an embodiment, a mammal suffering from a urogenital-neurological disorder associated with a uterine disorder is treated with a composition comprising a therapeutically effective amount of a TVEMP where such administration reduces a symptom associated with the urogenital-neurological disorder associated with the uterine disorder. In an aspect of this embodiment, a mammal suffering from endometriosis is treated with a composition comprising a therapeutically effective amount of a TVEMP where such administration reduces a symptom associated with the endometriosis. In an aspect of this embodiment, a mammal suffering from dysmenorrhea is treated with a composition comprising a therapeutically effective amount of a TVEMP where such administration reduces a symptom associated with the dysmenorrhea.

Other types of urogenital-neurological disorders are urogenital-neurological disorders associated with neurogenic dysfunction. Thus, in an embodiment, a mammal suffering from a urogenital-neurological disorder associated with a neurogenic dysfunction is treated with a composition comprising a therapeutically effective amount of a TVEMP where such administration reduces a symptom associated with the urogenital-neurological disorder associated with the neurogenic dysfunction. In an aspect of this embodiment, a mammal suffering from a urogenital-neurological disorder associated with Parkinson's Disease is treated with a composition comprising a therapeutically effective amount of a TVEMP where such administration reduces a symptom associated with the urogenital-neurological disorder associated with Parkinson's Disease. In another aspect of this embodiment, a mammal suffering from a urogenital-neurological disorder associated with multiple sclerosis is treated with a composition comprising a therapeutically effective amount of a TVEMP where such administration reduces a symptom associated with the urogenital-neurological disorder associated with multiple sclerosis. In yet another aspect of this embodiment, a mammal suffering from a urogenital-neurological disorder associated with spina bifida is treated with a composition comprising a therapeutically effective amount of a TVEMP where such administration reduces a symptom associated with the urogenital-neurological disorder associated with spina bifida. In yet another aspect of this embodiment, a mammal suffering from a urogenital-neurological disorder associated with transverse myelitis is treated with a composition comprising a therapeutically effective amount of a TVEMP where such administration reduces a symptom associated with the urogenital-neurological disorder associated with transverse myelitis. In yet another aspect of this embodiment, a mammal suffering from a urogenital-neurological disorder associated with stroke is treated with a composition comprising a therapeutically effective amount of a TVEMP where such administration reduces a symptom associated with the urogenital-neurological disorder associated with stroke. In still another aspect of this embodiment, a mammal suffering from a urogenital-neurological disorder associated with a spinal cord injury is treated with a composition comprising a therapeutically effective amount of a TVEMP where such administration reduces a symptom associated with the urogenital-neurological disorder associated with the spinal cord injury. In still another aspect of this embodiment, a mammal suffering from a urogenital-neurological disorder associated with a spasm reflex is treated with a composition comprising a therapeutically effective amount of a TVEMP where such administration reduces a symptom associated with the urogenital-neurological disorder associated with the spasm reflex. In a further aspect of this embodiment, a mammal suffering from a urogenital-neurological disorder associated with a neurologic lesion of the spinal cord or brain is treated with a composition comprising a therapeutically effective amount of a TVEMP where such administration reduces a symptom associated with the urogenital-neurological disorder associated with the neurologic lesion of the spinal cord or brain.

Aspects of the present invention provide, in part, a mammal. A mammal includes a human, and a human can be a patient. Other aspects of the present invention provide, in part, an individual. An individual includes a human, and a human can be a patient.

Aspects of the present invention provide, in part, administering a composition comprising a TVEMP. As used herein, the term “administering” refers to any delivery mechanism that provides a composition comprising a TVEMP to a patient that potentially results in a clinically, therapeutically, or experimentally beneficial result. A TVEMP can be delivered to a patient using a cellular uptake approach where a TVEMP is delivered intracellular or a gene therapy approach where a TVEMP is express derived from precursor RNAs expressed from an expression vectors.

A composition comprising a TVEMP as disclosed in the present specification can be administered to a mammal using a cellular uptake approach. Administration of a composition comprising a TVEMP using a cellular uptake approach comprise a variety of enteral or parenteral approaches including, without limitation, oral administration in any acceptable form, such as, e.g., tablet, liquid, capsule, powder, or the like; topical administration in any acceptable form, such as, e.g., drops, spray, creams, gels or ointments; intravascular administration in any acceptable form, such as, e.g., intravenous bolus injection, intravenous infusion, intra-arterial bolus injection, intra-arterial infusion and catheter instillation into the vasculature; peri- and intra-tissue administration in any acceptable form, such as, e.g., intraperitoneal injection, intramuscular injection, subcutaneous injection, subcutaneous infusion, intraocular injection, retinal injection, or sub-retinal injection or epidural injection; intravesicular administration in any acceptable form, such as, e.g., catheter instillation; and by placement device, such as, e.g., an implant, a patch, a pellet, a catheter, an osmotic pump, a suppository, a bioerodible delivery system, a non-bioerodible delivery system or another implanted extended or slow release system. An exemplary list of biodegradable polymers and methods of use are described in, e.g., Handbook of Biodegradable Polymers (Abraham J. Domb et al., eds., Overseas Publishers Association, 1997).

A composition comprising a TVEMP can be administered to a mammal by a variety of methods known to those of skill in the art, including, but not restricted to, encapsulation in liposomes, by ionophoresis, or by incorporation into other vehicles, such as hydrogels, cyclodextrins, biodegradable nanocapsules, and bioadhesive microspheres, or by proteinaceous vectors. Delivery mechanisms for administering a composition comprising a TVEMP to a patient are described in, e.g., Leonid Beigelman et al., Compositions for the Delivery of Negatively Charged Molecules, U.S. Pat. No. 6,395,713 (May 28, 2002); and Achim Aigner, Delivery Systems for the Direct Application of siRNAs to Induce RNA Interference (RNAi) in vivo, 2006(716559) J. Biomed. Biotech. 1-15 (2006); Controlled Drug Delivery: Designing Technologies for the Future (Kinam Park & Randy J. Mrsny eds., American Chemical Association, 2000); Vernon G. Wong & Mae W. L. Hu, Methods for Treating Inflammation-mediated Conditions of the Eye, U.S. Pat. No. 6,726,918 (Apr. 27, 2004); David A. Weber et al., Methods and Apparatus for Delivery of Ocular Implants, U.S. Patent Publication No. US2004/0054374 (Mar. 18, 2004); Thierry Nivaggioli et al., Biodegradable Ocular Implant, U.S. Patent Publication No. US2004/0137059 (Jul. 15, 2004); Patrick M. Hughes et al., Anti-Angiogenic Sustained Release Intraocular Implants and Related Methods, U.S. patent application Ser. No. 11/364,687 (Feb. 27, 2006); and Patrick M. Hughes et al., Sustained Release Intraocular Drug Delivery Systems, U.S. Patent Publication 2006/0182783 (Aug. 17, 2006), each of which is hereby incorporated by reference in its entirety.

A composition comprising a TVEMP as disclosed in the present specification can also be administered to a patient using a gene therapy approach by expressing a TVEMP within in a cell manifesting a nerve-based etiology that contributes to a urogenital-neurological disorder. A TVEMP can be expressed from nucleic acid molecules operably-linked to an expression vector, see, e.g., P. D. Good et al., Expression of Small, Therapeutic RNAs in Human Cell Nuclei, 4(1) Gene Ther. 45-54 (1997); James D. Thompson, Polymerase III-based expression of therapeutic RNAs, U.S. Pat. No. 6,852,535 (Feb. 8, 2005); Maciej Wiznerowicz et al., Tuning Silence: Conditional Systems for RNA Interference, 3(9) Nat. Methods 682-688m (2006); Ola Snøve and John J. Rossi, Expressing Short Hairpin RNAi in vivo, 3(9) Nat. Methods 689-698 (2006); and Charles X. Li et al., Delivery of RNA Interference, 5(18) Cell Cycle 2103-2109 (2006). A person of ordinary skill in the art would realize that any TVEMP can be expressed in eukaryotic cells using an appropriate expression vector.

Expression vectors capable of expressing a TVEMP can provide persistent or stable expression of the TVEMP in a cell manifesting a nerve-based etiology that contributes to a urogenital-neurological disorder. Alternatively, expression vectors capable of expressing a TVEMP can provide for transient expression of the TVEMP in a cell manifesting a nerve-based etiology that contributes to a urogenital-neurological disorder. Such transiently expressing vectors can be repeatedly administered as necessary. A TVEMP-expressing vectors can be administered by a delivery mechanism and route of administration discussed above, by administration to target cells ex-planted from a patient followed by reintroduction into the patient, or by any other method that would allow for introduction into the desired target cell, see, e.g., Larry A. Couture and Dan T. Stinchcomb, Anti-gene Therapy: The Use of Ribozymes to Inhibit Gene Function, 12(12) Trends Genet. 510-515 (1996).

The actual delivery mechanism used to administer a composition comprising a TVEMP to a mammal can be determined by a person of ordinary skill in the art by taking into account factors, including, without limitation, the type of urogenital-neurological disorder, the location of the urogenital-neurological disorder, the cause of the urogenital-neurological disorder, the severity of the urogenital-neurological disorder, the degree of relief desired, the duration of relief desired, the particular TVEMP used, the rate of excretion of the TVEMP used, the pharmacodynamics of the TVEMP used, the nature of the other compounds to be included in the composition, the particular route of administration, the particular characteristics, history and risk factors of the patient, such as, e.g., age, weight, general health and the like, or any combination thereof.

In an embodiment, a composition comprising a TVEMP is administered to the site to be treated by injection. In aspects of this embodiment, injection of a composition comprising a TVEMP is by, e.g., intramuscular injection, subdermal injection, or dermal injection. In aspects of this embodiment, injection of a composition comprising a TVEMP is into the lower urinary tract, including the bladder wall, the urinary sphincter or bladder neck.

A composition comprising a TVEMP can be administered to a mammal using a variety of routes. Routes of administration suitable for a method of treating an urogenital-neurological disorder as disclosed in the present specification include both local and systemic administration. Local administration results in significantly more delivery of a composition to a specific location as compared to the entire body of the mammal, whereas, systemic administration results in delivery of a composition to essentially the entire body of the patient. Routes of administration suitable for a method of treating an urogenital-neurological disorder as disclosed in the present specification also include both central and peripheral administration. Central administration results in delivery of a composition to essentially the central nervous system of the patient and includes, e.g., intrathecal administration, epidural administration as well as a cranial injection or implant. Peripheral administration results in delivery of a composition to essentially any area of a patient outside of the central nervous system and encompasses any route of administration other than direct administration to the spine or brain. The actual route of administration of a composition comprising a TVEMP used in a mammal can be determined by a person of ordinary skill in the art by taking into account factors, including, without limitation, the type of urogenital-neurological disorder, the location of the urogenital-neurological disorder, the cause of the urogenital-neurological disorder, the severity of the urogenital-neurological disorder, the degree of relief desired, the duration of relief desired, the particular TVEMP used, the rate of excretion of the TVEMP used, the pharmacodynamics of the TVEMP used, the nature of the other compounds to be included in the composition, the particular route of administration, the particular characteristics, history and risk factors of the mammal, such as, e.g., age, weight, general health and the like, or any combination thereof.

In an embodiment, a composition comprising a TVEMP is administered systemically to a mammal. In another embodiment, a composition comprising a TVEMP is administered locally to a mammal. In an aspect of this embodiment, a composition comprising a TVEMP is administered to the bladder of a mammal. In another aspect of this embodiment, a composition comprising a TVEMP is administered to the prostate of a mammal. In another aspect of this embodiment, a composition comprising a TVEMP is administered to the uterus of a mammal.

Aspects of the present invention provide, in part, administering a therapeutically effective amount of a composition comprising a TVEMP. As used herein, the term “therapeutically effective amount” is synonymous with “therapeutically effective dose” and when used in reference to treating an urogenital-neurological disorder refers to the minimum dose of a TVEMP necessary to achieve the desired therapeutic effect and includes a dose sufficient to reduce a symptom associated with an urogenital-neurological disorder. In aspects of this embodiment, a therapeutically effective amount of a composition comprising a TVEMP reduces a symptom associated with an urogenital-neurological disorder by, e.g., at least 30%, at least 40%, at least 50%, at least 60%, at least 70%, at least 80%, at least 90% or at least 100%. In other aspects of this embodiment, a therapeutically effective amount of a composition comprising a TVEMP reduces a symptom associated with an urogenital-neurological disorder by, e.g., at most 30%, at most 40%, at most 50%, at most 60%, at most 70%, at most 80%, at most 90% or at most 100%. In yet other aspects of this embodiment, a therapeutically effective amount of a composition comprising a TVEMP reduces a symptom associated with an urogenital-neurological disorder by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In other aspects of this embodiment, a therapeutically effective amount of a composition comprising a TVEMP reduces a symptom associated with an urogenital-neurological disorder by, e.g., about one week, about one month, about two months, about three months, about four months, about five months, about six months, about seven months, about eight months, about nine months, about ten months, about eleven months, or about twelve months. In yet other aspects of this embodiment, a therapeutically effective amount of a composition comprising a TVEMP reduces a symptom associated with an urogenital-neurological disorder by, e.g., at least one week, at least one month, at least two months, at least three months, at least four months, at least five months, at least six months, at least seven months, at least eight months, at least nine months, at least ten months, at least eleven months, or at least twelve months. In still other aspects of this embodiment, a therapeutically effective amount of a composition comprising a TVEMP reduces a symptom associated with an urogenital-neurological disorder by, e.g., about 1 week to about three months, about one month to about six months, about one month to about nine months, about one month to about twelve months, about three months to about six months, about three months to about nine months, about three months to about twelve months.

The actual therapeutically effective amount of a composition comprising a TVEMP to be administered to a mammal can be determined by a person of ordinary skill in the art by taking into account factors, including, without limitation, the type of urogenital-neurological disorder, the location of the urogenital-neurological disorder, the cause of the urogenital-neurological disorder, the severity of the urogenital-neurological disorder, the degree of relief desired, the duration of relief desired, the particular TVEMP used, the rate of excretion of the TVEMP used, the pharmacodynamics of the TVEMP used, the nature of the other compounds to be included in the composition, the particular route of administration, the particular characteristics, history and risk factors of the patient, such as, e.g., age, weight, general health and the like, or any combination thereof. Additionally, where repeated administration of a composition comprising a TVEMP is used, the actual effect amount of a composition comprising a TVEMP will further depend upon factors, including, without limitation, the frequency of administration, the half-life of the composition comprising a TVEMP, or any combination thereof. In is known by a person of ordinary skill in the art that an effective amount of a composition comprising a TVEMP can be extrapolated from in vitro assays and in vivo administration studies using animal models prior to administration to humans. Wide variations in the necessary effective amount are to be expected in view of the differing efficiencies of the various routes of administration. For instance, oral administration generally would be expected to require higher dosage levels than administration by intravenous or intravitreal injection. Variations in these dosage levels can be adjusted using standard empirical routines of optimization, which are well-known to a person of ordinary skill in the art. The precise therapeutically effective dosage levels and patterns are preferably determined by the attending physician in consideration of the above-identified factors.

As a non-limiting example, when administering a composition comprising a TVEMP to a mammal, a therapeutically effective amount generally is in the range of about 1 fg to about 3.0 mg. In aspects of this embodiment, an effective amount of a composition comprising a TVEMP can be, e.g., about 100 fg to about 3.0 mg, about 100 pg to about 3.0 mg, about 100 ng to about 3.0 mg, or about 100 μg to about 3.0 mg. In other aspects of this embodiment, an effective amount of a composition comprising a TVEMP can be, e.g., about 100 fg to about 750 μg, about 100 pg to about 750 μg, about 100 ng to about 750 μg, or about 1 μg to about 750 μg. In yet other aspects of this embodiment, a therapeutically effective amount of a composition comprising a TVEMP can be, e.g., at least 1 fg, at least 250 fg, at least 500 fg, at least 750 fg, at least 1 pg, at least 250 pg, at least 500 pg, at least 750 pg, at least 1 ng, at least 250 ng, at least 500 ng, at least 750 ng, at least 1 μg, at least 250 μg, at least 500 μg, at least 750 μg, or at least 1 mg. In still other aspects of this embodiment, a therapeutically effective amount of a composition comprising a TVEMP can be, e.g., at most 1 fg, at most 250 fg, at most 500 fg, at most 750 fg, at most 1 pg, at most 250 pg, at most 500 pg, at most 750 pg, at most 1 ng, at most 250 ng, at most 500 ng, at most 750 ng, at most 1 μg, at least 250 μg, at most 500 μg, at most 750 μg, or at most 1 mg.

As another non-limiting example, when administering a composition comprising a TVEMP to a mammal, a therapeutically effective amount generally is in the range of about 0.00001 mg/kg to about 3.0 mg/kg. In aspects of this embodiment, an effective amount of a composition comprising a TVEMP can be, e.g., about 0.0001 mg/kg to about 0.001 mg/kg, about 0.03 mg/kg to about 3.0 mg/kg, about 0.1 mg/kg to about 3.0 mg/kg, or about 0.3 mg/kg to about 3.0 mg/kg. In yet other aspects of this embodiment, a therapeutically effective amount of a composition comprising a TVEMP can be, e.g., at least 0.00001 mg/kg, at least 0.0001 mg/kg, at least 0.001 mg/kg, at least 0.01 mg/kg, at least 0.1 mg/kg, or at least 1 mg/kg. In yet other aspects of this embodiment, a therapeutically effective amount of a composition comprising a TVEMP can be, e.g., at most 0.00001 mg/kg, at most 0.0001 mg/kg, at most 0.001 mg/kg, at most 0.01 mg/kg, at most 0.1 mg/kg, or at most 1 mg/kg.

Dosing can be single dosage or cumulative (serial dosing), and can be readily determined by one skilled in the art. For instance, treatment of an urogenital-neurological disorder may comprise a one-time administration of an effective dose of a composition comprising a TVEMP. As a non-limiting example, an effective dose of a composition comprising a TVEMP can be administered once to a patient, e.g., as a single injection or deposition at or near the site exhibiting a symptom of an urogenital-neurological disorder. Alternatively, treatment of an urogenital-neurological disorder may comprise multiple administrations of an effective dose of a composition comprising a TVEMP carried out over a range of time periods, such as, e.g., daily, once every few days, weekly, monthly or yearly. As a non-limiting example, a composition comprising a TVEMP can be administered once or twice yearly to a mammal. The timing of administration can vary from mammal to mammal, depending upon such factors as the severity of a mammal's symptoms. For example, an effective dose of a composition comprising a TVEMP can be administered to a mammal once a month for an indefinite period of time, or until the patient no longer requires therapy. A person of ordinary skill in the art will recognize that the condition of the mammal can be monitored throughout the course of treatment and that the effective amount of a composition comprising a TVEMP that is administered can be adjusted accordingly.

A composition comprising a TVEMP as disclosed in the present specification can also be administered to a mammal in combination with other therapeutic compounds to increase the overall therapeutic effect of the treatment. The use of multiple compounds to treat an indication can increase the beneficial effects while reducing the presence of side effects.

Aspects of the present invention can also be described as follows:
1. A method of treating urogenital-neurological disorder in a mammal, the method comprising the step of administering to the mammal in need thereof a therapeutically effective amount of a composition including a TVEMP comprising a retargeted peptide binding domain, a Clostridial toxin translocation domain and a Clostridial toxin enzymatic domain, wherein administration of the composition reduces a symptom of the urogenital-neurological disorder, thereby treating the mammal.
2. The method of 1, wherein the TVEMP comprises a linear amino-to-carboxyl single polypeptide order of 1) the Clostridial toxin enzymatic domain, the Clostridial toxin translocation domain, the retargeted peptide binding domain, 2) the Clostridial toxin enzymatic domain, the retargeted peptide binding domain, the Clostridial toxin translocation domain, 3) the retargeted peptide binding domain, the Clostridial toxin translocation domain, and the Clostridial toxin enzymatic domain, 4) the retargeted peptide binding domain, the Clostridial toxin enzymatic domain, the Clostridial toxin translocation domain, 5) the Clostridial toxin translocation domain, the Clostridial toxin enzymatic domain and the retargeted peptide binding domain, or 6) the Clostridial toxin translocation domain, the retargeted peptide binding domain and the Clostridial toxin enzymatic domain.
3. The method of 1, wherein the retargeted peptide binding domain is an interleukin (IL) peptide binding domain, a vascular endothelial growth factor (VEGF) peptide binding domain, an insulin-like growth factor (IGF) peptide binding domain, or an epidermal growth factor (EGF) peptide binding domain.
4. The method of 3, wherein the interleukin peptide binding domain is an IL-1, an IL-2, an IL-6, an IL-8, an IL-10, or an IL-11.
5. The method of 3, wherein the interleukin peptide binding domain comprises amino acids 123-265 of SEQ ID NO: 67, amino acids 21-153 of SEQ ID NO: 68, amino acids 57-210 of SEQ ID NO: 69, amino acids 21-99 or amino acids 31-94 of SEQ ID NO: 70, amino acids 37-173 or amino acids 19-178 of SEQ ID NO: 71, or amino acids 37-199 of SEQ ID NO: 72.
6. The method of 3, wherein the VEGF peptide binding domain is a VEGF-A, a VEGF-B, a VEGF-C, a VEGF-D, or a placenta growth factor (PIGF).
7. The method of 3, wherein the VEGF peptide binding domain comprises amino acids 50-133 of SEQ ID NO: 73, amino acids 45-127 of SEQ ID NO: 74, amino acids 129-214 of SEQ ID NO: 75, amino acids 109-194 of SEQ ID NO: 76, amino acids 46-163, amino acids 49-162, amino acids 168-345, amino acids 244-306, or amino acids 248-340 of SEQ ID NO: 77, or amino acids 50-131 or amino acids 132-203 of SEQ ID NO: 78.
8. The method of 3, wherein the IGF peptide binding domain is an IGF-1 or an IGF-2.
9. The method of 3, wherein the IGF peptide binding domain comprises amino acids 52-109 or amino acids 49-118 of SEQ ID NO: 79, or amino acids 31-84 or amino acids 25-180 of SEQ ID NO: 80.
10. The method of 3, wherein the EGF peptide binding domain an EGF, a heparin-binding EGF-like growth factor (HB-EGF), a transforming growth factor-α (TGF-α), an amphiregulin (AR), an epiregulin (EPR), an epigen (EPG), a betacellulin (BTC), a neuregulin-1 (NRG1), a neuregulin-2 (NRG2), a neuregulin-3, (NRG3), or a neuregulin-4 (NRG4).
11. The method of 10, wherein the NRG-2 is a NRG-2 isoform 1, a NRG-2 isoform 2, a NRG-2 isoform 3, a NRG-2 isoform 4, a NRG-2 isoform 5, or a NRG-2 isoform 6.
12. The method of 3, wherein the EGF peptide binding domain comprises SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91.
13. The method of 3, wherein the EGF peptide binding domain comprises amino acids 101-251 or amino acids 107-251 of SEQ ID NO: 84, amino acids 63-108 of SEQ ID NO: 85, amino acids 23-154 of SEQ ID NO: 86, amino acids 235-630 of SEQ ID NO: 88, amino acids 398-718 of SEQ ID NO: 89, or amino acids 353-648 of SEQ ID NO: 90.
14. The method of 1, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.
15. The method of 1, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.
16. The method of 1, wherein the urogenital-neurological disorder is urinary incontinence, overactive bladder, detrusor dysfunction, lower urinary tract dysfunction, urinary retention, urinary hesitancy, polyuria, nocturia, chronic urinary tract infection, an urogenital disorder associated with a prostate disorder, an urogenital disorder associated with a uterine disorder, or an urogenital disorder associated with a neurogenic dysfunction.
17. The method of 16, wherein the urinary incontinence is an urge urinary incontinence, a stress urinary incontinence, an overflow urinary incontinence, a mixed urinary incontinence, or a continuous urinary incontinence.
18. The method of 16, wherein the detrusor dysfunction is a detrusor overactivity, a detrusor instability, or a detrusor-sphincter dyssynergia.
19. The method of 16, wherein the urogenital disorder associated with a prostate disorder is an urogenital disorder associated with benign prostatic hyperplasia, an urogenital disorder associated with prostatitis, or an urogenital disorder associated with prostatodynia.
20. The method of 16, wherein the urogenital disorder associated with a neurogenic dysfunction is an urogenital disorder associated with Parkinson's Disease, an urogenital disorder associated with multiple sclerosis, an urogenital disorder associated with spina bifida, an urogenital disorder associated with transverse myelitis, an urogenital disorder associated with stroke, an urogenital disorder associated with a spinal cord injury, an urogenital disorder associated with a spasm reflex, an urogenital disorder associated with a neurologic lesion of the spinal cord, or an urogenital disorder associated with a neurologic lesion of the brain.
21. A method of treating urogenital-neurological disorder in a mammal, the method comprising the step of administering to the mammal in need thereof a therapeutically effective amount of a composition including a TVEMP comprising a retargeted peptide binding domain, a Clostridial toxin translocation domain, a Clostridial toxin enzymatic domain, and an exogenous protease cleavage site, wherein administration of the composition reduces a symptom of the urogenital-neurological disorder, thereby treating the mammal.
22. The method of 21, wherein the TVEMP comprises a linear amino-to-carboxyl single polypeptide order of 1) the Clostridial toxin enzymatic domain, the exogenous protease cleavage site, the Clostridial toxin translocation domain, the retargeted peptide binding domain, 2) the Clostridial toxin enzymatic domain, the exogenous protease cleavage site, the retargeted peptide binding domain, the Clostridial toxin translocation domain, 3) the retargeted peptide binding domain, the Clostridial toxin translocation domain, the exogenous protease cleavage site and the Clostridial toxin enzymatic domain, 4) the retargeted peptide binding domain, the Clostridial toxin enzymatic domain, the exogenous protease cleavage site, the Clostridial toxin translocation domain, 5) the Clostridial toxin translocation domain, the exogenous protease cleavage site, the Clostridial toxin enzymatic domain and the retargeted peptide binding domain, or 6) the Clostridial toxin translocation domain, the exogenous protease cleavage site, the retargeted peptide binding domain and the Clostridial toxin enzymatic domain.
23. The method of 21, wherein the retargeted peptide binding domain is an interleukin (IL) peptide binding domain, a vascular endothelial growth factor (VEGF) peptide binding domain, an insulin-like growth factor (IGF) peptide binding domain, or an epidermal growth factor (EGF) peptide binding domain.
24. The method of 23, wherein the interleukin peptide binding domain is an IL-1, an IL-2, an IL-6, an IL-8, an IL-10, or an IL-11.
25. The method of 23, wherein the interleukin peptide binding domain comprises amino acids 123-265 of SEQ ID NO: 67, amino acids 21-153 of SEQ ID NO: 68, amino acids 57-210 of SEQ ID NO: 69, amino acids 21-99 or amino acids 31-94 of SEQ ID NO: 70, amino acids 37-173 or amino acids 19-178 of SEQ ID NO: 71, or amino acids 37-199 of SEQ ID NO: 72.
26. The method of 23, wherein the VEGF peptide binding domain is a VEGF-A, a VEGF-B, a VEGF-C, a VEGF-D, or a placenta growth factor (PIGF).
27. The method of 23, wherein the VEGF peptide binding domain comprises amino acids 50-133 of SEQ ID NO: 73, amino acids 45-127 of SEQ ID NO: 74, amino acids 129-214 of SEQ ID NO: 75, amino acids 109-194 of SEQ ID NO: 76, amino acids 46-163, amino acids 49-162, amino acids 168-345, amino acids 244-306, or amino acids 248-340 of SEQ ID NO: 77, or amino acids 50-131 or amino acids 132-203 of SEQ ID NO: 78.
28. The method of 23, wherein the IGF peptide binding domain is an IGF-1 or an IGF-2.
29. The method of 23, wherein the IGF peptide binding domain comprises amino acids 52-109 or amino acids 49-118 of SEQ ID NO: 79, or amino acids 31-84 or amino acids 25-180 of SEQ ID NO: 80.
30. The method of 23, wherein the EGF peptide binding domain an EGF, a heparin-binding EGF-like growth factor (HB-EGF), a transforming growth factor-α (TGF-α), an amphiregulin (AR), an epiregulin (EPR), an epigen (EPG), a betacellulin (BTC), a neuregulin-1 (NRG1), a neuregulin-2 (NRG2), a neuregulin-3, (NRG3), or a neuregulin-4 (NRG4).
31. The method of 30, wherein the NRG-2 is a NRG-2 isoform 1, a NRG-2 isoform 2, a NRG-2 isoform 3, a NRG-2 isoform 4, a NRG-2 isoform 5, or a NRG-2 isoform 6.
32. The method of 23, wherein the EGF peptide binding domain comprises SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91.
33. The method of 23, wherein the EGF peptide binding domain comprises amino acids 101-251 or amino acids 107-251 of SEQ ID NO: 84, amino acids 63-108 of SEQ ID NO: 85, amino acids 23-154 of SEQ ID NO: 86, amino acids 235-630 of SEQ ID NO: 88, amino acids 398-718 of SEQ ID NO: 89, or amino acids 353-648 of SEQ ID NO: 90.
34. The method of 21, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.
35. The method of 21, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.
36. The method of 21, wherein the exogenous protease cleavage site is a plant papain cleavage site, an insect papain cleavage site, a crustacian papain cleavage site, an enterokinase cleavage site, a human rhinovirus 3C protease cleavage site, a human enterovirus 3C protease cleavage site, a tobacco etch virus protease cleavage site, a Tobacco Vein Mottling Virus cleavage site, a subtilisin cleavage site, a hydroxylamine cleavage site, or a Caspase 3 cleavage site.
37. The method of 21, wherein the urogenital-neurological disorder is urinary incontinence, overactive bladder, detrusor dysfunction, lower urinary tract dysfunction, urinary retention, urinary hesitancy, polyuria, nocturia, chronic urinary tract infection, an urogenital disorder associated with a prostate disorder, an urogenital disorder associated with a uterine disorder, or an urogenital disorder associated with a neurogenic dysfunction.
38. The method of 37, wherein the urinary incontinence is an urge urinary incontinence, a stress urinary incontinence, an overflow urinary incontinence, a mixed urinary incontinence, or a continuous urinary incontinence.
39. The method of 37, wherein the detrusor dysfunction is a detrusor overactivity, a detrusor instability, or a detrusor-sphincter dyssynergia.
40. The method of 37, wherein the urogenital disorder associated with a prostate disorder is an urogenital disorder associated with benign prostatic hyperplasia, an urogenital disorder associated with prostatitis, or an urogenital disorder associated with prostatodynia.
41. The method of 37, wherein the urogenital disorder associated with a neurogenic dysfunction is an urogenital disorder associated with Parkinson's Disease, an urogenital disorder associated with multiple sclerosis, an urogenital disorder associated with spina bifida, an urogenital disorder associated with transverse myelitis, an urogenital disorder associated with stroke, an urogenital disorder associated with a spinal cord injury, an urogenital disorder associated with a spasm reflex, an urogenital disorder associated with a neurologic lesion of the spinal cord, or an urogenital disorder associated with a neurologic lesion of the brain.
42. A manufacturing of a medicament for treating urogenital-neurological disorder in a mammal in need thereof, wherein the medicament comprises a TVEMP including a retargeted peptide binding domain, a Clostridial toxin translocation domain and a Clostridial toxin enzymatic domain and wherein administration of a therapeutically effective amount of the medicament to the mammal reduces a symptom of the urogenital-neurological disorder, thereby treating the mammal.
43. A use of a composition for treating urogenital-neurological disorder in a mammal in need thereof, the use comprising the step of administering to the mammal in need thereof a therapeutically effective amount of a composition, wherein the composition comprises a TVEMP including a retargeted peptide binding domain, a Clostridial toxin translocation domain and a Clostridial toxin enzymatic domain and wherein administration of the composition reduces a symptom of the urogenital-neurological disorder, thereby treating the mammal.
44. The medicament of 42 or use of 43, wherein the TVEMP comprises a linear amino-to-carboxyl single polypeptide order of 1) the Clostridial toxin enzymatic domain, the Clostridial toxin translocation domain, the retargeted peptide binding domain, 2) the Clostridial toxin enzymatic domain, the retargeted peptide binding domain, the Clostridial toxin translocation domain, 3) the retargeted peptide binding domain, the Clostridial toxin translocation domain, and the Clostridial toxin enzymatic domain, 4) the retargeted peptide binding domain, the Clostridial toxin enzymatic domain, the Clostridial toxin translocation domain, 5) the Clostridial toxin translocation domain, the Clostridial toxin enzymatic domain and the retargeted peptide binding domain, or 6) the Clostridial toxin translocation domain, the retargeted peptide binding domain and the Clostridial toxin enzymatic domain.
45. The medicament of 42 or use of 43, wherein the retargeted peptide binding domain is an interleukin (IL) peptide binding domain, a vascular endothelial growth factor (VEGF) peptide binding domain, an insulin-like growth factor (IGF) peptide binding domain, or an epidermal growth factor (EGF) peptide binding domain.
46. The medicament or use of 45, wherein the interleukin peptide binding domain is an IL-1, an IL-2, an IL-6, an IL-8, an IL-10, or an IL-11.
47. The medicament or use of 45, wherein the interleukin peptide binding domain comprises amino acids 123-265 of SEQ ID NO: 67, amino acids 21-153 of SEQ ID NO: 68, amino acids 57-210 of SEQ ID NO: 69, amino acids 21-99 or amino acids 31-94 of SEQ ID NO: 70, amino acids 37-173 or amino acids 19-178 of SEQ ID NO: 71, or amino acids 37-199 of SEQ ID NO: 72.
48. The medicament or use of 45, wherein the VEGF peptide binding domain is a VEGF-A, a VEGF-B, a VEGF-C, a VEGF-D, or a placenta growth factor (PIGF).
49. The medicament or use of 45, wherein the VEGF peptide binding domain comprises amino acids 50-133 of SEQ ID NO: 73, amino acids 45-127 of SEQ ID NO: 74, amino acids 129-214 of SEQ ID NO: 75, amino acids 109-194 of SEQ ID NO: 76, amino acids 46-163, amino acids 49-162, amino acids 168-345, amino acids 244-306, or amino acids 248-340 of SEQ ID NO: 77, or amino acids 50-131 or amino acids 132-203 of SEQ ID NO: 78.
50. The medicament or use of 45, wherein the IGF peptide binding domain is an IGF-1 or an IGF-2.
51. The medicament or use of 45, wherein the IGF peptide binding domain comprises amino acids 52-109 or amino acids 49-118 of SEQ ID NO: 79, or amino acids 31-84 or amino acids 25-180 of SEQ ID NO: 80.
52. The medicament or use of 45, wherein the EGF peptide binding domain an EGF, a heparin-binding EGF-like growth factor (HB-EGF), a transforming growth factor-α (TGF-α), an amphiregulin (AR), an epiregulin (EPR), an epigen (EPG), a betacellulin (BTC), a neuregulin-1 (NRG1), a neuregulin-2 (NRG2), a neuregulin-3, (NRG3), or a neuregulin-4 (NRG4).
53. The medicament or use of 52, wherein the NRG-2 is a NRG-2 isoform 1, a NRG-2 isoform 2, a NRG-2 isoform 3, a NRG-2 isoform 4, a NRG-2 isoform 5, or a NRG-2 isoform 6.
54. The medicament or use of 45, wherein the EGF peptide binding domain comprises SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91.
55. The medicament or use of 45, wherein the EGF peptide binding domain comprises amino acids 101-251 or amino acids 107-251 of SEQ ID NO: 84, amino acids 63-108 of SEQ ID NO: 85, amino acids 23-154 of SEQ ID NO: 86, amino acids 235-630 of SEQ ID NO: 88, amino acids 398-718 of SEQ ID NO: 89, or amino acids 353-648 of SEQ ID NO: 90.
56. The medicament of 42 or use of 43, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.
57. The medicament of 42 or use of 43, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.
58. The medicament of 42 or use of 43, wherein the urogenital-neurological disorder is urinary incontinence, overactive bladder, detrusor dysfunction, lower urinary tract dysfunction, urinary retention, urinary hesitancy, polyuria, nocturia, chronic urinary tract infection, an urogenital disorder associated with a prostate disorder, an urogenital disorder associated with a uterine disorder, or an urogenital disorder associated with a neurogenic dysfunction.
59. The medicament or use of 58, wherein the urinary incontinence is an urge urinary incontinence, a stress urinary incontinence, an overflow urinary incontinence, a mixed urinary incontinence, or a continuous urinary incontinence.
60. The medicament or use of 58, wherein the detrusor dysfunction is a detrusor overactivity, a detrusor instability, or a detrusor-sphincter dyssynergia.
61. The medicament or use of 58, wherein the urogenital disorder associated with a prostate disorder is an urogenital disorder associated with benign prostatic hyperplasia, an urogenital disorder associated with prostatitis, or an urogenital disorder associated with prostatodynia.
62. The medicament or use of 58, wherein the urogenital disorder associated with a neurogenic dysfunction is an urogenital disorder associated with Parkinson's Disease, an urogenital disorder associated with multiple sclerosis, an urogenital disorder associated with spina bifida, an urogenital disorder associated with transverse myelitis, an urogenital disorder associated with stroke, an urogenital disorder associated with a spinal cord injury, an urogenital disorder associated with a spasm reflex, an urogenital disorder associated with a neurologic lesion of the spinal cord, or an urogenital disorder associated with a neurologic lesion of the brain.
63. A manufacturing of a medicament for treating urogenital-neurological disorder in a mammal in need thereof, wherein the medicament comprises a TVEMP including a retargeted peptide binding domain, a Clostridial toxin translocation domain and a Clostridial toxin enzymatic domain, and an exogenous protease cleavage site and wherein administration of a therapeutically effective amount of the medicament to the mammal reduces a symptom of the urogenital-neurological disorder, thereby treating the mammal.
64. A use of a composition for treating urogenital-neurological disorder in a mammal in need thereof, the use comprising the step of administering to the mammal in need thereof a therapeutically effective amount of a composition, wherein the composition comprises a TVEMP including a retargeted peptide binding domain, a Clostridial toxin translocation domain, a Clostridial toxin enzymatic domain, and an exogenous protease cleavage site, and wherein administration of the composition reduces a symptom of the urogenital-neurological disorder, thereby treating the mammal.
65. The medicament of 63 or use of 64, wherein the TVEMP comprises a linear amino-to-carboxyl single polypeptide order of 1) the Clostridial toxin enzymatic domain, the exogenous protease cleavage site, the Clostridial toxin translocation domain, the retargeted peptide binding domain, 2) the Clostridial toxin enzymatic domain, the exogenous protease cleavage site, the retargeted peptide binding domain, the Clostridial toxin translocation domain, 3) the retargeted peptide binding domain, the Clostridial toxin translocation domain, the exogenous protease cleavage site and the Clostridial toxin enzymatic domain, 4) the retargeted peptide binding domain, the Clostridial toxin enzymatic domain, the exogenous protease cleavage site, the Clostridial toxin translocation domain, 5) the Clostridial toxin translocation domain, the exogenous protease cleavage site, the Clostridial toxin enzymatic domain and the retargeted peptide binding domain, or 6) the Clostridial toxin translocation domain, the exogenous protease cleavage site, the retargeted peptide binding domain and the Clostridial toxin enzymatic domain.
66. The medicament of 63 or use of 64, wherein the retargeted peptide binding domain is an interleukin (IL) peptide binding domain, a vascular endothelial growth factor (VEGF) peptide binding domain, an insulin-like growth factor (IGF) peptide binding domain, or an epidermal growth factor (EGF) peptide binding domain.
67. The medicament or use of 66, wherein the interleukin peptide binding domain is an IL-1, an IL-2, an IL-6, an IL-8, an IL-10, or an IL-11.
68. The medicament or use of 66, wherein the interleukin peptide binding domain comprises amino acids 123-265 of SEQ ID NO: 67, amino acids 21-153 of SEQ ID NO: 68, amino acids 57-210 of SEQ ID NO: 69, amino acids 21-99 or amino acids 31-94 of SEQ ID NO: 70, amino acids 37-173 or amino acids 19-178 of SEQ ID NO: 71, or amino acids 37-199 of SEQ ID NO: 72.
69. The medicament or use of 66, wherein the VEGF peptide binding domain is a VEGF-A, a VEGF-B, a VEGF-C, a VEGF-D, or a placenta growth factor (PIGF).
70. The medicament or use of 66, wherein the VEGF peptide binding domain comprises amino acids 50-133 of SEQ ID NO: 73, amino acids 45-127 of SEQ ID NO: 74, amino acids 129-214 of SEQ ID NO: 75, amino acids 109-194 of SEQ ID NO: 76, amino acids 46-163, amino acids 49-162, amino acids 168-345, amino acids 244-306, or amino acids 248-340 of SEQ ID NO: 77, or amino acids 50-131 or amino acids 132-203 of SEQ ID NO: 78.
71. The medicament or use of 66, wherein the IGF peptide binding domain is an IGF-1 or an IGF-2.
72. The medicament or use of 66, wherein the IGF peptide binding domain comprises amino acids 52-109 or amino acids 49-118 of SEQ ID NO: 79, or amino acids 31-84 or amino acids 25-180 of SEQ ID NO: 80.
73. The medicament or use of 66, wherein the EGF peptide binding domain an EGF, a heparin-binding EGF-like growth factor (HB-EGF), a transforming growth factor-α (TGF-α), an amphiregulin (AR), an epiregulin (EPR), an epigen (EPG), a betacellulin (BTC), a neuregulin-1 (NRG1), a neuregulin-2 (NRG2), a neuregulin-3, (NRG3), or a neuregulin-4 (NRG4).
74. The medicament or use of 73, wherein the NRG-2 is a NRG-2 isoform 1, a NRG-2 isoform 2, a NRG-2 isoform 3, a NRG-2 isoform 4, a NRG-2 isoform 5, or a NRG-2 isoform 6.
75. The medicament or use of 66, wherein the EGF peptide binding domain comprises SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91.
76. The medicament or use of 66, wherein the EGF peptide binding domain comprises amino acids 101-251 or amino acids 107-251 of SEQ ID NO: 84, amino acids 63-108 of SEQ ID NO: 85, amino acids 23-154 of SEQ ID NO: 86, amino acids 235-630 of SEQ ID NO: 88, amino acids 398-718 of SEQ ID NO: 89, or amino acids 353-648 of SEQ ID NO: 90.
77. The medicament of 63 or use of 64, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.
78. The medicament of 63 or use of 64, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.
79. The medicament of 63 or use of 64, wherein the urogenital-neurological disorder is urinary incontinence, overactive bladder, detrusor dysfunction, lower urinary tract dysfunction, urinary retention, urinary hesitancy, polyuria, nocturia, chronic urinary tract infection, an urogenital disorder associated with a prostate disorder, an urogenital disorder associated with a uterine disorder, or an urogenital disorder associated with a neurogenic dysfunction.
80. The medicament or use of 79, wherein the urinary incontinence is an urge urinary incontinence, a stress urinary incontinence, an overflow urinary incontinence, a mixed urinary incontinence, or a continuous urinary incontinence.
81. The medicament or use of 79, wherein the detrusor dysfunction is a detrusor overactivity, a detrusor instability, or a detrusor-sphincter dyssynergia.
82. The medicament or use of 79, wherein the urogenital disorder associated with a prostate disorder is an urogenital disorder associated with benign prostatic hyperplasia, an urogenital disorder associated with prostatitis, or an urogenital disorder associated with prostatodynia.
83. The medicament or use of 79, wherein the urogenital disorder associated with a neurogenic dysfunction is an urogenital disorder associated with Parkinson's Disease, an urogenital disorder associated with multiple sclerosis, an urogenital disorder associated with spina bifida, an urogenital disorder associated with transverse myelitis, an urogenital disorder associated with stroke, an urogenital disorder associated with a spinal cord injury, an urogenital disorder associated with a spasm reflex, an urogenital disorder associated with a neurologic lesion of the spinal cord, or an urogenital disorder associated with a neurologic lesion of the brain.
84. The medicament of 63 or use of 64, wherein the exogenous protease cleavage site is a plant papain cleavage site, an insect papain cleavage site, a crustacian papain cleavage site, an enterokinase cleavage site, a human rhinovirus 3C protease cleavage site, a human enterovirus 3C protease cleavage site, a tobacco etch virus protease cleavage site, a Tobacco Vein Mottling Virus cleavage site, a subtilisin cleavage site, a hydroxylamine cleavage site, or a Caspase 3 cleavage site.

### EXAMPLES

The following non-limiting examples are provided for illustrative purposes only in order to facilitate a more complete understanding of disclosed embodiments and are in no way intended to limit any of the embodiments disclosed in the present specification.

### Example 1

### Treatment of Urinary Incontinence

A 69 year old female complains of the inability to control the passage of urine. A physician diagnosis the patient with urinary incontinence having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the woman indicates there is improvement of her ability to control the passage of urine. At one and three month check-ups, the woman indicates that she continues to have increased control over her ability to pass urine. This reduction in an urinary incontinence symptom indicates successful treatment with the composition comprising a TVEMP.

A 72 year old female complains of the inability to control the passage of urine, and leakage occurs especially when she coughs, sneezes, laughs or exercises. A physician diagnosis the patient with stress urinary incontinence having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the woman indicates there is improvement of her ability to control the passage of urine, especially when she coughs, sneezes, laughs or exercises. At one and three month check-ups, the woman indicates that she continues to have increased control over her ability to pass urine. This reduction in a stress urinary incontinence symptom indicates successful treatment with the composition comprising a TVEMP.

A 62 year old male complains of the inability to control the passage of urine, experiencing a sudden need to urinate. A physician diagnosis the patient with urge urinary incontinence having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, lower pelvic muscles, prostate, bulbourethral gland, bulb, crus or penis. The patient's condition is monitored and after about 1-3 days from treatment, and the man indicates there is improvement of his ability to control the passage of urine because of a reduced sudden need to urinate. At one and three month check-ups, the man indicates that he continues to have increased control over his ability to pass urine. This reduction in an urge urinary incontinence symptom indicates successful treatment with the composition comprising a TVEMP.

A 58 year old male complains of the inability to control the passage of urine because of leakage that occurs. A physician diagnosis the patient with overflow urinary incontinence having a neurological component involving abnormal neuron activity that is causing blockage. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, lower pelvic muscles, prostate, bulbourethral gland, bulb, crus or penis. The patient's condition is monitored and after about 1-3 days from treatment, and the man indicates there is improvement of his ability to control the passage of urine because of reduced leakage. At one and three month check-ups, the man indicates that he continues to have increased control over his ability to pass urine. This reduction in an overflow urinary incontinence symptom indicates successful treatment with the composition comprising a TVEMP.

### Example 2

### Treatment of Overactive Bladder

A 58 year old male complains of increased urinary urgency. A physician diagnosis the patient with overactive bladder having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, lower pelvic muscles, prostate, bulbourethral gland, bulb, crus or penis. The patient's condition is monitored and after about 1-3 days from treatment, and the man indicates that he has a reduced urgency to urinate. At one and three month check-ups, the man indicates that he continues to have a reduced urgency to urinate. This reduction in an overactive bladder symptom indicates successful treatment with the composition comprising a TVEMP.

A 66 year old female complains of having to wake up several times during the night to urinate. A physician determines that this is nocturia and diagnosis the patient with overactive bladder having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the woman indicates that she has a reduced need to wake up several times during the night to urinate. At one and three month check-ups, the woman indicates that she continues to have a reduced need to wake up several times during the night to urinate. This reduction in an overactive bladder symptom indicates successful treatment with the composition comprising a TVEMP.

A 47 year old female complains of having to urinate several times a day. A physician determines that this is polyuria and diagnosis the patient with overactive bladder having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the woman indicates that she has a reduced need to urinate during the day. At one and three month check-ups, the woman indicates that she continues to have a reduced need urinate during the day. This reduction in an overactive bladder symptom indicates successful treatment with the composition comprising a TVEMP.

A 67 year old male complains of the inability to control the passage of urine because of a sudden need to urinate. A physician determines that this is urge incontinence and diagnosis the patient with overactive bladder having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, lower pelvic muscles, prostate, bulbourethral gland, bulb, crus or penis. The patient's condition is monitored and after about 1-3 days from treatment, and the man indicates that he has a reduced urgency to urinate. At one and three month check-ups, the man indicates that he continues to have a reduced urgency to urinate. This reduction in an overactive bladder symptom indicates successful treatment with the composition comprising a TVEMP.

### Example 3

### Treatment of Detrusor Dysfunction

A 44 year old female complains of uncontrollable bladder contractions. A physician determines that this is uninhibitable bladder contractions and diagnosis the patient with a detrusor dysfunction having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the woman indicates that there is a reduction in uncontrollable bladder contractions. At one and three month check-ups, the woman indicates that she continues to have a reduction in uncontrollable bladder contractions. This reduction in a detrusor dysfunction symptom indicates successful treatment with the composition comprising a TVEMP.

In an alternative scenario, the physician determines that this is uninhibitable bladder contractions and diagnosis the patient with detrusor overactivity having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the woman indicates that there is a reduction in uncontrollable bladder contractions. At one and three month check-ups, the woman indicates that she continues to have a reduction in uncontrollable bladder contractions. This reduction in a detrusor overactivity symptom indicates successful treatment with the composition comprising a TVEMP.

In another alternative scenario, the physician determines that this is uninhibitable bladder contractions and diagnosis the patient with detrusor instability having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the woman indicates that there is a reduction in uncontrollable bladder contractions. At one and three month check-ups, the woman indicates that she continues to have a reduction in uncontrollable bladder contractions. This reduction in a detrusor instability symptom indicates successful treatment with the composition comprising a TVEMP.

A 50 year old female complains of an urgency to urinate. A physician determines that this is urinary urgency and diagnosis the patient with a detrusor dysfunction having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the woman indicates that there is a reduction in the urgency to urinate. At one and three month check-ups, the woman indicates that she continues to have a reduction in the urgency to urinate. This reduction in a detrusor dysfunction symptom indicates successful treatment with the composition comprising a TVEMP.

In an alternative scenario, the physician determines that this is urinary urgency and diagnosis the patient with detrusor overactivity having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the woman indicates that there is a reduction in the urgency to urinate. At one and three month check-ups, the woman indicates that she continues to have a reduction in the urgency to urinate. This reduction in a detrusor overactivity symptom indicates successful treatment with the composition comprising a TVEMP.

In another alternative scenario, the physician determines that this is urinary urgency and diagnosis the patient with detrusor instability having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the woman indicates that there is a reduction in the urgency to urinate. At one and three month check-ups, the woman indicates that she continues to have a reduction in the urgency to urinate. This reduction in a detrusor instability symptom indicates successful treatment with the composition comprising a TVEMP.

A 59 year old male complains of having to urinate all the time. A physician determines that this is urinary frequency and diagnosis the patient with a detrusor dysfunction having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, lower pelvic muscles, prostate, bulbourethral gland, bulb, crus or penis. The patient's condition is monitored and after about 1-3 days from treatment, and the man indicates that there is a reduction in the need to urinate all the time. At one and three month check-ups, the man indicates that he continues to have a reduction in the need to urinate all the time. This reduction in a detrusor dysfunction symptom indicates successful treatment with the composition comprising a TVEMP.

In an alternative scenario, the physician determines that this is urinary frequency and diagnosis the patient with detrusor overactivity having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the man indicates that there is a reduction in the need to urinate all the time. At one and three month check-ups, the man indicates that he continues to have a reduction in the need to urinate all the time. This reduction in a detrusor overactivity symptom indicates successful treatment with the composition comprising a TVEMP.

In another alternative scenario, the physician determines that this is urinary frequency and diagnosis the patient with detrusor instability having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the man indicates that there is a reduction in the need to urinate all the time. At one and three month check-ups, the man indicates that he continues to have a reduction in the need to urinate all the time. This reduction in a detrusor instability symptom indicates successful treatment with the composition comprising a TVEMP.

A 74 year old male complains of the involuntary loss of urine. A physician determines that this is enuresis and diagnosis the patient with a detrusor dysfunction having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, lower pelvic muscles, prostate, bulbourethral gland, bulb, crus or penis. The patient's condition is monitored and after about 1-3 days from treatment, and the man indicates that there is a reduction in the involuntary loss of urine. At one and three month check-ups, the man indicates that he continues to have a reduction in the involuntary loss of urine. This reduction in a detrusor dysfunction symptom indicates successful treatment with the composition comprising a TVEMP.

In an alternative scenario, the physician determines that this is enuresis and diagnosis the patient with detrusor overactivity having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the man indicates that there is a reduction in the involuntary loss of urine. At one and three month check-ups, the man indicates that he continues to have a reduction in the involuntary loss of urine. This reduction in a detrusor overactivity symptom indicates successful treatment with the composition comprising a TVEMP.

In another alternative scenario, the physician determines that this is enuresis and diagnosis the patient with detrusor instability having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the man indicates that there is a reduction in the involuntary loss of urine. At one and three month check-ups, the man indicates that he continues to have a reduction in the involuntary loss of urine. This reduction in a detrusor instability symptom indicates successful treatment with the composition comprising a TVEMP.

A 63 year old male complains of having to wake up several times during the night to urinate. A physician determines that this is nocturia and diagnosis the patient with a detrusor dysfunction having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, lower pelvic muscles, prostate, bulbourethral gland, bulb, crus or penis. The patient's condition is monitored and after about 1-3 days from treatment, and the man indicates that there is a reduction in need to wake up several times during the night to urinate. At one and three month check-ups, the man indicates that he continues to have a reduction in need to wake up several times during the night to urinate. This reduction in a detrusor dysfunction symptom indicates successful treatment with the composition comprising a TVEMP.

In an alternative scenario, the physician determines that this is nocturia and diagnosis the patient with detrusor overactivity having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the man indicates that there is a reduction in need to wake up several times during the night to urinate. At one and three month check-ups, the man indicates that he continues to have a reduction in need to wake up several times during the night to urinate. This reduction in a detrusor overactivity symptom indicates successful treatment with the composition comprising a TVEMP.

In another alternative scenario, the physician determines that this is nocturia and diagnosis the patient with detrusor instability having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the man indicates that there is a reduction in need to wake up several times during the night to urinate. At one and three month check-ups, the man indicates that he continues to have a reduction in need to wake up several times during the night to urinate. This reduction in a detrusor instability symptom indicates successful treatment with the composition comprising a TVEMP.

A 61 year old female complains of having to urinate several times a day. A physician determines that this is polyuria and diagnosis the patient with a detrusor dysfunction having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the woman indicates that there is a reduction in the need to urinate several times a day. At one and three month check-ups, the woman indicates that she continues to have a reduction in the need to urinate several times a day. This reduction in a detrusor dysfunction symptom indicates successful treatment with the composition comprising a TVEMP.

In an alternative scenario, the physician determines that this is polyuria and diagnosis the patient with detrusor overactivity having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the woman indicates that there is a reduction in the need to urinate several times a day. At one and three month check-ups, the woman indicates that she continues to have a reduction in the need to urinate several times a day. This reduction in a detrusor overactivity symptom indicates successful treatment with the composition comprising a TVEMP.

In another alternative scenario, the physician determines that this is polyuria and diagnosis the patient with detrusor instability having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the woman indicates that there is a reduction in the need to urinate several times a day. At one and three month check-ups, the woman indicates that she continues to have a reduction in the need to urinate several times a day. This reduction in a detrusor instability symptom indicates successful treatment with the composition comprising a TVEMP.

A 65 year old female complains of the inability to control the passage of urine. A physician determines that this is urinary incontinence and diagnosis the patient with a detrusor dysfunction having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from the treatment, and the woman indicates there is improvement of her ability to control the passage of urine. At one and three month check-ups, the woman indicates that she continues to have an improved ability to control the passage of urine since the treatment. This reduction in a detrusor dysfunction symptom indicates successful treatment with the composition comprising a TVEMP.

In an alternative scenario, the physician determines that this is urinary incontinence and diagnosis the patient with detrusor overactivity having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from the treatment, and the woman indicates there is improvement of her ability to control the passage of urine. At one and three month check-ups, the woman indicates that she continues to have an improved ability to control the passage of urine since the treatment. This reduction in a detrusor overactivity symptom indicates successful treatment with the composition comprising a TVEMP.

In another alternative scenario, the physician determines that this is urinary incontinence and diagnosis the patient with detrusor instability having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from the treatment, and the woman indicates there is improvement of her ability to control the passage of urine. At one and three month check-ups, the woman indicates that she continues to have an improved ability to control the passage of urine since the treatment. This reduction in a detrusor instability symptom indicates successful treatment with the composition comprising a TVEMP.

A 55 year old female complains of an interruption of urine flow when she urinates. A physician diagnosis the patient with a detrusor dysfunction having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the woman indicates that there is a reduction in urine flow interruption. At one and three month check-ups, the woman indicates that she continues to have a reduced urine flow interruption since the treatment. This reduction in a detrusor dysfunction symptom indicates successful treatment with the composition comprising a TVEMP.

In an alternative scenario, the physician diagnosis the patient with a detrusor-sphincter dyssynergia having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the woman indicates that there is a reduction in urine flow interruption. At one and three month check-ups, the woman indicates that she continues to have a reduced urine flow interruption since the treatment. This reduction in a detrusor-sphincter dyssynergia symptom indicates successful treatment with the composition comprising a TVEMP.

A 53 year old male complains of increased bladder pressure. A physician determines that this is raised detrusor pressure and diagnosis the patient with a detrusor dysfunction having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, lower pelvic muscles, prostate, bulbourethral gland, bulb, crus or penis. The patient's condition is monitored and after about 1-3 days from treatment, and the man indicates that there is a reduction in bladder pressure. At one and three month check-ups, the man indicates that he continues to have a reduced bladder pressure since the treatment. This reduction in a detrusor dysfunction symptom indicates successful treatment with the composition comprising a TVEMP.

In an alternative scenario, the physician determines that this is raised detrusor pressure and diagnosis the patient with a detrusor-sphincter dyssynergia having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the man indicates that there is a reduction in bladder pressure. At one and three month check-ups, the man indicates that he continues to have a reduced bladder pressure since the treatment. This reduction in a detrusor-sphincter dyssynergia symptom indicates successful treatment with the composition comprising a TVEMP.

A 75 year old male complains of the inability to urinate. A physician determines that this is urinary retention and diagnosis the patient with a detrusor dysfunction having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, lower pelvic muscles, prostate, bulbourethral gland, bulb, crus or penis. The patient's condition is monitored and after about 1-3 days from treatment, and the man indicates that he has regained the ability to urinate. At one and three month check-ups, the man indicates that he continues to have the ability to urinate. This reduction in a detrusor dysfunction symptom indicates successful treatment with the composition comprising a TVEMP.

In an alternative scenario, the physician determines that this is urinary retention and diagnosis the patient with a detrusor-sphincter dyssynergia having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the man indicates that he has regained the ability to urinate. At one and three month check-ups, the man indicates that he continues to have the ability to urinate. This reduction in a detrusor-sphincter dyssynergia symptom indicates successful treatment with the composition comprising a TVEMP.

### Example 4

### Treatment of Lower Urinary Tract Dysfunction

A 69 year old male complains of the need to urinate suddenly. A physician determines that this is a urine storage problem and diagnosis the patient with a lower urinary tract dysfunction having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, lower pelvic muscles, prostate, bulbourethral gland, bulb, crus or penis. The patient's condition is monitored and after about 1-3 days from treatment, and the man indicates that there is a reduction in the sudden need to urinate. At one and three month check-ups, the man indicates that he still experiences a reduced need to urinate. This reduction in a lower urinary tract dysfunction indicates successful treatment with the composition comprising a TVEMP. In similar scenarios the patient could have complained of other storage symptoms of lower urinary tract dysfunction such as, e.g., urinary frequency, enuresis, polyuria, nocturia increased bladder sensation, decreased bladder sensation, absent bladder sensation, non-specific bladder sensation, and/or urinary incontinence. In each case, after diagnosis of lower urinary tract dysfunction, a physician would treat the patient as indicated above and there would be a reduction in the lower urinary tract dysfunction storage symptom.

A 70 year old male complains of having difficulty urinating and having to strain in order to urinate. A physician determines that this is a urine voiding problem and diagnosis the patient with a lower urinary tract dysfunction having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, lower pelvic muscles, prostate, bulbourethral gland, bulb, crus or penis. The patient's condition is monitored and after about 1-3 days from treatment, and the man indicates that it is easier to urinate and he does not have to strain as much in order to urinate. At one and three month check-ups, the man indicates that he still experiences an easier time to urinate. This reduction in a lower urinary tract dysfunction indicates successful treatment with the composition comprising a TVEMP. In similar scenarios the patient could have complained of other voiding symptoms of lower urinary tract dysfunction such as, e.g., reduced urine flow, splitting or spraying of urine, intermittent urine flow, urinary hesitancy, and/or terminal dribble of urine. In each case, after diagnosis of lower urinary tract dysfunction, a physician would treat the patient as indicated above and there would be a reduction in the lower urinary tract dysfunction voiding symptom.

A 77 year old male complains of urine dribbling after he finishes urinating. A physician determines that this is a urine post-micturition problem and diagnosis the patient with a lower urinary tract dysfunction having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, lower pelvic muscles, prostate, bulbourethral gland, bulb, crus or penis. The patient's condition is monitored and after about 1-3 days from treatment, and the man indicates that there is a reduction in urine dribbling after he finishes urinating. At one and three month check-ups, the man indicates that he still experiences reduced dribbling after he finishes urinating. This reduction in a lower urinary tract dysfunction indicates successful treatment with the composition comprising a TVEMP. In similar scenarios the patient could have complained of other post-micturition symptoms of lower urinary tract dysfunction such as, e.g., sensation of incomplete emptying. In each case, after diagnosis of lower urinary tract dysfunction, a physician would treat the patient as indicated above and there would be a reduction in the lower urinary tract dysfunction post-micturition symptom.

### Example 5

### Treatment of Urinary Retention

A 79 year old female complains that she cannot urinate. A physician diagnosis the patient with urinary retention having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the woman indicates that she has regained the ability to urinate. At one and three month check-ups, the woman indicates that she still continues to have control over her ability to urinate. This reduction in a urinary retention symptom indicates successful treatment with the composition comprising a TVEMP.

### Example 6

### Treatment of Urinary Hesitancy

A 78 year old male complains that he has difficulty starting and/or maintaining his ability to urinate. A physician diagnosis the patient with urinary hesitancy having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, lower pelvic muscles, prostate, bulbourethral gland, bulb, crus or penis. The patient's condition is monitored and after about 1-3 days from treatment, and the man indicates that he has less difficulty in starting and/or maintaining his ability to urinate. At one and three month check-ups, the man indicates that he still experiences less difficulty in starting and/or maintaining his ability to urinate. This reduction in a urinary hesitancy symptom indicates successful treatment with the composition comprising a TVEMP.

### Example 7

### Treatment of Polyuria

A 68 year old male complains that he has to urinate all the time during the day. A physician diagnosis the patient with polyuria having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, lower pelvic muscles, prostate, bulbourethral gland, bulb, crus or penis. The patient's condition is monitored and after about 1-3 days from treatment, and the man indicates that does not have to urinate as many times during the day as before the treatment. At one and three month check-ups, the man still indicates that does not have to urinate as many times during the day as before the treatment. This reduction in a polyuria symptom indicates successful treatment with the composition comprising a TVEMP.

### Example 8

### Treatment of Nocturia

A 57 year old female complains that she has to wake up several times during the night in order to urinate. A physician diagnosis the patient with nocturia having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the woman indicates that she does not have to get up as many times during the night to urinate as she did before the treatment. At one and three month check-ups, the woman still indicates that she does not have to get up as many times during the night to urinate as she did before the treatment. This reduction in a nocturia symptom indicates successful treatment with the composition comprising a TVEMP.

### Example 9

### Treatment of Chronic Urinary Tract Infection

A 76 year old female complains that she has urinary tract infections all the time. A physician determines that the chrionic urinary tract infections is abacterial and diagnosis the patient with urogential disorder having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the physician indicates that she does not have a urinary tract infection. At one and three month check-ups, the woman indicates that she has not had a urinary tract infection since the treatment. This reduction in a urinary tract infection symptom indicates successful treatment with the composition comprising a TVEMP.

A 75 year old female complains that she has urinary tract infections all the time. A physician determines that the chrionic urinary tract infection is due to vesicoureteral reflux and diagnosis the patient with urogential disorder having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the physician determines that the abnormal backup of urine from the bladder to the kidneys is reduced in the patient. At one and three month check-ups, the woman indicates that she has not had a urinary tract infection since the treatment. This reduction in a urinary tract infection symptom indicates successful treatment with the composition comprising a TVEMP.

### Example 10

### Treatment of Urogenital Disorder Associated with a Prostate Disorder

A 78 year old male complains that he has difficulty starting and/or maintaining his ability to urinate. A physician determines that he has benign prostatic hyperplasia and that this enlargement is blocking the normal flow of urine. The physician diagnosis the patient with urinary hesitancy associated with benign prostatic hyperplasia having a neurological component involving abnormal neuron activity. The man is treated by injecting a composition comprising a TVEMP as disclosed in the present specification into the prostate and/or in the surrounding area of the prostate depending on the location of abnormal neuron activity. The patient's condition is monitored and after about 1-2 weeks from the treatment, the man indicates that he has less difficulty in starting and/or maintaining his ability to urinate. The physician determines that the size of the prostate has reduced since the treatment. At one and three month check-ups, the man indicates that he still experiences less difficulty in starting and/or maintaining his ability to urinate. This reduction in a urinary hesitancy symptom associated with benign prostatic hyperplasia indicates successful treatment with the composition comprising a TVEMP.

### Example 11

### Treatment of Urogenital Disorder Associated with a Neurogenic Dysfunction

A 81 year old female diagnosed with Parkinson's Disease complains about having a sudden need to urinate. A physician determines that this urinary urgency is due to her Parkinson's Disease and diagnosis the patient with urogential disorder associated with a neurogenic dysfunction having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the woman indicates that there is a reduction in the sudden need to urinate. At one and three month check-ups, the woman indicates that she continues to experience a reduced sudden need to urinate. This reduction in a urogenital disorder symptom associated with a neurogenic dysfunction indicates successful treatment with the composition comprising a TVEMP.

A 39 year old female diagnosed with multiple sclerosis complains about having a need to urinate all the time. A physician determines that this urinary frequency is due to her multiple sclerosis and diagnosis the patient with urogential disorder associated with a neurogenic dysfunction having a neurological component involving abnormal neuron activity. The woman is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the woman indicates that there is a reduction in the need to urinate all the time. At one and three month check-ups, the woman indicates that she still experiences a reduced need to urinate all the time. This reduction in a urogenital disorder symptom associated with a neurogenic dysfunction indicates successful treatment with the composition comprising a TVEMP.

A 12 year old male diagnosed with spina bifida complains about the inability to control the passage of urine. A physician determines that this urinary incontinence is due to his spina bifida and diagnosis the patient with urogential disorder associated with a neurogenic dysfunction having a neurological component involving abnormal neuron activity. The boy is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, lower pelvic muscles, prostate, bulbourethral gland, bulb, crus or penis. The patient's condition is monitored and after about 1-3 days from treatment, and the boy indicates that he has an increased ability to control the passage or urine. At one and three month check-ups, the boy indicates that he still experiences an increased ability to control the passage or urine. This reduction in a urogenital disorder symptom associated with a neurogenic dysfunction indicates successful treatment with the composition comprising a TVEMP.

A 84 year old male who experienced a stroke complains about not being able to urinate. A physician determines that this urinary retention is due to his stroke and diagnosis the patient with urogential disorder associated with a neurogenic dysfunction having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, lower pelvic muscles, prostate, bulbourethral gland, bulb, crus or penis. The patient's condition is monitored and after about 1-3 days from treatment, and the man indicates that he can urinate. At one and three month check-ups, the man indicates that he continues to experience the ability to urinate. This reduction in a urogenital disorder symptom associated with a neurogenic dysfunction indicates successful treatment with the composition comprising a TVEMP.

A 23 year old man suffering from a spinal cord injury resulting from a car accident complains about the inability to control the passage of urine. A physician determines that this urinary incontinence is due to his spinal cord injury and diagnosis the patient with urogential disorder associated with a neurogenic dysfunction having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, or lower pelvic muscles. The patient's condition is monitored and after about 1-3 days from treatment, and the man indicates that he has an increased ability to control the passage or urine. At one and three month check-ups, the man indicates that he still experiences an increased ability to control the passage or urine. This reduction in a urogenital disorder symptom associated with a neurogenic dysfunction indicates successful treatment with the composition comprising a TVEMP.

A 63 year old male who has cancerous lesion in his brain complains about having a need to urinate all the time. A physician determines that this urinary frequency is due to his lesion and diagnosis the patient with urogential disorder associated with a neurogenic dysfunction having a neurological component involving abnormal neuron activity. The man is treated by injecting urethroscopically a composition comprising a TVEMP as disclosed in the present specification. Depending on the location of abnormal neuron activity, the toxin can be administered into e.g., the detrusor, the bladder neck including the external and internal urethral sphincters, the trigone, the bladder dome or other areas of the bladder wall, and/or other areas surrounding the bladder, such as the urethra, ureter, urogenital diaphragm, lower pelvic muscles, prostate, bulbourethral gland, bulb, crus or penis. The patient's condition is monitored and after about 1-3 days from treatment, and the man indicates that there is a reduction in the need to urinate all the time. At one and three month check-ups, the man indicates that he still experiences a reduced need to urinate all the time. This reduction in a urogenital disorder symptom associated with a neurogenic dysfunction indicates successful treatment with the composition comprising a TVEMP.

In closing, it is to be understood that although aspects of the present specification have been described with reference to the various embodiments, one skilled in the art will readily appreciate that the specific examples disclosed are only illustrative of the principles of the subject matter disclosed in the present specification. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing quantities of ingredients, properties such as molecular weight, reaction conditions, and so forth used in the specification and claims are to be understood as being modified in all instances by the term “about.” As used herein, the term “about” when qualifying a value of a stated item, number, percentage, parameter, or term refers to a range of plus or minus ten percent of the value of the stated item, number, percentage, parameter, or term. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

All patents, patent publications, and other publications referenced and identified in the present specification are individually and expressly incorporated herein by reference in their entirety for the purpose of describing and disclosing, e.g., the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed:

1. A method of treating urogenital-neurological disorder in a mammal, the method comprising the step of administering to the mammal in need thereof a therapeutically effective amount of a composition including a TVEMP comprising a retargeted peptide binding domain, a Clostridial toxin translocation domain and a Clostridial toxin enzymatic domain,
wherein the retargeted peptide binding domain is an interleukin (IL) peptide binding domain, a vascular endothelial growth factor (VEGF) peptide binding domain, an insulin-like growth factor (IGF) peptide binding domain, or an epidermal growth factor (EGF) peptide binding domain, and
wherein administration of the composition reduces a symptom of the urogenital-neurological disorder, thereby treating the mammal.
